SEQUENCE LISTING (120 sequences)

SEQ ID NO: 1 - protein, 1255 aa, Artificial Sequence
  full-length MUC1 Receptor
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 2 - protein, 19 aa, Artificial Sequence
  N-terminal MUC-1 signaling sequence
MTPGTQSPFFLLLLLTVLT

SEQ ID NO: 3 - protein, 23 aa, Artificial Sequence
  N-terminal MUC-1 signaling sequence
MTPGTQSPFFLLLLLTVLTVVTA

SEQ ID NO: 4 - protein, 23 aa, Artificial Sequence
  N-terminal MUC-1 signaling sequence
MTPGTQSPFFLLLLLTVLTVVTG

SEQ ID NO: 5 - protein, 146 aa, Artificial Sequence
  truncated MUC1 receptor isoform
GTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 6 - protein, 45 aa, Artificial Sequence
  extracellular domain of Native Primary Sequence
GTINVHDVETQFNQYKTEAASRYNLTISDVSVSDVPFPFSAQSGA

SEQ ID NO: 7 - protein, 44 aa, Artificial Sequence
  extracellular domain of Native Primary Sequence
TINVHDVETQFNQYKTEAASRYNLTISDVSVSDVPFPFSAQSGA

SEQ ID NO: 8 - protein, 45 aa, Artificial Sequence
  extracellular domain of “SPY” functional variant
GTINVHDVETQFNQYKTEAASPYNLTISDVSVSDVPFPFSAQSGA

SEQ ID NO: 9 - protein, 44 aa, Artificial Sequence
  extracellular domain of “SPY” functional variant
TINVHDVETQFNQYKTEAASPYNLTISDVSVSDVPFPFSAQSGA

SEQ ID NO: 10 - DNA, 216 bp, Artificial Sequence
  MUC1 cytoplasmic domain nucleotide sequence
tgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaacggtggcagcagcctctcttacacaaacccagcagtggcagccgcttctgccaacttg

SEQ ID NO: 11 - protein, 72 aa, Artificial Sequence
  MUC1 cytoplasmic domain amino acid sequence
CQCRRKNYGQLDIFPARDTYHPMSEYPTYHTHGRYVPPSSTDRSPYEKVSAGNGGSSLSYTNPAVAAASANL

SEQ ID NO: 12 - DNA, 854 bp, Artificial Sequence
  NME7 nucleotide sequence
gagatcctgagacaatgaatcatagtgaaagattcgttttcattgcagagtggtatgatccaaatgcttcacttcttcgacgttatgagcttttattttacccaggggatggatctgttgaaatgcatgatgtaaagaatcatcgcacctttttaaagcggaccaaatatgataacctgcacttggaagatttatttataggcaacaaagtgaatgtcttttctcgacaactggtattaattgactatggggatcaatatacagctcgccagctgggcagtaggaaagaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggtatgttgaatacactatattcagtacattttgttaataggagagcaatgtttattttcttgatgtactttatgtatagaaaataa

SEQ ID NO: 13 - protein, 283 aa, Artificial Sequence
  NME7 amino acid sequence
DPETMNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGMLNTLYSVHFVNRRAMFIFLMYFMYRK

SEQ ID NO: 14 - DNA, 534 bp, Artificial Sequence
  NM23-H1 nucleotide sequence
atggtgctactgtctactttagggatcgtctttcaaggcgaggggcctcctatctcaagctgtgatacaggaaccatggccaactgtgagcgtaccttcattgcgatcaaaccagatggggtccagcggggtcttgtgggagagattatcaagcgttttgagcagaaaggattccgccttgttggtctgaaattcatgcaagcttccgaagatcttctcaaggaacactacgttgacctgaaggaccgtccattctttgccggcctggtgaaatacatgcactcagggccggtagttgccatggtctgggaggggctgaatgtggtgaagacgggccgagtcatgctcggggagaccaaccctgcagactccaagcctgggaccatccgtggagacttctgcatacaagttggcaggaacattatacatggcagtgattctgtggagagtgcagagaaggagatcggcttgtggtttcaccctgaggaactggtagattacacgagctgtgctcagaactggatctatgaatga

SEQ ID NO: 15 - protein, 177 aa, Artificial Sequence
  NM23-H1 describes amino acid sequence
MVLLSTLGIVFQGEGPPISSCDTGTMANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRNIIHGSDSVESAEKEIGLWFHPEELVDYTSCAQNWIYE

SEQ ID NO: 16 - DNA, 534 bp, Artificial Sequence
  NM23-H1 S120G mutant nucleotide sequence
atggtgctactgtctactttagggatcgtctttcaaggcgaggggcctcctatctcaagctgtgatacaggaaccatggccaactgtgagcgtaccttcattgcgatcaaaccagatggggtccagcggggtcttgtgggagagattatcaagcgttttgagcagaaaggattccgccttgttggtctgaaattcatgcaagcttccgaagatcttctcaaggaacactacgttgacctgaaggaccgtccattctttgccggcctggtgaaatacatgcactcagggccggtagttgccatggtctgggaggggctgaatgtggtgaagacgggccgagtcatgctcggggagaccaaccctgcagactccaagcctgggaccatccgtggagacttctgcatacaagttggcaggaacattatacatggcggtgattctgtggagagtgcagagaaggagatcggcttgtggtttcaccctgaggaactggtagattacacgagctgtgctcagaactggatctatgaatga

SEQ ID NO: 17 - protein, 177 aa, Artificial Sequence
  NM23-H1 S120G mutant amino acid sequence
MVLLSTLGIVFQGEGPPISSCDTGTMANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRNIIHGGDSVESAEKEIGLWFHPEELVDYTSCAQNWIYE

SEQ ID NO: 18 - DNA, 459 bp, Artificial Sequence
  NM23-H2 nucleotide sequence
atggccaacctggagcgcaccttcatcgccatcaagccggacggcgtgcagcgcggcctggtgggcgagatcatcaagcgcttcgagcagaagggattccgcctcgtggccatgaagttcctccgggcctctgaagaacacctgaagcagcactacattgacctgaaagaccgaccattcttccctgggctggtgaagtacatgaactcagggccggttgtggccatggtctgggaggggctgaacgtggtgaagacaggccgagtgatgcttggggagaccaatccagcagattcaaagccaggcaccattcgtggggacttctgcattcaggttggcaggaacatcattcatggcagtgattcagtaaaaagtgctgaaaaagaaatcagcctatggtttaagcctgaagaactggttgactacaagtcttgtgctcatgactgggtctatgaataa

SEQ ID NO: 19 - protein, 152 aa, Artificial Sequence
  NM23-H2 amino acid sequence
MANLERTFIAIKPDGVQRGLVGEIIKRFEQKGFRLVAMKFLRASEEHLKQHYIDLKDRPFFPGLVKYMNSGPVVAMVWEGLNVVKTGRVMLGETNPADSKPGTIRGDFCIQVGRNIIHGSDSVKSAEKEISLWFKPEELVDYKSCAHDWVYE

SEQ ID NO: 20 - DNA, 1023 bp, Artificial Sequence
  Human NM23-H7-2 sequence optimized for E. coli expression
atgcatgacgttaaaaatcaccgtacctttctgaaacgcacgaaatatgataatctgcatctggaagacctgtttattggcaacaaagtcaatgtgttctctcgtcagctggtgctgatcgattatggcgaccagtacaccgcgcgtcaactgggtagtcgcaaagaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttcaaaattctggataattga

SEQ ID NO: 21 - protein, 340 aa, Artificial Sequence
  Human NM23-H7-2 sequence optimized for E. coli expression
MHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 22 - DNA, 399 bp, Artificial Sequence
  Human NME7-A
atggaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgtttttttga

SEQ ID NO: 23 - protein, 132 aa, Artificial Sequence
  Human NME7-A
MEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFF

SEQ ID NO: 24 - DNA, 444 bp, Artificial Sequence
  Human NME7-A1
atggaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttacttga

SEQ ID NO: 25 - protein, 147 aa, Artificial Sequence
  Human NME7-A1
MEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFT

SEQ ID NO: 26 - DNA, 669 bp, Artificial Sequence
  Human NME7-A2
atgaatcatagtgaaagattcgttttcattgcagagtggtatgatccaaatgcttcacttcttcgacgttatgagcttttattttacccaggggatggatctgttgaaatgcatgatgtaaagaatcatcgcacctttttaaagcggaccaaatatgataacctgcacttggaagatttatttataggcaacaaagtgaatgtcttttctcgacaactggtattaattgactatggggatcaatatacagctcgccagctgggcagtaggaaagaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgtttttttga

SEQ ID NO: 27 - protein, 222 aa, Artificial Sequence
  Human NME7-A2
MNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFF

SEQ ID NO: 28 - DNA, 714 bp, Artificial Sequence
  Human NME7-A3
atgaatcatagtgaaagattcgttttcattgcagagtggtatgatccaaatgcttcacttcttcgacgttatgagcttttattttacccaggggatggatctgttgaaatgcatgatgtaaagaatcatcgcacctttttaaagcggaccaaatatgataacctgcacttggaagatttatttataggcaacaaagtgaatgtcttttctcgacaactggtattaattgactatggggatcaatatacagctcgccagctgggcagtaggaaagaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttacttga

SEQ ID NO: 29 - protein, 237 aa, Artificial Sequence
  Human NME7-A3
MNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFT

SEQ ID NO: 30 - DNA, 408 bp, Artificial Sequence
  Human NME7-B
atgaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttctga

SEQ ID NO: 31 - protein, 135 aa, Artificial Sequence
  Human NME7-B
MNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFF

SEQ ID NO: 32 - DNA, 426 bp, Artificial Sequence
  Human NME7-B1
atgaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattagtga

SEQ ID NO: 33 - protein, 140 aa, Artificial Sequence
  Human NME7-B1
MNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 34 - DNA, 453 bp, Artificial Sequence
  Human NME7-B2
atgccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttctga

SEQ ID NO: 35 - protein, 150 aa, Artificial Sequence
  Human NME7-B2
MPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFF

SEQ ID NO: 36 - DNA, 471 bp, Artificial Sequence
  Human NME7-B3
atgccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattagtga

SEQ ID NO: 37 - protein, 155 aa, Artificial Sequence
  Human NME7-B3
MPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 38 - DNA, 864 bp, Artificial Sequence
  Human NME7-AB
atggaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattagtga

SEQ ID NO: 39 - protein, 286 aa, Artificial Sequence
  Human NME7-AB
MEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 40 - DNA, 846 bp, Artificial Sequence
  Human NME7-AB1
atggaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttctga

SEQ ID NO: 41 - protein, 281 aa, Artificial Sequence
  Human NME7-AB1
MEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFF

SEQ ID NO: 42 - DNA, 399 bp, Artificial Sequence
  Human NME7-A sequence optimized for E. coli expression
atggaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttctga

SEQ ID NO: 43 - protein, 132 aa, Artificial Sequence; identical to SEQ ID NO: 23
  Human NME7-A sequence optimized for E. coli expression

SEQ ID NO: 44 - DNA, 444 bp, Artificial Sequence
  Human NME7-A1 sequence optimized for E. coli expression
atggaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttacctga

SEQ ID NO: 45 - protein, 147 aa, Artificial Sequence; identical to SEQ ID NO: 25
  Human NME7-A1 sequence optimized for E. coli expression

SEQ ID NO: 46 - DNA, 669 bp, Artificial Sequence
  Human NME7-A2 sequence optimized for E. coli expression
atgaatcactccgaacgctttgtttttatcgccgaatggtatgacccgaatgcttccctgctgcgccgctacgaactgctgttttatccgggcgatggtagcgtggaaatgcatgacgttaaaaatcaccgtacctttctgaaacgcacgaaatatgataatctgcatctggaagacctgtttattggcaacaaagtcaatgtgttctctcgtcagctggtgctgatcgattatggcgaccagtacaccgcgcgtcaactgggtagtcgcaaagaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttctga

SEQ ID NO: 47 - protein, 222 aa, Artificial Sequence; identical to SEQ ID NO: 27
  Human NME7-A2 sequence optimized for E. coli expression

SEQ ID NO: 48 - DNA, 714 bp, Artificial Sequence
  Human NME7-A3 sequence optimized for E. coli expression
atgaatcactccgaacgctttgtttttatcgccgaatggtatgacccgaatgcttccctgctgcgccgctacgaactgctgttttatccgggcgatggtagcgtggaaatgcatgacgttaaaaatcaccgtacctttctgaaacgcacgaaatatgataatctgcatctggaagacctgtttattggcaacaaagtcaatgtgttctctcgtcagctggtgctgatcgattatggcgaccagtacaccgcgcgtcaactgggtagtcgcaaagaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttacctga

SEQ ID NO: 49 - protein, 237 aa, Artificial Sequence; identical to SEQ ID NO: 29
  Human NME7-A3 sequence optimized for E. coli expression

SEQ ID NO: 50 - DNA, 408 bp, Artificial Sequence
  Human NME7-B sequence optimized for E. coli expression
atgaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttctga

SEQ ID NO: 51 - protein, 135 aa, Artificial Sequence; identical to SEQ ID NO: 31
  Human NME7-B sequence optimized for E. coli expression

SEQ ID NO: 52 - DNA, 423 bp, Artificial Sequence
  Human NME7-B1 sequence optimized for E. coli expression
atgaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttcaaaattctggataattga

SEQ ID NO: 53 - protein, 140 aa, Artificial Sequence; identical to SEQ ID NO: 33
  Human NME7-B1 sequence optimized for E. coli expression

SEQ ID NO: 54 - DNA, 453 bp, Artificial Sequence
  human NME7-B2 sequence optimized for E. coli expression
atgccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttctga

SEQ ID NO: 55 - protein, 150 aa, Artificial Sequence; identical to SEQ ID NO: 35
  human NME7-B2 sequence optimized for E. coli expression

SEQ ID NO: 56 - DNA, 468 bp, Artificial Sequence
  Human NME7-B3 sequence optimized for E. coli expression
atgccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttcaaaattctggataattga

SEQ ID NO: 57 - protein, 155 aa, Artificial Sequence; identical to SEQ ID NO: 37
  Human NME7-B3 sequence optimized for E. coli expression

SEQ ID NO: 58 - DNA, 861 bp, Artificial Sequence
  Human NME7-AB sequence optimized for E. coli expression
atggaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttcaaaattctggataattga

SEQ ID NO: 59 - protein, 286 aa, Artificial Sequence; identical to SEQ ID NO: 39
  Human NME7-AB sequence optimized for E. coli expression

SEQ ID NO: 60 - DNA, 846 bp, Artificial Sequence
  Human NME7-AB1 sequence optimized for E. coli expression
atggaaaaaacgctggccctgattaaaccggatgcaatctccaaagctggcgaaattatcgaaattatcaacaaagcgggtttcaccatcacgaaactgaaaatgatgatgctgagccgtaaagaagccctggattttcatgtcgaccaccagtctcgcccgtttttcaatgaactgattcaattcatcaccacgggtccgattatcgcaatggaaattctgcgtgatgacgctatctgcgaatggaaacgcctgctgggcccggcaaactcaggtgttgcgcgtaccgatgccagtgaatccattcgcgctctgtttggcaccgatggtatccgtaatgcagcacatggtccggactcattcgcatcggcagctcgtgaaatggaactgtttttcccgagctctggcggttgcggtccggcaaacaccgccaaatttaccaattgtacgtgctgtattgtcaaaccgcacgcagtgtcagaaggcctgctgggtaaaattctgatggcaatccgtgatgctggctttgaaatctcggccatgcagatgttcaacatggaccgcgttaacgtcgaagaattctacgaagtttacaaaggcgtggttaccgaatatcacgatatggttacggaaatgtactccggtccgtgcgtcgcgatggaaattcagcaaaacaatgccaccaaaacgtttcgtgaattctgtggtccggcagatccggaaatcgcacgtcatctgcgtccgggtaccctgcgcgcaatttttggtaaaacgaaaatccagaacgctgtgcactgtaccgatctgccggaagacggtctgctggaagttcaatactttttctga

SEQ ID NO: 61 - protein, 281 aa, Artificial Sequence; identical to SEQ ID NO: 41
  Human NME7-AB1 sequence optimized for E. coli expression

SEQ ID NO: 62 - DNA, 570 bp, Artificial Sequence
  Mouse NME6
atgacctccatcttgcgaagtccccaagctcttcagctcacactagccctgatcaagcctgatgcagttgcccacccactgatcctggaggctgttcatcagcagattctgagcaacaagttcctcattgtacgaacgagggaactgcagtggaagctggaggactgccggaggttttaccgagagcatgaagggcgttttttctatcagcggctggtggagttcatgacaagtgggccaatccgagcctatatccttgcccacaaagatgccatccaactttggaggacactgatgggacccaccagagtatttcgagcacgctatatagccccagattcaattcgtggaagtttgggcctcactgacacccgaaatactacccatggctcagactccgtggtttccgccagcagagagattgcagccttcttccctgacttcagtgaacagcgctggtatgaggaggaggaaccccagctgcggtgtggtcctgtgcactacagtccagaggaaggtatccactgtgcagctgaaacaggaggccacaaacaacctaacaaaacctag

SEQ ID NO: 63 - protein, 189 aa, Artificial Sequence
  Mouse NME6
MTSILRSPQALQLTLALIKPDAVAHPLILEAVHQQILSNKFLIVRTRELQWKLEDCRRFYREHEGRFFYQRLVEFMTSGPIRAYILAHKDAIQLWRTLMGPTRVFRARYIAPDSIRGSLGLTDTRNTTHGSDSVVSASREIAAFFPDFSEQRWYEEEEPQLRCGPVHYSPEEGIHCAAETGGHKQPNKT

SEQ ID NO: 64 - DNA, 585 bp, Artificial Sequence
  Human NME6
atgacccagaatctggggagtgagatggcctcaatcttgcgaagccctcaggctctccagctcactctagccctgatcaagcctgacgcagtcgcccatccactgattctggaggctgttcatcagcagattctaagcaacaagttcctgattgtacgaatgagagaactactgtggagaaaggaagattgccagaggttttaccgagagcatgaagggcgttttttctatcagaggctggtggagttcatggccagcgggccaatccgagcctacatccttgcccacaaggatgccatccagctctggaggacgctcatgggacccaccagagtgttccgagcacgccatgtggccccagattctatccgtgggagtttcggcctcactgacacccgcaacaccacccatggttcggactctgtggtttcagccagcagagagattgcagccttcttccctgacttcagtgaacagcgctggtatgaggaggaagagccccagttgcgctgtggccctgtgtgctatagcccagagggaggtgtccactatgtagctggaacaggaggcctaggaccagcctga

SEQ ID NO: 65 - protein, 194 aa, Artificial Sequence
  Human NME6
MTQNLGSEMASILRSPQALQLTLALIKPDAVAHPLILEAVHQQILSNKFLIVRMRELLWRKEDCQRFYREHEGRFFYQRLVEFMASGPIRAYILAHKDAIQLWRTLMGPTRVFRARHVAPDSIRGSFGLTDTRNTTHGSDSVVSASREIAAFFPDFSEQRWYEEEEPQLRCGPVCYSPEGGVHYVAGTGGLGPA

SEQ ID NO: 66 - DNA, 525 bp, Artificial Sequence
  Human NME6 1
atgacccagaatctggggagtgagatggcctcaatcttgcgaagccctcaggctctccagctcactctagccctgatcaagcctgacgcagtcgcccatccactgattctggaggctgttcatcagcagattctaagcaacaagttcctgattgtacgaatgagagaactactgtggagaaaggaagattgccagaggttttaccgagagcatgaagggcgttttttctatcagaggctggtggagttcatggccagcgggccaatccgagcctacatccttgcccacaaggatgccatccagctctggaggacgctcatgggacccaccagagtgttccgagcacgccatgtggccccagattctatccgtgggagtttcggcctcactgacacccgcaacaccacccatggttcggactctgtggtttcagccagcagagagattgcagccttcttccctgacttcagtgaacagcgctggtatgaggaggaagagccccagttgcgctgtggccctgtgtga

SEQ ID NO: 67 - protein, 174 aa, Artificial Sequence
  Human NME6 1
MTQNLGSEMASILRSPQALQLTLALIKPDAVAHPLILEAVHQQILSNKFLIVRMRELLWRKEDCQRFYREHEGRFFYQRLVEFMASGPIRAYILAHKDAIQLWRTLMGPTRVFRARHVAPDSIRGSFGLTDTRNTTHGSDSVVSASREIAAFFPDFSEQRWYEEEEPQLRCGPV

SEQ ID NO: 68 - DNA, 468 bp, Artificial Sequence
  Human NME6 2
atgctcactctagccctgatcaagcctgacgcagtcgcccatccactgattctggaggctgttcatcagcagattctaagcaacaagttcctgattgtacgaatgagagaactactgtggagaaaggaagattgccagaggttttaccgagagcatgaagggcgttttttctatcagaggctggtggagttcatggccagcgggccaatccgagcctacatccttgcccacaaggatgccatccagctctggaggacgctcatgggacccaccagagtgttccgagcacgccatgtggccccagattctatccgtgggagtttcggcctcactgacacccgcaacaccacccatggttcggactctgtggtttcagccagcagagagattgcagccttcttccctgacttcagtgaacagcgctggtatgaggaggaagagccccagttgcgctgtggccctgtgtga

SEQ ID NO: 69 - protein, 155 aa, Artificial Sequence
  Human NME6 2
MLTLALIKPDAVAHPLILEAVHQQILSNKFLIVRMRELLWRKEDCQRFYREHEGRFFYQRLVEFMASGPIRAYILAHKDAIQLWRTLMGPTRVFRARHVAPDSIRGSFGLTDTRNTTHGSDSVVSASREIAAFFPDFSEQRWYEEEEPQLRCGPV

SEQ ID NO: 70 - DNA, 528 bp, Artificial Sequence
  Human NME6 3
atgctcactctagccctgatcaagcctgacgcagtcgcccatccactgattctggaggctgttcatcagcagattctaagcaacaagttcctgattgtacgaatgagagaactactgtggagaaaggaagattgccagaggttttaccgagagcatgaagggcgttttttctatcagaggctggtggagttcatggccagcgggccaatccgagcctacatccttgcccacaaggatgccatccagctctggaggacgctcatgggacccaccagagtgttccgagcacgccatgtggccccagattctatccgtgggagtttcggcctcactgacacccgcaacaccacccatggttcggactctgtggtttcagccagcagagagattgcagccttcttccctgacttcagtgaacagcgctggtatgaggaggaagagccccagttgcgctgtggccctgtgtgctatagcccagagggaggtgtccactatgtagctggaacaggaggcctaggaccagcctga

SEQ ID NO: 71 - protein, 175 aa, Artificial Sequence
  Human NME6 3
MLTLALIKPDAVAHPLILEAVHQQILSNKFLIVRMRELLWRKEDCQRFYREHEGRFFYQRLVEFMASGPIRAYILAHKDAIQLWRTLMGPTRVFRARHVAPDSIRGSFGLTDTRNTTHGSDSVVSASREIAAFFPDFSEQRWYEEEEPQLRCGPVCYSPEGGVHYVAGTGGLGPA

SEQ ID NO: 72 - DNA, 585 bp, Artificial Sequence
  Human NME6 sequence optimized for E. coli expression
atgacgcaaaatctgggctcggaaatggcaagtatcctgcgctccccgcaagcactgcaactgaccctggctctgatcaaaccggacgctgttgctcatccgctgattctggaagcggtccaccagcaaattctgagcaacaaatttctgatcgtgcgtatgcgcgaactgctgtggcgtaaagaagattgccagcgtttttatcgcgaacatgaaggccgtttcttttatcaacgcctggttgaattcatggcctctggtccgattcgcgcatatatcctggctcacaaagatgcgattcagctgtggcgtaccctgatgggtccgacgcgcgtctttcgtgcacgtcatgtggcaccggactcaatccgtggctcgttcggtctgaccgatacgcgcaataccacgcacggtagcgactctgttgttagtgcgtcccgtgaaatcgcggcctttttcccggacttctccgaacagcgttggtacgaagaagaagaaccgcaactgcgctgtggcccggtctgttattctccggaaggtggtgtccattatgtggcgggcacgggtggtctgggtccggcatga

SEQ ID NO: 73 - protein, 194 aa, Artificial Sequence; identical to SEQ ID NO: 65
  Human NME6 sequence optimized for E. coli expression

SEQ ID NO: 74 - DNA, 525 bp, Artificial Sequence
  Human NME6 1 sequence optimized for E. coli expression
atgacgcaaaatctgggctcggaaatggcaagtatcctgcgctccccgcaagcactgcaactgaccctggctctgatcaaaccggacgctgttgctcatccgctgattctggaagcggtccaccagcaaattctgagcaacaaatttctgatcgtgcgtatgcgcgaactgctgtggcgtaaagaagattgccagcgtttttatcgcgaacatgaaggccgtttcttttatcaacgcctggttgaattcatggcctctggtccgattcgcgcatatatcctggctcacaaagatgcgattcagctgtggcgtaccctgatgggtccgacgcgcgtctttcgtgcacgtcatgtggcaccggactcaatccgtggctcgttcggtctgaccgatacgcgcaataccacgcacggtagcgactctgttgttagtgcgtcccgtgaaatcgcggcctttttcccggacttctccgaacagcgttggtacgaagaagaagaaccgcaactgcgctgtggcccggtctga

SEQ ID NO: 75 - protein, 174 aa, Artificial Sequence; identical to SEQ ID NO: 67
  Human NME6 1 sequence optimized for E. coli expression

SEQ ID NO: 76 - DNA, 468 bp, Artificial Sequence
  Human NME6 2 sequence optimized for E. coli expression
atgctgaccctggctctgatcaaaccggacgctgttgctcatccgctgattctggaagcggtccaccagcaaattctgagcaacaaatttctgatcgtgcgtatgcgcgaactgctgtggcgtaaagaagattgccagcgtttttatcgcgaacatgaaggccgtttcttttatcaacgcctggttgaattcatggcctctggtccgattcgcgcatatatcctggctcacaaagatgcgattcagctgtggcgtaccctgatgggtccgacgcgcgtctttcgtgcacgtcatgtggcaccggactcaatccgtggctcgttcggtctgaccgatacgcgcaataccacgcacggtagcgactctgttgttagtgcgtcccgtgaaatcgcggcctttttcccggacttctccgaacagcgttggtacgaagaagaagaaccgcaactgcgctgtggcccggtctga

SEQ ID NO: 77 - protein, 155 aa, Artificial Sequence; identical to SEQ ID NO: 69
  Human NME6 2 sequence optimized for E. coli expression

SEQ ID NO: 78 - DNA, 528 bp, Artificial Sequence
  Human NME6 3 sequence optimized for E. coli expression
atgctgaccctggctctgatcaaaccggacgctgttgctcatccgctgattctggaagcggtccaccagcaaattctgagcaacaaatttctgatcgtgcgtatgcgcgaactgctgtggcgtaaagaagattgccagcgtttttatcgcgaacatgaaggccgtttcttttatcaacgcctggttgaattcatggcctctggtccgattcgcgcatatatcctggctcacaaagatgcgattcagctgtggcgtaccctgatgggtccgacgcgcgtctttcgtgcacgtcatgtggcaccggactcaatccgtggctcgttcggtctgaccgatacgcgcaataccacgcacggtagcgactctgttgttagtgcgtcccgtgaaatcgcggcctttttcccggacttctccgaacagcgttggtacgaagaagaagaaccgcaactgcgctgtggcccggtctgttattctccggaaggtggtgtccattatgtggcgggcacgggtggtctgggtccggcatga

SEQ ID NO: 79 - protein, 175 aa, Artificial Sequence; identical to SEQ ID NO: 71
  Human NME6 3 sequence optimized for E. coli expression

SEQ ID NO: 80 - DNA, 27 bp, Artificial Sequence
  Histidine Tag
ctcgagcaccaccaccaccaccactga

SEQ ID NO: 81 - DNA, 36 bp, Artificial Sequence
  Strept II Tag
accggttggagccatcctcagttcgaaaagtaatga

SEQ ID NO: 82 - protein, 35 aa, Artificial Sequence
  PSMGFR N-10 peptide
QFNQYKTEAASRYNLTISDVSVSDVPFPFSAQSGA

SEQ ID NO: 83 - protein, 35 aa, Artificial Sequence
  PSMGFR C-10 peptide
GTINVHDVETQFNQYKTEAASRYNLTISDVSVSDV

SEQ ID NO: 84 - protein, 376 aa, Artificial Sequence
  nucleoside diphosphate kinase 7 isoform a [Homo sapiens] (Hu_7)
MNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 85 - DNA, 759 bp, Artificial Sequence
  Human NME7-X1
atgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattag

SEQ ID NO: 86 - protein, 252 aa, Artificial Sequence [sequence omitted]

SEQ ID NO: 87 - protein, 44 aa, Artificial Sequence
  Mouse MUC1* extracellular domain PSMGFR
GTFSASDVKSQLIQHKKEADDYNLTISEVKVNEMQFPPSAQSRP

SEQ ID NO: 88 - protein, 395 aa, Artificial Sequence
  nucleoside diphosphate kinase 7 (mouse NME7) isoform 1 Mus musculus (Mo_7)
MRACQQGRSSSLVSPYMAPKNQSERFAFIAEWYDPNASLLRRYELLFYPTDGSVEMHDVKNRRTFLKRTKYEDLRLEDLFIGNKVNVFSRQLVLIDYGDQYTARQLGSRKEKTLALIKPDAVSKAGEIIEMINKSGFTITKLRMMTLTRKEAADFHVDHHSRPFYNELIQFITSGPVIAMEILRDDAICEWKRLLGPANSGLSRTDAPGSIRALFGTDGVRNAAHGPDTFASAAREMELFFPSSGGCGPANTAKFTNCTCCIIKPHAISEGMLGKILIAIRDACFGMSAIQMFNLDRANVEEFYEVYKGVVSEYNDMVTELCSGPCVAIEIQQSNPTKTFREFCGPADPEIARHLRPETLRAIFGKTKVQNAVHCTDLPEDGLLEVQYFFKILDN

SEQ ID NO: 89 - protein, 277 aa, Artificial Sequence
  nucleoside diphosphate kinase 7 isoform 2 [Mus musculus] (Mo2-7)
MRACQQGRSSSLVSPYMAPKNQSERFAFIAEWYDPNASLLRRYELLFYPTDGSVEMHDVKNRRTFLKRTKYEDLRLEDLFIGNKVNVFSRQLVLIDYGDQYTARQLGSRKEKTLALIKPDAVSKAGEIIEMINKSGFTITKLRMMTLTRKEAADFHVDHHSRPFYNELIQFITSGPVIAMEILRDDAICEWKRLLGPANSGLSRTDAPGSIRALFGTDGVRNAAHGPDTFASAAREMELFFPSSGGCGPANTAKFTNCTCCIIKPHAISEDLFIHYM

SEQ ID NO: 90 - protein, 213 aa, Artificial Sequence
  Pig Sus scrofa MUC1
MTRDIQAPFFFGLLLLPVLTGEGNKQTNKNLALSLSSQFLQVYKEDGLLGLFYIKFRPGSVLVELILAFQDSAAAHNLKTQFDRLKAEAGTYNLTISEVSVIDAPFPSSAQPGSGVPGWGIALLVLVCILVALAIIYVIALAVCQCRRKNCGQLDIFPTRDAYHPMSEYPTYHTHGRYVPPGSTKRNPYEQVSAGNGGGSLSYSNLAATSANL

SEQ ID NO: 91 - protein, 45 aa, Artificial Sequence
  Pig MUC1* PSMGFR
QDSAAAHNLKTQFDRLKAEAGTYNLTISEVSVIDAPFPSSAQPGS

SEQ ID NO: 92 - protein, 453 aa, Artificial Sequence
  PREDICTED: nucleoside diphosphate kinase 7 [Sus scrofa, Pig] (Pi_7)
MNHSERFVFIAEWYDPNASLFRRYELLFYPGDGSVEMHDVKNHRTFLKRTKYEDLHLEDLFIGNKVNVFSRQLVLIDYGDQYTARQLGSKKEKTLALIKPDAVSKAGEIIEIINKAGFTLTKLKMMTLSRKEATDFHIDHQSRPFLNELIQFITSGPIIAMEILRDDAICEWKKLLGPANSGLARTDAPGSIRAVFGTDGIRNAAHGPDSLSCAAREMELFFPSSGVCGPANTAKFTNCTTCCIVKPHAISEGLLGKILMAIRDAGFEISAMQMFNMDRVNVEEFYEVYKGVVSEYNEMVTEMYFSAPSSSAIWRSPTVLNSLQSDISSRDFSSGPRSIPRSNFYWLTNHLLEMLSLLLLGVHKGVPKEVFVGEAHVSPGCAPVLVGGTLSRVKDRKKENHFSLVFVMLSSVSLPASSRYVKAAKGPQLIKGFSRGRGLLLALNTGCGNCFWL

SEQ ID NO: 93 - protein, 640 aa, Artificial Sequence
  Sheep MUC1
MTPDIQAPFLSLLLLFQVLTVANVTMLTASVSTSPNSTVQVSSTQSSPTSSPTKETSWSTTTTLLRTSSPAPTPTTSPGRDGASSPTSSAAPSPAASSSHDGALSLTGSPAPSPTASPGHGGTLTTTSSPAPSPTASPGHDGASTPTSSPAPSPAASPGHDGALSLTGSPAPSPTASPGHGGTLTTTSSPAPSPTASPGHDGASTPTSSPAPSPAASSSHDGALSLTGSPAPSPPASPGHGGTLTTTSSPAPSPTASPGHGGTLTTTSSPAPSPTASPGHDGASTPTSSPAPTAHSSHDGALTTTGSPAPSPAASPGHDSVPPRATSPAPSPAASPGQHAASSPTSSDISSVTTSSMSSSMVTSAHKGTSSRATTTPVSKGTPSSVPSSETAPTAASHSTRTAAASTSPSTALSTASHPKTSQQLSVQVSLFFLSFRITNLQFNSSLENPQTSYYQELQRSILDVILQTYKQRDFLGLSEIKFRPGSVLVDLTLAFREGTTAELVKAQFSQLEAHAANYSLTISGVSVRDAQFPSSAPSASGVPGWGIALLVLVCVLVALAIIYLIALVVCQCGRKKCEQLDIFPTLGAYHPMSEYSAYHTHGRFVPPGSTKRSPYEEVSAGNGGSNLSYTNLAATSANL

SEQ ID NO: 94 - protein, 45 aa, Artificial Sequence
  Sheep MUC1* extracellular domain PSMGFR
REGTTAELVKAQFSQLEAHAANYSLTISGVSVRDAQFPSSAPSAS

SEQ ID NO: 95 - protein, 395 aa, Artificial Sequence
  PREDICTED: nucleoside diphosphate kinase 7 isoform X1 [Ovis aries, Sheep] (Sh_7)
MNPTFVLLSLERNVTESLGNHSERFVFIAEWFDPNASLFRRYELLFYPGDGSVEMHDVKNHRTFLKRTKYEDLHLEDLFIGNKVNIFSRQLVLLDYGDQYTARQLGSRKEKTLALIKPDAVSKAGEIIEIINKAGFTLTKLKMMTLSRKEATDFHIDHQSRPFLNELIQFITSGPIIAMEILRDDAICEWKRLLGPANSGLARTDAPESIRALFGTDGIKNAAHGPDSFACAAREMELFFPSSGVCGPANTAKFTNCTTCCIVKPHAVSEGLLGKILITIRDAGFEISAMQMFNMDRINVEEFYEVYKGVVSEYNEMVTEMYSGPCVAMEIQQTNPTMTFREFCGPADPEIARHLRPGTLRAIFGKTKIQNAVHCTDLPEDGLLEVQYFFKILDN

SEQ ID NO: 96 - protein, 45 aa, Artificial Sequence
  Crab-eating macaque MUC1* extracellular domain PSMGFR
GTTNVHDVETQFNQRKTEAASRYNLTISDISVRDVPFPFSAQTGA

SEQ ID NO: 97 - protein, 256 aa, Artificial Sequence
  unnamed protein product [Macaca fascicularis] (Ma_7) (sequence incomplete, only NME7A)
MSHSERFVFIAEWYDPNASLLRRYELLFYPGDGSVEMHDVKNHRTFLKRTKYDSLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITSGPVIAMEILRDDAICEWKRLLGPANSGVARTDASGSIRALFGTDGIRNAAHGPDSFASAAREMELFFPSSGGCGPANTAKFTNCTCCIVKPHAVSEVRRNP

SEQ ID NO: 98 - protein, 45 aa, Artificial Sequence; identical to SEQ ID NO: 96
  Rhesus macaque MUC1* extracellular domain PSMGFR

SEQ ID NO: 99 - protein, 376 aa, Artificial Sequence
  Macaca mulatta (Rhesus macaque) (Mm_7)
MSHSERFVFIAEWYDPNASLLRRYELLFYPGDGSVEMHDVKNHRTFLKRTKYDSLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITSGPVIAMEILRDDAICEWKRLLGPANSGVARTDASGSIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPVDPEIARHLRPGTLRAIFGKTKIQNAVHCTDLPEDGLLEVQYFFKILDN

SEQ ID NO: 100 - protein, 484 aa, Artificial Sequence
  Bonobo Pan paniscus MUC1
MTPGVQSPFFLLLLLTVLTATTAPKPATVVTGSGHASSAPGGEKETSATQRSSVPSSTEKNAVSMTSSVLSSHSPGSGSSTTQGQDVTLAPATEPASGSAATWGQDVTSVPVTRPALGSTTPPAHDVTSALDNKPAPGSTAPPAHDVTSAPDTRPAPGSTAPPAHGVTSAPDTRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLRFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 101 - protein, 45 aa, Artificial Sequence; identical to SEQ ID NO: 6
  Bonobo MUC1* extracellular domain PSMGFR

SEQ ID NO: 102 - protein, 376 aa, Artificial Sequence; identical to SEQ ID NO: 84
  Bonobo PREDICTED: nucleoside diphosphate kinase 7 [Pan paniscus, Bonobo] (Bo_7)

SEQ ID NO: 103 - protein, 475 aa, Artificial Sequence
  Pan troglodytes MUC1
MTPGIQSPFFLLLLLTVLTVVTGSGHASSAPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHDVTSAPDTRPAPGSTAPPAHGVTSAPDTRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLRFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 104 - protein, 45 aa, Artificial Sequence; identical to SEQ ID NO: 6
  Chimpanzee MUC1* extracellular domain PSMGFR

SEQ ID NO: 105 - protein, 376 aa, Artificial Sequence
  nucleoside diphosphate kinase 7 [Pan troglodytes, Chimp] (CH_7)
MNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHNMVTEMYSGPCVAMEIQQNNATKTFREFCGPADPEIARHLRPGTLRAIFGKTKIQNAVHCTDLPEDGLLEVQYFFKILDN

SEQ ID NO: 106 - protein, 486 aa, Artificial Sequence
  Gorilla gorilla MUC1
MTPGTQSPFFLLLLLTVLTATTAPKPTTVVTGSGHASSTPGGEKETSATQRSSVPSSTEKNAVSMTSSILSSHSPGSGSSTTQGQDVTPAPATEPASGSAATWGQDVTSVPVTRPALGSTTPPAHDVTSAPDNKPAPGSTTPPAHGVSSAPDTRPAPGSTAPPAHGVTSAPDTRPAPGSTAPPAHVHNVTSASGSASGSASTLVHNGTSARATTTPASKSTPFSIPSHHSDTPTTLANHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVVLAIVYLIALAVCQCRRKNYGQLDIFPVRDTYHPMSEYPTYHTHGRYVPPSSTDRSPYEKVSAGNGGSSLSYTNPAVAATSANL

SEQ ID NO: 107 - protein, 45 aa, Artificial Sequence; identical to SEQ ID NO: 6
  Gorilla MUC1* PSMGFR

SEQ ID NO: 108 - protein, 294 aa, Artificial Sequence
  NME7 [ENSGGOP00000002464], Gorilla gorilla (Go_7)
MNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAARLLGKILMAIRDAGFEISAMQMFNMDRVNVEEFYEVYKGVVTEYHDMVTEMYSGPCVAMEIQQNNATKTFREFCGPADP

SEQ ID NO: 109 - protein, 378 aa, Artificial Sequence
  nucleoside diphosphate kinase 7 [Mus musculus] (Mo_7)
MKTNQSERFAFIAEWYDPNASLLRRYELLFYPTDGSVEMHDVKNRRTFLKRTKYEDLRLEDLFIGNKVNVFSRQLVLIDYGDQYTARQLGSRKEKTLALIKPDAVSKAGEIIEMINKSGFTITKLRMMTLTRKEAADFHVDHHSRPFYNELIQFITSGPVIAMEILRDDAICEWKRLLGPANSGLSRTDAPGSIRALFGTDGVRNAAHSPDTFASAAREMELFFPSSGGCGPANTAKFTNCTCCIIKPHAISEGMLGKILIAIRDACFGMSAIQMFNLDRANVEEFYEVYKGVVSEYNDMVTELCSGPCVAIEIQQSNPTKTFREFCGPADPEIARHLRPETLRAIFGKTKVQNAVHCTDLPEDGLLEVQYFFKILDN

SEQ ID NO: 110 - protein, 340 aa, Artificial Sequence
  nucleoside diphosphate kinase 7 isoform X1 [Mus musculus] (MoX1-7)
MHDVKNRRTFLKRTKYEDLRLEDLFIGNKVNVFSRQLVLIDYGDQYTARQLGSRKEKTLALIKPDAVSKAGEIIEMINKSGFTITKLRMMTLTRKEAADFHVDHHSRPFYNELIQFITSGPVIAMEILRDDAICEWKRLLGPANSGLSRTDAPGSIRALFGTDGVRNAAHGPDTFASAAREMELFFPSSGGCGPANTAKFTNCTCCIIKPHAISEGMLGKILIAIRDACFGMSAIQMFNLDRANVEEFYEVYKGVVSEYNDMVTELCSGPCVAIEIQQSNPTKTFREFCGPADPEIARHLRPETLRAIFGKTKVQNAVHCTDLPEDGLLEVQYFFKILDN

SEQ ID NO: 111 - protein, 256 aa, Artificial Sequence; identical to SEQ ID NO: 97
  unnamed protein product [Macaca fascicularis] (Ma_7) (sequence incomplete, only NME7A)

SEQ ID NO: 112 - protein, 376 aa, Artificial Sequence; identical to SEQ ID NO: 99
  Macaca mulatta (Rhesus macaque) (Mm_7)

SEQ ID NO: 113 - protein, 340 aa, Artificial Sequence
  nucleoside diphosphate kinase 7 b [Pan troglodytes, Chimp] (CHb_7)
MHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHSRPGTLRAIFGKTKIQNAVHCTDLPEDGLLEVQYFFKILDN

SEQ ID NO: 114 - protein, 377 aa, Artificial Sequence
  PREDICTED: nucleoside diphosphate kinase 7 isoform X2 (Shx2_7) [Ovis aries, Sheep]
MNHSERFVFIAEWFDPNASLFRRYELLFYPGDGSVEMHDVKNHRTFLKRTKYEDLHLEDLFIGNKVNIFSRQLVLLDYGDQYTARQLGSRKEKTLALIKPDAVSKAGEIIEIINKAGFTLTKLKMMTLSRKEATDFHIDHQSRPFLNELIQFITSGPIIAMEILRDDAICEWKRLLGPANSGLARTDAPESIRALFGTDGIKNAAHGPDSFACAAREMELFFPSSGVCGPANTAKFTNCTTCCIVKPHAVSEGLLGKILITIRDAGFEISAMQMFNMDRINVEEFYEVYKGVVSEYNEMVTEMYSGPCVAMEIQQTNPTMTFREFCGPADPEIARHLRPGTLRAIFGKTKIQNAVHCTDLPEDGLLEVQYFFKILDN

SEQ ID NO: 115 - protein, 341 aa, Artificial Sequence
  PREDICTED: nucleoside diphosphate kinase 7 isoform X3 [Ovis aries, Sheep] (Shx3_7)
MHDVKNHRTFLKRTKYEDLHLEDLFIGNKVNIFSRQLVLLDYGDQYTARQLGSRKEKTLALIKPDAVSKAGEIIEIINKAGFTLTKLKMMTLSRKEATDFHIDHQSRPFLNELIQFITSGPIIAMEILRDDAICEWKRLLGPANSGLARTDAPESIRALFGTDGIKNAAHGPDSFACAAREMELFFPSSGVCGPANTAKFTNCTTCCIVKPHAVSEGLLGKILITIRDAGFEISAMQMFNMDRINVEEFYEVYKGVVSEYNEMVTEMYSGPCVAMEIQQTNPTMTFREFCGPADPEIARHLRPGTLRAIFGKTKIQNAVHCTDLPEDGLLEVQYFFKILDN

SEQ ID NO: 116 - protein, 630 aa, Artificial Sequence
  Mouse MUC1
MTPGIRAPFFLLLLLASLKGFLALPSEENSVTSSQDTSSSLASTTTPVHSSNSDPATRPPGDSTSSPVQSSTSSPATRAPEDSTSTAVLSGTSSPATTAPVNSASSPVAHGDTSSPATSLSKDSNSSPVVHSGTSSAATTAPVDSTSSPVVHGGTSSPATSPPGDSTSSPDHSSTSSPATRAPEDSTSTAVLSGTSSPATTAPVDSTSSPVAHDDTSSPATSLSEDSASSPVAHGGTSSPATSPLRDSTSSPVHSSASIQNIKTTSDLASTPDHNGTSVTTTSSALGSATSPDHSGTSTTTNSSESVLATTPVYSSMPFSTTKVTSGSAIIPDHNGSSVLPTSSVLGSATSLVYNTSAIATTPVSNGTQPSVPSQYPVSPTMATTSSHSTIASSSYYSTVPFSTFSSNSSPQLSVGVSFFFLSFYIQNHPFNSSLEDPSSNYYQELKRNISGLFLQIFNGDFLGISSIKFRSGSVVVESTVVFREGTFSASDVKSQLIQHKKEADDYNLTISEVKVNEMQFPPSAQSRPGVPGWGIALLVLVCILVALAIVYFLALAVCQCRRKSYGQLDIFPTQDTYHPMSEYPTYHTHGRYVPPGSTKRSPYEEVSAGNGSSSLSYTNPAVVTTSANL

SEQ ID NO: 117 - protein, 553 aa, Artificial Sequence
  Macaca mulatta (rhesus macaque) MUC1, partial
VTGSGHTNSTPGGEKETSATQRSSMPISTKNAVSMTSRLSSHSPVSGSSTTQGQDVTLALATEPATGSATTLGHNVTSAPDTSAAPGSTGPPAGVVTSAPDTSAAPGSTGPPARVVTSAPDTSAAPGSTGPPARVVTSAPDTSAAPGSTGPPARVVTSAPDTSAAPGSTGPPARVVTSAPDTSAAPGSTGPPARVVTSAPDTSAAPGSTGPPARVVTSAPDTSAAPGSTGPPARVVTSAPDTSAAPGSTGPPARVVTSAPGTSAAPGSTAPPGSTAPPAHDVTSASDSASGSASTLVHSTTSARATTTPASKSTPFSIPSHHSDTPTTLASHSTKTDASSTHHSTVPPFTSNHSTSPQLSLGVSFFFLSFHISNLQFNSSLEDPSTNYYQQLQRDISELFLQIYKQGDFLGLSNIMFRPGSVVVQSTLVFREGTTNVHDVETQFNQRKTEAASRYNLTISDISVRDVPFPFSAQTGAGVPGWGIALLVLVCVLVVLAIVYFIALAVCQCRQKNYRQLDIFPARDAYHPMSEYPTYHTHGRYVPAGGTNRSPYE

SEQ ID NO: 118 - protein, 482 aa, Artificial Sequence
  Macaca fascicularis_MUC1 (isoform X1)
MTPGTQSPFFLLLILTVLTAATVPEPTTVVTGSGHTNSTPGGEKETSATQRSSMPISTKNAVSMTSRLSSHSPVSGSSTTQGQDVTLALAMESATGSATTLGHVVTSAPDTSAAPGSTGPPAHVVTSAPDTSAAPGSTGPPAHVVTSAPDTSAAPGSTAPPAHVVTSAPDTSAAPGSTAPPAHDVTSASDSASGSASTLVHSTTSARATTTPASKSTPFSIPSHHSDTPTTLASHSTKTDASSTHHSTVPPFTSSNHSTSPQLSLGVSFFFLSFHISNLQFNSSLEDPSTNYYQQLQRDISELFLQIYKQGDFLGLSNIMFRPGSVVVQSTLVFREGTTNVHDVETQFNQRKTEAASRYNLTISDISVRDVPFPFSAQTGAGVPGWGIALLVLVCVLVVMAIVYFIALAVCQCRQKNYRQLDIFPARDAYHPMSEYPTYHTHGRYAPAGGTNRSPYEEVSAGNGGSSLSYTNPAVAATSANL

SEQ ID NO: 119 - protein, 197 aa, Artificial Sequence
  Sus scrofa MUC1, partial
NSSLEDPTTSYYKDLQRRISELFLQVYKEDGLLGLFYIKFRPGSVLVELILAFQDSAAAHNLKTQFDRLKAEAGTYNLTISEVSVIDAPFPSSAQPGSGVPGWGIALLVLVCILVALAIIYVIALAVCQCRRKNCGQLDIFPTRDAYHPMSEYPTYHTHGRYVPPGSTKRNPYEQVSAGNGGGSLSYSNLAATSANL

SEQ ID NO: 120 - protein, 640 aa, Artificial Sequence; identical to SEQ ID NO: 93
  Ovis aries MUC1 (Sheep)

(19) United States
(12) Patent Application Publication
(10) Pub. No.: US 2018/0235193 A1
(43) Pub. Date: Aug. 23, 2018

(54) METHOD OF STEM CELL-BASED ORGAN AND TISSUE GENERATION

(71) Applicant: MINERVA BIOTECHNOLOGIES CORPORATION, Waltham, MA (US)

(72) Inventors: Cynthia Bamdad, Waltham, MA (US); Benoit Smagghe, Waltham, MA (US); Mark Carter, Waltham, MA (US); Andrew Stewart, Waltham, MA (US)

(73) Assignee: MINERVA BIOTECHNOLOGIES CORPORATION, Waltham, MA (US)

(21) Appl. No.: 15/740,654

(22) PCT Filed: Jul. 1, 2016
(86) PCT No.: PCT/US16/40880

§ 371 Date: Dec. 28, 2017

Related U.S. Application Data
(60) Provisional application No. 62/187,638, filed on Jul. 1, 2015.
(60) Provisional application No. 62/234,035, filed on Sep. 29, 2015.

(51) Int. Cl. A01K 67/027; A61K 35/545

(52) U.S. Cl. CPC A01K 67/0276; C07K 16/3092; A01K 2227/10; A61K 35/545

(57) ABSTRACT

The present application discloses a method for generating human tissues in a non-human animal comprising: (i) generating human naïve state stem cells and injecting them into a blastocyst or embryo of a non-human animal such that a chimeric animal is generated; (ii) harvesting human tissues, organs, cells or factors secreted by or made in the human tissues or cells from the chimeric animal; and (iii) transplanting or administering the harvested material into a human resulting in generation of human tissues in a non-human animal.

### BACKGROUND OF THE INVENTION

### 1. Field of the Invention

The present application relates to methods of creating human tissue in non-human animals. The present application also relates to testing for drugs on the non-human animal as it relates to human tissue present in the non-human animal. The present application also relates to methods of treating patients with the human tissue or organs obtained from the non-human animal. The present application also relates to methods of generating transgenic animals that would enable incorporation of human cells and tissues.

### 2. General Background and State of the Art

Currently, there is considerable research in the area of making chimeric animals by inserting stem cells into a developing embryo or blastocyst. The end goal is to be able to generate human organs or tissue in an animal such as a cow, sheep or pig, wherein the resultant organs or tissues would be used for transplant into humans. The donor stem cells could be from a patient in need of a new liver, heart or the like. If successful, the technology would replace the need for an organ donor; currently most patients die before a suitable organ donor is identified.

Technology now exists for: 1) making a knockout animal that will not develop a heart, a pancreas, lungs or other organ or tissue; 2) making a chimeric animal by injecting donor stem cells into a morula or blastocyst of another; 3) making a chimeric animal wherein the fertilized or unfertilized egg has had gene(s) knocked out such that they no longer have the capacity to form a particular organ or tissue, but where the donor stem cells do have the capacity to form that particular organ or tissue, so chimeric animal may have up to 100% DNA of the donor stem cells in the targeted organ. These techniques have been shown to work for across species applications such as mouse to rat and rat to mouse. The size of the organ generated depends on the size of the recipient animal. However, rat and mouse are close species.

Ethical issues may impede the generation of non-human primate-human chimeras. Although more distant mammal-human chimeras may overcome certain ethical issues, attempts to generate non-primate species-human chimeras have thus far failed.

One problem is that, at least in the rodent examples, only naïve state stem cells were able to incorporate into the morula or inner cell mass and form chimeras. Primed state stem cells were not able to.

The present invention solves the problem by providing a method for generating human stem cells in the naïve state for the generation of non-human-human chimeric animals and also for providing an environment suitable for the growth of pluripotent human stem cells in a non-human environment. The present invention solves the problem by providing a method for “humanizing” host animals by genetically altering them such that they express human factors that enable growth and expansion of the human cells in the non-human host animal.

### SUMMARY OF THE INVENTION

I. In one aspect, the present invention is directed to a method of testing for efficacy or toxicity of a potential drug agent in a chimeric animal that expresses some human DNA or some human tissues. In this method, an animal that expresses some human DNA or tissues is generated by introducing human naïve state stem cells into a non-human cell or cells. In one aspect the non-human cell is an egg, in another aspect it is a fertilized egg, in another aspect, the cells are a morula, blastocyst or embryo. For ethical concerns or other reasons, it may be advantageous to generate chimeric animals wherein the integrating naïve state stem cells are also non-human, but of a different species than the recipient cell, cells, morula, blastocyst or embryo. In the method above, the agent that maintains stem cells in the naïve state or reverts primed stem cells to the naïve state may be an NME protein, 2i, 5i, or other cocktails of inhibitors, chemicals, or nucleic acids. The NME protein may be NME1 dimer, NME7 monomer, NME7-AB, NME7-X1, NME6 dimer, or bacterial NME.

The non-human mammal may be a rodent, such as a mouse or rat, primate, including macaque, rhesus monkey, ape, chimp, bonobo and the like, or a domestic animal including pig, sheep, bovine, and the like. The chimeric animal may have a genetic disorder, have an induced disease, or a cancer that may be spontaneously generated or implanted from cells derived from a human being.

In the method above, the non-human animal may be transgenic, wherein the animal expresses human MUC1 or MUC1* or NME protein in the germ cells or somatic cells, wherein the germ cells and somatic cells contain a recombinant human MUC1 or MUC1* or NME gene sequence introduced into said animal. The gene expressing the human MUC1 or MUC1* or NME protein may be under control of an inducible promoter. The promoter may be inducibly responsive to a naturally occurring protein in the non-human animal or an agent that can be administered to the animal before, after or during development. Alternatively, the non-human animal may be transgenic, wherein the animal expresses its native sequence MUC1 or MUC1* or NME protein in the germ cells or somatic cells, wherein the germ cells and somatic cells contain a recombinant native species MUC1 or MUC1* or NME gene sequence introduced into said mammal. The NME species can be NME7, NME7-X1, NME1, NME6 or a bacterial NME.

In the method above, the agent that maintains stem cells in the naïve state or reverts primed stem cells to the naïve state may be an NME protein, 2i, 5i, or other cocktails of inhibitors, chemicals, or nucleic acids. The NME protein may be NME1 dimer, NME7 monomer, NME7-AB, NME7-X1, NME6 dimer, or bacterial NME.

In this method, the agent may suppress expression of MBD3, CHD4, BRD4 or JMJD6. The agent may be siRNA made against MBD3, CHD4, BRD4 or JMJD6, or siRNA made against any gene that encodes a protein that upregulates expression of MBD3, CHD4, BRD4 or JMJD6. The cancer stem cell may be characterized by increased expression of CXCR4 or E-cadherin (CDH1) compared with cancer cells or normal cells.

In another aspect, the invention is directed to a method for generating tissue from xenograft in a non-human mammal, comprising: (i) generating a transgenic non-human mammal, wherein the mammal expresses human MUC1 or MUC1* or NME protein in the germ cells and somatic cells, wherein the germ cells and somatic cells contain a recombinant human MUC1 or MUC1* or NME gene sequence introduced into said mammal, wherein the expression of the gene sequence may be under control of an inducible and repressible regulatory sequence; (ii) transferring stem cells or progenitor cells that are xenogeneic in origin to the non-human mammal such that the gene may be induced to be expressed so as to multiply the number of stem or progenitor cells; and (iii) repressing the gene expression so as to generate tissue from the xenografted stem cells.

In this method, in step (iii), the gene expression repression may be carried out by contacting the stem cells with a tissue differentiation factor, or in step (iii) the gene expression repression may be carried out naturally in the mammal in response to naturally produced host tissue differentiation factor. The transferred cells may be human. The tissue may be an organ. The NME protein may be NME7, NME7-AB, NME7-X1, NME1, NME6, or bacterial NME. The animal may be a mammal, a rodent, a primate or domesticated animal such as a pig, sheep, or bovine species.

II. In other aspects, the present invention is directed to making animals having at least some human cells or cells in which at least some of the DNA is of human origin. Such animals would grow human tissue, tissue containing some human cells or cells containing some human DNA for the generation of human or human-like tissue. In other cases such animals would grow organs comprising at least some human cells. In other cases such animals would grow organs comprised entirely of human cells. In yet other cases, host animals can be genetically or molecularly manipulated even after development to grow human limbs. Limbs, nerves, blood vessels, tissues, organs, or factors made in them, or secreted from them, would then be harvested from the animals and used for a multitude of purposes including but not limited to: 1) transplant into humans; 2) administration into humans for medicinal benefit, including anti-aging; and 3) scientific experiments including drug testing and disease modeling.

In one aspect, the invention is directed to a method for generating human tissues in a non-human animal comprising: (i) generating human naïve state stem cells and injecting them into a fertilized egg, morula, blastocyst or embryo of a non-human animal such that a chimeric animal is generated; (ii) harvesting human tissues, organs, cells or factors secreted by or made in the human tissues or cells from the chimeric animal; and (iii) transplanting or administering the harvested material into a human. The naïve state stem cells may be generated using NME7, NME7-AB, NME7-X1 or dimeric NME1. The naïve stem cells may be iPS cells that have been reprogrammed in a medium containing NME7, NME7-AB, NME7-X1 or dimeric NME1. Or, the naïve stem cells may be embryonic stem cells that have been cultured in a medium containing NME7, NME7-AB, NME7-X1 or dimeric NME1. The non-human cells of the blastocyst or embryo may have been genetically altered. And the genetic alteration may result in the host animal being unable to generate a certain tissue or organ. The genetic alteration may be to make the non-human animal express human molecules that facilitate or enhance the incorporation or growth of human stem or progenitor cells in the non-human host animal. Further, the agent that maintains stem cells in the naïve state or reverts primed stem cells to the naïve state may be an NME protein, 2i, 5i, chemical, or nucleic acid. The NME protein may be NME1 dimer, NME7 monomer, NME7-AB, NME6 dimer, or bacterial NME, or NME7-X1. The non-human animal may be a rodent, mouse, rat, pig, sheep, non-human primate, macaque, chimpanzee, bonobo, gorilla or any non-human mammal. In one aspect of the invention, the non-human animal is chosen for its high sequence homology to human NME protein, especially human NME7-AB or NME7-X1 or high sequence homology to human MUC1* extracellular domain. In some cases, the NME protein may be present in serum-free media as the single growth factor.

One test of whether or not a chimeric animal can be generated is if stem cells from a first species are able to incorporate into the inner cell mass (ICM) of a second species. Chimeric animals are more readily generated when the two different species are closely related, for example two rodents. We injected human naïve state stem cells into a mouse morula and showed they incorporated into the inner cell mass. In a specific example, human naïve state stem cells that had been generated in human NME7-AB, then cultured in human, were injected into a mouse morula 2.5 days after fertilization of the egg. This is before the inner cell mass forms. Forty-eight (48) hours later, the morula was analyzed and such analysis showed that the human stem cells had incorporated into the inner cell mass, indicating that a chimeric animal will develop.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 shows a graph of RT-PCR measurements of the expression of master pluripotency regulator genes Oct4 and Nanog in somatic fibroblast, ibb′ cells that were cultured in either normal fibroblast growth media, or a serum free minimal media to which was added a human recombinant NM23, also called NME1 dimers, NME7-AB, or HSP593 bacterial NME1 dimers. ‘+ROCi’ refers to Rho kinase inhibitor that was added to some cells to make them adhere to the surface.

FIG. 2 shows a graph of RT-PCR measurements of the expression of genes that code for chromatin rearrangement factors BRD4, JMJD6, MBD3 and CHD4, in somatic fibroblast, Ibb′ cells that were cultured in either normal fibroblast growth media, or a serum free minimal media to which was added a human recombinant NM23, also called NME1 dimers, NME7-AB, or HSP593 bacterial NME1 dimers. ‘+ROCi’ refers to Rho kinase inhibitor that was added to some cells to make them adhere to the surface.

FIG. 3 shows a graph of RT-PCR measurements of the expression of pluripotency genes OCT4 and NANOG, chromatin rearrangement factors BRD4, JMJD6, MBD3 and CHD4, and NME1 and NME7 in somatic fibroblast, Ibb′ cells that were cultured in either normal fibroblast growth media, or a serum free minimal media to which was added a human recombinant NM23, also called NME1 dimers, NME7-AB, or HSP593 bacterial NME1 dimers. ‘+ROCi’ refers to Rho kinase inhibitor that was added to some cells to make them adhere to the surface.

FIG. 4A-4B shows photographs of human embryonic stem cells with pluripotent morphology wherein the stem cells have been cultured in a serum-free minimal media with recombinant human NME1 dimers as the only growth factor added. FIG. 4A shows photograph taken at 4× magnification, and FIG. 4B shows photograph taken at 20× magnification.

FIG. 5A-5C shows photographs of human embryonic stem cells with pluripotent morphology wherein the stem cells have been cultured in a serum-free minimal media with recombinant human NME7-AB as the only growth factor added. FIG. 5A shows photograph taken at 4× magnification, FIG. 5B shows photograph taken at 10× magnification, and FIG. 5C shows photograph taken at 20× magnification.

FIG. 6A-6B shows graph of HRP signal from ELISA sandwich assay showing NME7-AB dimerizes MUC1* extra cellular domain peptide. FIG. 6A shows an amount of NME7-AB that bound to a surface coated with the MUC1* extracellular domain peptide PSMGFR, and FIG. 6B shows a second MUC1* peptide binding to a second site on the bound NME7-AB.

FIG. 7 shows a graph of RT-PCR measurement of the expression levels of transcription factors BRD4 and co-factor JMJD6 in the earliest stage naïve human stem cells compared to the later stage primed stem cells.

FIG. 8 shows photographs of human fibroblast cells after 18 days in culture in a serum-free media containing human NME1 in dimer form at 4× magnification.

FIG. 9 shows photographs of human fibroblast cells after 18 days in culture in a serum-free media containing human NME1 in dimer form at 20× magnification.

FIG. 10 shows photographs of human fibroblast cells after 18 days in culture in a serum-free media containing human NME7-AB at 4× magnification.

FIG. 11 shows photographs of human fibroblast cells after 18 days in culture in a serum-free media containing human NME7-AB at 20× magnification.

FIG. 12 shows photographs of human fibroblast cells after 18 days in standard media without NME protein at 4× magnification.

FIG. 13 shows photographs of human fibroblast cells after 18 days in standard media without NME protein at 20× magnification.

FIG. 14A-14C shows a cartoon of mechanistic model of inventors' discovery of how human stem cells limit their self-replication. FIG. 14A shows that NME7-AB is secreted from naive stem cells and how NME7-AB dimerizes MUC1* growth factor receptor as a monomer, but is later suppressed by BRD4 while co-factor JMJD6 upregulates NME1, FIG. 14B shows that NME1 is secreted by a later naive state stem cell, but only binds to MUC1* as a dimer, and FIG. 14C shows that as number of stem cells increases so does concentration of NME1 causing it to form hexamers that do not bind to MUC1* and they induce differentiation.

FIG. 15A-15B shows photographs of human blastocysts that were stained with an antibody the inventors developed that recognizes NME7-AB and NME7-X1. FIG. 15A shows a Day 3 blastocyst wherein every cell stains positive for presence of NME7-AB or NME7-X1, and FIG. 15B shows a Day 5 blastocyst wherein only the naive cells of the inner cell mass stain positive for presence of NME7-AB or NME7-X1.

FIG. 16A-16B shows photographs of Western blot gels from a co-immunoprecipitation experiment in which human naive state induced pluripotent stem (iPS) cells and embryonic stem (ES) cells were lysed and an antibody against the cytoplasmic tail of MUC1 (Ab5) was used to co-immunoprecipitate species that bind to MUC1. The immunoprecipitates were then assayed by Western blot. FIG. 16A shows a photograph of the Western blot that was probed with an anti-NME7 antibody and shows two NME7 species, one with molecular weight of 30 kDa and the other 33 kDa, bound to MUC1, whereas full-length NME7 in crude cell lysate has molecular weight of 42 kDa, and FIG. 16B shows a photograph of the Western blot wherein the gel of FIG. 16A was stripped and re-probed with an anti-MUC1* extracellular domain antibody, showing that NME7-AB or NME7-X1 bound to the cleaved form of MUC1 called MUC1* that runs with a molecular weight of 17-25 kDa, depending on glycosylation.

FIG. 17 shows a cartoon of the inventive method for growing human stem cell in the naive state using NME7-AB as the only growth factor on an adhesion surface of MNC3 anti-MUC1* extracellular domain antibody. When it is desired to induce differentiation, a synthetic MUC1* extracellular domain peptide is added to bind up all the NME7-AB.

FIG. 18A-18C shows heat maps from an RNA SEQ experiment in which human embryonic stem (ES) cells that were derived in FGF, and thus in the primed state, were reverted to a less mature naive state by culturing in NME7-AB or in NME1 dimers. FIG. 18A shows heat map of parent FGF-cultured ES cells, FIG. 18B shows heat map of parent cells cultured for 10 passages in NME7-AB, and FIG. 18C shows heat map of parent cells cultured for 10 passages in NME1 dimers.

FIG. 19A-19B shows photographs of human embryonic stem (ES) cells stained with an antibody that binds to tri-methylated Lysine 27 on Histone 3, wherein the presence of red foci indicates that the second X chromosome of female source stem cells has been inactivated, XaXi, which is a sign that the cells are primed state and have initiated early differentiation and have made some cell fate decisions; the presence of a red cloud or absence of red staining indicates that both X chromosomes are still active, XaXa, and thus no differentiation decisions have been made yet, so are naive. FIG. 19A shows photographs of female source stem cells that have been cultured for at least 10 passages in FGF media, and FIG. 19B shows photographs of the same cells that have been cultured for 10 passages in NME7-AB media.

FIG. 20A-20B shows photographs of human pluripotent stem cells in culture in NME7-AB media over a MNC3 anti-MUC1* antibody surface at two time points showing a 10-20-fold expansion rate over a period of four days which is a much faster growth rate than primed state stem cells and is another indication that stem cells cultured in NME7-AB are in naive state. FIG. 20A shows the stem cells at Day 4, and FIG. 20B shows stem cells at Day 4.

FIG. 21A-21C shows photographs of fibroblasts being reprogrammed to become pluripotent stem cells using Yamanaka master reprogramming factors OCT4, NANOG, KLF4 and c-Myc delivered using Sendai viral transduction wherein newly reprogrammed induced pluripotent stem cells are visualized via staining with alkaline phosphatase. FIG. 21A shows reprogramming being done in FGF media over MEF feeder cells, FIG. 21B shows reprogramming being done in mTeSR media over Matrigel, and FIG. 21C shows reprogramming being done in NME7-AB media over MNC3 anti-MUC1* antibody surface.

FIG. 22A-22D shows photographs of human iPS cells that carry a fluorescent marker, yellow cells, which have been derived and cultured in NME7-AB, injected into a mouse blastocyst at Day 2.5 then imaged 48 hours later at Day 4.5 and show that human iPS cells cultured in NME7-AB have ability to integrate into the inner cell mass of mouse embryo. FIG. 22A shows fluorescent microscopy photographs of Clone E human iPS cells (yellow) that have found their way into the mouse embryo's inner cell mass that contains the naive stem cells, FIG. 22B is the same photograph but with another fluorescent channel opened to allow imaging of DAPI, blue, to show all the cells and showing that the NME7-AB cells have only integrated into inner cell mass plus a whiff in the trophectoderm that will develop into placenta, FIG. 22C shows fluorescent microscopy photographs of Clone R human iPS cells (yellow) that have found their way into the mouse embryo's inner cell mass that contains the naive stem cells, and FIG. 22D is the same photograph but with another fluorescent channel opened to allow imaging of DAPI to show all the cells and showing that the NME7-AB cells have only integrated into inner cell mass plus a whiff in the trophectoderm that will develop into placenta.

FIG. 23A-23J shows confocal images of NME7-AB generated human iPS cells, clone E, that were injected into a Day 2.5 mouse morula. Staining for human Tra 1-81 (red) 48 hours later shows incorporation of the NME7-AB human stem cells into the inner cell mass of the mouse morula. FIGS. 23A, 23C, 23E, 23G, and 23I are fluorescent images. FIGS. 23B, 23D, 23F, 23H, and 23J are bright field images.

FIG. 24A-24J shows confocal images of NME7-AB generated human iPS cells, clone R, that were injected into a Day 2.5 mouse morula. Staining for human Tra 1-81 (red) 48 hours later shows incorporation of the NME7-AB human stem cells into the inner cell mass of the mouse morula. FIGS. 24A, 24C, 24E, 24G, and 24I are fluorescent images. Arrows point to incorporation of naive state NME7-AB human stem cells into the mouse morula, indicating forming of a chimeric animal. FIGS. 24B, 24D, 24F, 24H, and 24J are bright field images.

FIG. 25A-25D shows confocal images of FGF generated human iPS cells that were injected into a Day 2.5 mouse morula. Staining for human Tra 1-81 (red) and staining for trophectoderm (green) 48 hours later shows some scattered human stem cells but no incorporation of the primed state human stem cells into the inner cell mass of the mouse morula, indicating failure to start to form a chimeric animal. FIGS. 25A and 25C are fluorescent images, and FIGS. 25B and 25D are bright field images.

FIG. 26A-26D shows confocal images of human iPS cells that were cultured in 50% NME7-AB media and 50% KSOM, Potassium Simplex Optimized Medium, then injected into a Day 2.5 mouse morula. Fluorescent as well as bright field images were taken 48 hours later. Staining for human Tra 1-81 (red) and staining for trophectoderm (green) 48 hours later shows minimal number of human cells, if any, integrating and no incorporation of the primed state human stem cells into the inner cell mass of the mouse morula, indicating failure to start to form a chimeric animal. FIGS. 26A, 26C, 26^{E} and 26G are fluorescent images, and FIGS. 26B, 26D, 26F and 26H include DAP staining.

FIG. 27A-27F shows confocal fluorescent images of NME7-AB generated human iPS cells, clone E, that were transfected with a fluorescent label, tdtomato, so they would self-fluoresce red. They were injected into a Day 2.5 mouse morula. Images were taken 48 hours later and show integration of the NME7-AB human cells into the inner cell mass of the mouse morula. FIGS. 27A, 27B, 27C show NME7-AB human cells in red, the trophectoderm of the mouse cells in green and FIGS. 27D, 27E, and 27F show NME7-AB human cells in red, the trophectoderm of the mouse cells in green and all cell nuclei in blue from DAPI staining.

FIG. 28A-28J shows confocal fluorescent images of NME7-AB generated human iPS cells, clone E, that were transfected with a fluorescent label, tdtomato, so they would self-fluoresce red. They were injected into a Day 2.5 mouse morula and images were taken 48 hours later. The photographs show integration of the NME7-AB human cells into the inner cell mass of the mouse morula. FIGS. 28A, 28C, 28E, 28G, and 28I show NME7-AB human cells in red, the trophectoderm of the mouse cells in green, and FIGS. 28B, 28D, 28F, and 28H show NME7-AB human cells in red, the trophectoderm of the mouse cells in green and all cell nuclei in blue from DAPI staining.

FIG. 29A-29J shows confocal fluorescent images of NME7-AB generated human iPS cells, clone R, that were transfected with a fluorescent label, tdtomato, so they would self-fluoresce red. They were injected into a Day 2.5 mouse morula and images were taken 48 hours later. The photographs show integration of the NME7-AB human cells into the inner cell mass of the mouse morula. Arrows indicate incorporation of the human cells into the mouse inner cell mass. FIGS. 29A, 29C, 29E, 29G, 29I show NME7-AB human cells in red, the trophectoderm of the mouse cells in green and FIGS. 29B, 29D, 29F, and 29H show NME7-AB human cells in red, the trophectoderm of the mouse cells in green and all cell nuclei in blue from DAPI staining.

FIG. 30A-30J shows confocal fluorescent images of NME7-AB generated human iPS cells, clone R, that were transfected with a fluorescent label, tdtomato, so they would self-fluoresce red. They were injected into a Day 2.5 mouse morula. Images were taken 48 hours later. The photographs show integration of the NME7-AB human cells into the inner cell mass of the mouse morula. Arrows indicate incorporation of the human cells into the mouse inner cell mass. FIGS. 30A, 30C, 30E, 30G, and 30I show NME7-AB human cells in red, the trophectoderm of the mouse cells in green and FIGS. 30B, 30D, 30F, and 30H show NME7-AB human cells in red, the trophectoderm of the mouse cells in green and all cell nuclei in blue from DAPI staining.

FIG. 31A-31F shows photographs of control plates for experiment demonstrating generation of non-human primate induced pluripotent stem cells. In these control experiments, fibroblasts from crab-eating macaques were cultured but the core pluripotency genes were not transduced. Images show the morphology of fibroblasts not of stem cells. FIGS. 31A and 31D were photographed at 4× magnification, FIGS. 31B and 31E were photographed at 10× magnification, and FIGS. 31C and 31F were photographed at 20× magnification,

FIG. 32A32-C shows Day 6 photographs of fibroblasts from crab-eating macaques that have been reprogrammed into induced pluripotent stem (iPS) cells by culturing in an NME7 media, in this case NME7-AB, over a surface of anti-MUC1* antibody, in this case MN-C3 and by transducing the cells with core pluripotency genes, in this case Oct4, Sox2, Klf4, and c-Myc. 50,000 fibroblasts from crab-eating macaques were plated per well of a 6-well plate. Emerging colonies with stem-like morphology are circled. FIG. 32A was photographed at 4× magnification, FIG. 32B was photographed at 10× magnification, and FIG. 32C was photographed at 20× magnification.

FIG. 33A-33F shows Day 6 photographs of fibroblasts from crab-eating macaques that have been reprogrammed into induced pluripotent stem (iPS) cells by culturing in an NME7 media, in this case NME7-AB, over a surface of anti-MUC1* antibody, in this case MN-C3 and by transducing the cells with core pluripotency genes, in this case Oct4, Sox2, Klf4, and c-Myc. 100,000 fibroblasts from crab-eating macaques were plated per well of a 6-well plate. Emerging colonies with stem-like morphology are circled. FIGS. 33A and 33D were photographed at 4× magnification, FIGS. 33B and 33E were photographed at 10× magnification, and FIGS. 33C and 33F were photographed at 20× magnification.

FIG. 34A-34F shows Day 14 photographs of fibroblasts from crab-eating macaques that have been reprogrammed into induced pluripotent stem (iPS) cells by culturing in an NME7 media, in this case NME7-AB, over a surface of anti-MUC1* antibody, in this case MN-C3 and by transducing the cells with core pluripotency genes, in this case Oct4, Sox2, Klf4, and c-Myc. Stem cell colonies are clearly visible. FIGS. 34A, 34B, and 34C were photographed at 10× magnification, FIGS. 34D, 34E, and 34F were photographed at 20× magnification, FIGS. 34A and 34D show results from 24,000 macaque fibroblasts being plated, FIGS. 34B and 34E show results from 6,000 macaque fibroblasts being plated, and FIGS. 34C and 34F show results from 12,000 macaque fibroblasts being plated.

FIG. 35A-35D shows photographs of rhesus macaque embryonic stem (ES) cells being proliferated in a serum-free media containing NME7-AB as the only growth factor on Day 1 of the second passage in NME7-AB media on an anti-MUC1* antibody surface. Embryonic stem cell colonies are clearly visible. FIGS. 35A and 35B were photographed at 4× magnification, and FIGS. 35C and 35D were photographed at 10× magnification,

FIG. 36A-36D shows photographs of rhesus macaque embryonic stem (ES) cells being proliferated in a serum-free media containing NME7-AB as the only growth factor on Day 3 of the second passage in NME7-AB media on an anti-MUC1* antibody surface. Embryonic stem cell colonies are clearly visible. FIGS. 36A and 36B were photographed at 4× magnification, and FIGS. 36C and 36D were photographed at 10× magnification.

FIG. 37A-37B shows photographs of rhesus macaque embryonic stem (ES) cells being proliferated in a serum-free media containing NME7-AB as the only growth factor on Day 1, passage 3. FIG. 37A shows scale bar at 1000 um and FIG. 37B shows scale bar at 400 um.

FIG. 38A-38H shows photographs of rhesus macaque embryonic stem (ES) cells being proliferated in a serum-free media containing NME7-AB as the only growth factor on Day 4, passage 3. FIGS. 38A, 38C, 38F were photographed at 4×, FIGS. 38B, 38D, 38G were photographed at 10×, and FIGS. 38E and 38H were photographed at 20×.

FIG. 39A-39C shows Day 14 photographs of fibroblasts from rhesus macaques that have been reprogrammed into induced pluripotent stem (iPS) cells by culturing in an NME7 media, in this case NME7-AB, over a surface of anti-MUC1* antibody, in this case MN-C3 and by transducing the cells with core pluripotency genes, in this case Oct4, Sox2, Klf4, and c-Myc on Day 14 post transduction of pluripotency genes. FIG. 39A was photographed at 4×, FIG. 39B was photographed at 10×, and FIG. 39C was photographed at 20×.

FIG. 40A-40C shows photographs of serial passaging of fibroblasts from rhesus macaques that have been reprogrammed into induced pluripotent stem (iPS) cells by culturing in an NME7 media, in this case NME7-AB, over a surface of anti-MUC1* antibody, in this case MN-C3 and by transducing the cells with core pluripotency genes, in this case Oct4, Sox2, Klf4, and c-Myc. FIG. 40A was photographed at 4×, FIG. 40B was photographed at 10×, and FIG. 40C was photographed at 20×.

FIG. 41A-41D shows photographs of Day 16 derivation of macaque iPS cells in NME7-AB media by transducing the cells with core pluripotency genes, in this case Oct4, Sox2, Klf4, and c-Myc under identical conditions except that in one case the macaque fibroblasts were plated onto MNC3 anti-MUC1* antibody surface and in the other case onto mouse embryonic feeder cells, MEFs. It is clearly observed that iPS derivation of non-human primates is more efficient when plated in the absence of mouse feeder cells. FIGS. 41A and 41B were plated onto MNC3 antibody surface and FIGS. 41C and 41D were plated onto mouse feeder cells.

FIG. 42A-42B shows Day 14 photographs of a fibroblasts from rhesus macaques that have been reprogrammed into induced pluripotent stem (iPS) cells by culturing in an NME7 media, in this case NME7-AB, over a surface of MEFs and by transducing the cells with core pluripotency genes, in this case Oct4, Sox2, Klf4, and c-Myc. FIG. 42A was photographed at 10× magnification, and FIG. 42B was photographed at 20× magnification.

FIG. 43A-43E shows photographs of NME7-AB induced pluripotent stem cells from rhesus macaques over a surface of anti-MUC1* antibody, in this case MN-C3 on Day 3, passage 1. FIG. 43A was photographed at 4× magnification, FIG. 43B was photographed at 10× magnification, FIG. 43C was photographed at 20× magnification, and FIGS. 43D and 43E were photographed at 40× magnification.

FIG. 44A-44F shows photographs of serial passaging of NME7-AB induced pluripotent stem cells from rhesus macaques over a surface of anti-MUC1* antibody, in this case MN-C3 on Day 1, passage 2. As is clearly visible, stem cell colonies are present. FIGS. 44A and 44D were photographed at 10× magnification, FIGS. 44B and 44E were photographed at 20× magnification, and FIGS. 44C and 44F were photographed at 40× magnification.

FIG. 45A-45H shows photographs of serial passaging of NME7-AB induced pluripotent stem cells derived from rhesus macaque fibroblasts over a surface of mouse feeder cells, MEFs, on Day 2 of passage 2. FIGS. 45A and 45E were photographed at 4× magnification, FIGS. 45B and 45F were photographed at 10× magnification, FIGS. 45C and 45G were photographed at 20× magnification, and FIGS. 45D and 45H were photographed at 40× magnification.

FIG. 46A-46H shows photographs of serial passaging of NME7-AB induced pluripotent stem cells from rhesus macaques over mouse feeder cells, MEFs, on Day 3 of passage 2. FIGS. 46A and 46E were photographed at 4× magnification, FIGS. 46B and 46F were photographed at 10× magnification, FIGS. 46C and 46G were photographed at 20× magnification, and FIGS. 46D and 46H were photographed at 40× magnification.

FIG. 47A-47G shows photographs of serial passaging of NME7-AB induced pluripotent stem cells from rhesus macaques over mouse feeder cells, MEFs, on Day 1 and on Day 2 of passage 3. FIGS. 47A, 47B, 47C and 47D were photographed on Day 1 of passage 3, FIGS. 47E, 47F, 47G were photographed on Day 2 of passage 3, FIGS. 47A and 47E were photographed at 4× magnification, FIGS. 47B and 47F were photographed at 10× magnification, FIGS. 47C and 47G were photographed at 20× magnification, and FIG. 47D was photographed at 40× magnification.

FIG. 48A-48D shows photographs of serial passaging of NME7-AB induced pluripotent stem cells from rhesus macaques mouse feeder cells, MEFs, on Day 3, passage 4. FIG. 48A was photographed at 4× magnification, FIGS. 48B and 48C were photographed at 10× magnification, and FIG. 48D was photographed at 20× magnification.

### DETAILED DESCRIPTION OF THE PREFERRED EMBODIMENTS

### Definitions

As used herein, the “MUC1*” extra cellular domain is defined primarily by the PSMGFR sequence (GTINVHDVETQFNQYKTEAASRYNLTISDVSVSDVPFPFSAQSGA (SEQ ID NO:6)). Because the exact site of MUC1 cleavage depends on the enzyme that clips it, and that the cleavage enzyme varies depending on cell type, tissue type or the time in the evolution of the cell, the exact sequence of the MUC1* extra cellular domain may vary at the N-terminus.

As used herein, the term “PSMGFR” is an acronym for Primary Sequence of MUC1 Growth Factor Receptor as set forth as GTINVHDVETQFNQYKTEAASRYNLTISDVSVSDVPFPFSAQSGA (SEQ ID NO:6). In this regard, the “N-number” as in “N-10 PSMGFR”, “N-15 PSMGFR”, or “N-20 PSMGFR” refers to the number of amino acid residues that have been deleted at the N-terminal end of PSMGFR. Likewise “C-number” as in “C-10 PSMGFR”, “C-15 PSMGFR”, or “C-20 PSMGFR” refers to the number of amino acid residues that have been deleted at the C-terminal end of PSMGFR.

As used herein, the “extracellular domain of MUC1*” refers to the extracellular portion of a MUC1 protein that is devoid of the tandem repeat domain. In most cases, MUC1* is a cleavage product wherein the MUC1* portion consists of a short extracellular domain devoid of tandem repeats, a transmembrane domain and a cytoplasmic tail. The precise location of cleavage of MUC1 is not known perhaps because it appears that it can be cleaved by more than one enzyme. The extracellular domain of MUC1* will include most of the PSMGFR sequence but may have an additional 10-20 N-terminal amino acids.

As used herein, “NME family proteins” or “NME family member proteins”, numbered 1-10, are proteins grouped together because they all have at least one NDPK (nucleotide diphosphate kinase) domain. In some cases, the NDPK domain is not functional in terms of being able to catalyze the conversion of ATP to ADP. NME proteins were formally known as NM23 proteins, numbered H1, H2 and so on. Herein, the terms NM23 and NME are interchangeable. Herein, terms NME1, NME2, NME6 and NME7 are used to refer to the native protein as well as NME variants. In some cases these variants are more soluble, express better in E. coli or are more soluble than the native sequence protein. For example, NME7 as used in the specification can mean the native protein or a variant, such as NME7-AB that has superior commercial applicability because variations allow high yield expression of the soluble, properly folded protein in E. coli. “NME1” as referred to herein is interchangeable with “NM23-H1”. It is also intended that the invention not be limited by the exact sequence of the NME proteins. The mutant NME1-S120G, also called NM23-S120G, are used interchangeably throughout the application. The S120G mutants and the P96S mutant are preferred because of their preference for dimer formation, but may be referred to herein as NM23 dimers or NME1 dimers.

Various artificially created NME1 dimers are disclosed in PCT/US2012/036975, filed May 8, 2012, titled “Genetically Engineered Growth Factor Variants”, the contents of which are incorporated by reference herein as regards the dimeric growth factors that are disclosed therein.

NME7 as referred to herein is intended to mean native NME7 having a molecular weight of about 42 kDa, a cleaved form having a molecular weight between 25 and 33 kDa, a variant devoid of the DM10 leader sequence, NME7-AB or a recombinant NME7 protein, or variants thereof whose sequence may be altered to allow for efficient expression or that increase yield, solubility or other characteristics that make the NME7 more effective or commercially more viable.

As used herein, “NME1 dimers” also known as “NM23-H1 dimers” can be two NME1 proteins that are non-covalently bound to each other, two NME1 proteins covalently linked to each other or two NME1 proteins that are genetically fused together, including via a linker. NME1 dimers can be genetically engineered by making a DNA construct comprised of two NME1 proteins, which can be separated by a flexible linker. The two NME proteins need not be the full protein or the native sequence. For example, C-terminal deletions that promote dimer formation and stability may be used. Mutations such as S120G and/or P96S that promote dimer formation and stability may be used. Other NME family member dimers, such as NME2 or NME6 dimers, are similarly two NME proteins that are non-covalently bound to each other, two NME proteins covalently linked to each other or two NME proteins that are genetically fused together, including via a linker.

As used herein, an “an agent that maintains stem cells in the naïve state or reverts primed stem cells to the naïve state” refers to a protein, small molecule or nucleic acid that alone or in combination maintains stem cells in the naïve state, resembling cells of the inner cell mass of an embryo. Examples include but are not limited to NME1 dimers, human or bacterial, NME7, NME7-AB, 2i, 5i, nucleic acids such as siRNA that suppress expression of MBD3, CHD4, BRD4, or JMJD6.

As used herein, in reference to an agent being referred to as a “small molecule”, it may be a synthetic chemical or chemically based molecule having a molecular weight between 50 Da and 2000 Da, more preferably between 150 Da and 1000 Da, still more preferably between 200 Da and 750 Da.

As used herein, in reference to an agent being referred to as a “natural product”, it may be chemical molecule or a biological molecule, so long as the molecule exists in nature.

As used herein, “2i inhibitor” refers to small molecule inhibitors of GSK3-beta and MEK of the MAP kinase signaling pathway. The name 2i was coined in a research article (Silva J et al 2008), however herein “2i” refers to any inhibitor of either GSK3-beta or MEK, as there are many small molecules or biological agents that if they inhibit these targets, have the same effect on pluripotency or tumorigenesis.

As used herein, FGF, FGF-2 or bFGF refer to fibroblast growth factor.

As used herein, “Rho associated kinase inhibitors” may be small molecules, peptides or proteins (Rath N, et al, 2012). Rho kinase inhibitors are abbreviated here and elsewhere as ROCi or ROCKi, or Ri. The use of specific rho kinase inhibitors are meant to be exemplary and can be substituted for any other rho kinase inhibitor.

As used herein, the term “stem-like” refers to a state in which cells acquire characteristics of stem cells or progenitor cells, share important elements of the gene expression profile of stem cells progenitor cells. Stem-like cells may be somatic cells undergoing induction to a less mature state, such as increasing expression of pluripotency genes. Stem-like cells also refers to cells that have undergone some de-differentiation or are in a meta-stable state from which they can alter their terminal differentiation.

Sequence Listing Free Text

As regards the use of nucleotide symbols other than a, g, c, t, they follow the convention set forth in WIPO Standard ST.25, Appendix 2, Table 1, wherein k represents t or g; n represents a, c, t or g; m represents a or c; r represents a or g; s represents c or g; w represents a or t and y represents c or t.

(SEQ ID NO: 1)

MTPGTQSPFF LLLLLTVLTV VTGSGHASST PGGEKETSAT
QRSSVPSSTE KNAVSMTSSV LSSHSPGSGS STTQGQDVTL
APATEPASGS AATWGQDVTS VPVTRPALGS TTPPAHDVTS
APDNKPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDTRPAPGS TAPPAHGVTS
APDTRPAPGS TAPPAHGVTS APDNRPALGS TAPPVHNVTS
ASGSASGSAS TLVHNGTSAR ATTTPASKST PFSIPSHHSD
TPTTLASHST KTDASSTHHS SVPPLTSSNH STSPQLSTGV
SFFFLSFHIS NLQFNSSLED PSTDYYQELQ RDISEMFLQI
YKQGGFLGLS NIKFRPGSVV VQLTLAFREG TINVHDVETQ
FNQYKTEAAS RYNLTISDVS VSDVPFPFSA QSGAGVPGWG
IALLVLVCVL VALAIVYLIA LAVCQCRRKN YGQLDIFPAR
DTYHPMSEYP TYHTHGRYVP PSSTDRSPYE KVSAGNGGSS
LSYTNPAVAA ASANL

describes full-length human MUC1 Receptor (Mucin 1 precursor, Genbank Accession number: P15941).

(SEQ ID NO: 2)

MTPGTQSPFFLLLLLTVLT

(SEQ ID NO: 3)

MTPGTQSPFFLLLLLTVLT VVTA

(SEQ ID NO: 4)

MTPGTQSPFFLLLLLTVLT VVTG

SEQ ID NOS:2, 3 and 4 describe N-terminal MUC-1 signaling sequence for directing MUC1 receptor and truncated isoforms to cell membrane surface. Up to 3 amino acid residues may be absent at C-terminal end as indicated by variants in SEQ ID NOS:2, 3 and 4.

GTINVHDVETQFNQYKTEAASRYNLTISDVSVSDVPFPFSAQSGAGVPGW GIALLVLVCVLVALAIVYLIALAVCQCRRKNYGQLDIFPARDTYHPMSEYPTYHTHG RYVPPSSTDRSPYEKVSAGNGGSSLSYTNPAVAAASANL (SEQ ID NO:5) describes a truncated MUC1 receptor isoform having nat-PSMGFR at its N-terminus and including the transmembrane and cytoplasmic sequences of a human full-length MUC1 receptor.

GTINVHDVETQFNQYKTEAASRYNLTISDVSVSDVPFPFSAQSGA (SEQ ID NO:6) describes the extracellular domain of Native Primary Sequence of the human MUC1 Growth Factor Receptor (nat-PSMGFR—an example of “PSMGFR”):

TINVHDVETQFNQYKTEAASRYNLTISDVSVSDVPFPFSAQSGA (SEQ ID NO:7) describes the extracellular domain of Native Primary Sequence of the human MUC1 Growth Factor Receptor (nat-PSMGFR—An example of “PSMGFR”), having a single amino acid deletion at the N-terminus of SEQ ID NO:6).

GTINVHDVETQFNQYKTEAASPYNLTISDVSVSDVPFPFSAQSGA (SEQ ID NO: 8) describes the extracellular domain of “SPY” functional variant of the native Primary Sequence of the MUC1 Growth Factor Receptor having enhanced stability (var-PSMGFR—An example of “PSMGFR”).

TINVHDVETQFNQYKTEAASPYNLTISDVSVSDVPFPFSAQSGA (SEQ ID NO:9) describes the extracellular domain of “SPY” functional variant of the native Primary Sequence of the MUC1 Growth Factor Receptor having enhanced stability (var-PSMGFR—An example of “PSMGFR”), having a single amino acid deletion at the C-terminus of SEQ ID NO:8).

tgtcagtgccgccgaaagaactacgggcagctggacatctttccagcccgggatacctaccatcctatgagcgagta ccccacctaccacacccatgggcgctatgtgccccctagcagtaccgatcgtagcccctatgagaaggtttctgcaggtaacggtggc agcagcctctcttacacaaacccagcagtggcagccgcttctgccaacttg (SEQ ID NO:10) describes human MUC1 cytoplasmic domain nucleotide sequence.

CQCRRKNYGQLDIFPARDTYHPMSEYPTYHTHGRYVPPSSTDRSPYEKVS AGNGGSSLSYTNPAVAAASANL (SEQ ID NO:11) describes human MUC1 cytoplasmic domain amino acid sequence.

gagatcctgagacaatgaatcatagtgaaagattcgttttcattgcagagtggtatgatccaaatgcttcacttcttcgac gttatgagcttttattttacccaggggatggatctgttgaaatgcatgatgtaaagaatcatcgcacctttttaaagcggaccaaatatgata acctgcacttggaagatttatttataggcaacaaagtgaatgtcttttctcgacaactggtattaattgactatggggatcaatatacagctc gccagctgggcagtaggaaagaaaaaacgctagccctaattaaaccagatgcaatatcaaaggctggagaaataattgaaataataa acaaagctggatttactataaccaaactcaaaatgatgatgctttcaaggaaagaagcattggattttcatgtagatcaccagtcaagacc ctttttcaatgagctgatccagtttattacaactggtcctattattgccatggagattttaagagatgatgctatatgtgaatggaaaagactg ctgggacctgcaaactctggagtggcacgcacagatgcttctgaaagcattagagccctctttggaacagatggcataagaaatgcag cgcatggccctgattcttttgcttctgcggccagagaaatggagttgttttttccttcaagtggaggttgtgggccggcaaacactgctaa atttactaattgtacctgttgcattgttaaaccccatgctgtcagtgaaggtatgttgaatacactatattcagtacattttgttaataggagag caatgtttattttcttgatgtactttatgtatagaaaataa (SEQ ID NO:12) describes human NME7 nucleotide sequence (NME7: GENBANK ACCESSION AB209049).

DPETMNHSERFVFIAEWYDPNASLLRRYELLFYPGDGSVEMHDVKNHRT FLKRTKYDNLHLEDLFIGNKVNVFSRQLVLIDYGDQYTARQLGSRKEKTLALIKPDAI SKAGEIIEIINKAGFTITKLKMMMLSRKEALDFHVDHQSRPFFNELIQFITTGPIIAMEIL RDDAICEWKRLLGPANSGVARTDASESIRALFGTDGIRNAAHGPDSFASAAREMELF FPSSGGCGPANTAKFTNCTCCIVKPHAVSEGMLNTLYSVHFVNRRAMFIFLMYFMY RK (SEQ ID NO:13) describes human NME7 amino acid sequence (NME7: GENBANK ACCESSION AB209049).

atggtgctactgtctactttagggatcgtctttcaaggcgaggggcctcctatctcaagctgtgatacaggaaccatgg ccaactgtgagcgtaccttcattgcgatcaaaccagatggggtccagcggggtcttgtgggagagattatcaagcgttttgagcagaaa ggattccgccttgttggtctgaaattcatgcaagcttccgaagatcttctcaaggaacactacgttgacctgaaggaccgtccattctttgc cggcctggtgaaatacatgcactcagggccggtagttgccatggtctgggaggggctgaatgtggtgaagacgggccgagtcatgct cggggagaccaaccctgcagactccaagcctgggaccatccgtggagacttctgcatacaagttggcaggaacattatacatggcag tgattctgtggagagtgcagagaaggagatcggcttgtggtttcaccctgaggaactggtagattacacgagctgtgctcagaactgga tctatgaatga (SEQ ID NO:14) describes human NM23-H1 also known as NME1 nucleotide sequence (NM23-H1: GENBANK ACCESSION AF487339).

MVLLSTLGIVFQGEGPPISSCDTGTMANCERTFIAIKPDGVQRGLVGEIIKR FEQKGFRLVGLKFMQASEDLLKEHYVDLKDRPFFAGLVKYMHSGPVVAMVWEGL NVVKTGRVMLGETNPADSKPGTIRGDFCIQVGRNIIHGSDSVESAEKEIGLWFHPEEL VDYTSCAQNWIYE (SEQ ID NO:15) NM23-H1 describes amino acid sequence (NM23-H1 also known as NME1: GENBANK ACCESSION AF487339).

atggtgctactgtctactttagggatcgtctttcaaggcgaggggcctcctatctcaagctgtgatacaggaaccatgg ccaactgtgagcgtaccttcattgcgatcaaaccagatggggtccagcggggtcttgtgggagagattatcaagcgttttgagcagaaa ggattccgccttgttggtctgaaattcatgcaagchccgaagatcttctcaaggaacactacgttgacctgaaggaccgtccattctttgc cggcctggtgaaatacatgcactcagggccggtagttgccatggtctgggaggggctgaatgtggtgaagacgggccgagtcatgct cggggagaccaaccctgcagactccaagcctgggaccatccgtggagacttctgcatacaagttggcaggaacattatacatggcgg tgattctgtggagagtgcagagaaggagatcggcttgtggtttcaccctgaggaactggtagattacacgagctgtgctcagaactgga tctatgaatga (SEQ ID NO:16) describes human NM23-H1 S120G mutant nucleotide sequence (NM23-H1: GENBANK ACCESSION AF487339).

MVLLSTLGIVFQGEGPPISSCDTGTMANCERTFIAIKPDGVQRGLVGEIIKR FEQKGFRLVGLKFMQASEDLLKEHYVDLKDRPFFAGLVKYMHSGPVVAMVWEGL NVVKTGRVMLGETNPADSKPGTIRGDFCIQVGRNIIHGGDSVESAEKEIGLWFHPEEL VDYTSCAQNWIYE (SEQ ID NO:17) describes NM23-H1 S120G mutant amino acid sequence (NM23-H1: GENBANK ACCESSION AF487339).

atggccaacctggagcgcaccttcatcgccatcaagccggacggcgtgcagcgcggcctggtgggcgagatcatc aagcgcttcgagcagaagggattccgcctcgtggccatgaagttcctccgggcctctgaagaacacctgaagcagcactacattgac ctgaaagaccgaccattcttccctgggctggtgaagtacatgaactcagggccggttgtggccatggtctgggaggggctgaacgtg gtgaagacaggccgagtgatgcttggggagaccaatccagcagattcaaagccaggcaccattcgtggggacttctgcattcaggtt ggcaggaacatcattcatggcagtgattcagtaaaaagtgctgaaaaagaaatcagcctatggtttaagcctgaagaactggttgacta caagtcttgtgctcatgactgggtctatgaataa (SEQ ID NO:18) describes human NM23-H2 nucleotide sequence (NM23-H2: GENBANK ACCESSION AK313448).

MANLERTFIAIKPDGVQRGLVGEIIKRFEQKGFRLVAMKFLRASEEHLKQ HYIDLKDRPFFPGLVKYMNSGPVVAMVWEGLNVVKTGRVMLGETNPADSKPGTIR GDFCIQVGRNIIHGSDSVKSAEKEISLWFKPEELVDYKSCAHDWVYE (SEQ ID NO:19) describes NM23-H2 amino acid sequence (NM23-H2: GENBANK ACCESSION AK313448).

Human NM23-H7-2 isoform b sequence optimized for E. coli expression:

(DNA)

(SEQ ID NO: 20)

atgcatgacgttaaaaatcaccgtacctttctgaaacgcacgaaatatga
taatctgcatctggaagacctgtttattggcaacaaagtcaatgtgttct
ctcgtcagctggtgctgatcgattatggcgaccagtacaccgcgcgtcaa
ctgggtagtcgcaaagaaaaaacgctggccctgattaaaccggatgcaat
ctccaaagctggcgaaattatcgaaattatcaacaaagcgggtttcacca
tcacgaaactgaaaatgatgatgctgagccgtaaagaagccctggatttt
catgtcgaccaccagtctcgcccgtttttcaatgaactgattcaattcat
caccacgggtccgattatcgcaatggaaattctgcgtgatgacgctatct
gcgaatggaaacgcctgctgggcccggcaaactcaggtgttgcgcgtacc
gatgccagtgaatccattcgcgctctgtttggcaccgatggtatccgtaa
tgcagcacatggtccggactcattcgcatcggcagctcgtgaaatggaac
tgtttttcccgagctctggcggttgcggtccggcaaacaccgccaaattt
accaattgtacgtgctgtattgtcaaaccgcacgcagtgtcagaaggcct
gctgggtaaaattctgatggcaatccgtgatgctggctttgaaatctcgg
ccatgcagatgttcaacatggaccgcgttaacgtcgaagaattctacgaa
gtttacaaaggcgtggttaccgaatatcacgatatggttacggaaatgta
ctccggtccgtgcgtcgcgatggaaattcagcaaaacaatgccaccaaaa
cgtttcgtgaattctgtggtccggcagatccggaaatcgcacgtcatctg
cgtccgggtaccctgcgcgcaatttttggtaaaacgaaaatccagaacgc
tgtgcactgtaccgatctgccggaagacggtctgctggaagttcaatact
ttttcaaaattctggataattga
(amino acids)

(SEQ ID NO: 21)

MHDVKNHRTFLKRTKYDNLHLEDLFIGNKVNVFSRQLVLIDYGDQYTARQ
LGSRKEKTLALIKPDAISKAGEIIEIINKAGFTITKLKMMMLSRKEALDF
HVDHQSRPFFNELIQFITTGPIIAMEILRDDAICEWKRLLGPANSGVART
DASESIRALFGTDGIRNAAHGPDSFASAAREMELFFPSSGGCGPANTAKF
TNCTCCIVKPHAVSEGLLGKILMAIRDAGFEISAMQMFNMDRVNVEEFYE
VYKGVVTEYHDMVTEMYSGPCVAMEIQQNNATKTFREFCGPADPEIARHL
RPGTLRAIFGKTKIQNAVHCTDLPEDGLLEVQYFFKILDN-

Human NME7-A:

(DNA)

(SEQ ID NO: 22)

atggaaaaaacgctagccctaattaaaccagatgcaatatcaaaggctgg
agaaataattgaaataataaacaaagctggatttactataaccaaactca
aaatgatgatgctttcaaggaaagaagcattggattttcatgtagatcac
cagtcaagaccctttttcaatgagctgatccagtttattacaactggtcc
tattattgccatggagattttaagagatgatgctatatgtgaatggaaaa
gactgctgggacctgcaaactctggagtggcacgcacagatgcttctgaa
agcattagagccctctttggaacagatggcataagaaatgcagcgcatgg
ccctgattcttttgcttctgcggccagagaaatggagttgtttttttga
(amino acids)

(SEQ ID NO: 23)

MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFF-

Human NME7-A1:

(DNA)

(SEQ ID NO: 24)

atggaaaaaacgctagccctaattaaaccagatgcaatatcaaaggctgg
agaaataattgaaataataaacaaagctggatttactataaccaaactca
aaatgatgatgctttcaaggaaagaagcattggattttcatgtagatcac
cagtcaagaccctttttcaatgagctgatccagtttattacaactggtcc
tattattgccatggagattttaagagatgatgctatatgtgaatggaaaa
gactgctgggacctgcaaactctggagtggcacgcacagatgcttctgaa
agcattagagccctctttggaacagatggcataagaaatgcagcgcatgg
ccctgattcttttgcttctgcggccagagaaatggagttgttttttcctt
caagtggaggttgtgggccggcaaacactgctaaatttacttga
(amino acids)

(SEQ ID NO: 25)

MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFFPSSGGCGPANTAKFT-

Human NME7-A2:

(DNA)

(SEQ ID NO: 26)

atgaatcatagtgaaagattcgttttcattgcagagtggtatgatccaaa
tgcttcacttcttcgacgttatgagcttttattttacccaggggatggat
ctgttgaaatgcatgatgtaaagaatcatcgcacctttttaaagcggacc
aaatatgataacctgcacttggaagatttatttataggcaacaaagtgaa
tgtcttttctcgacaactggtattaattgactatggggatcaatatacag
ctcgccagctgggcagtaggaaagaaaaaacgctagccctaattaaacca
gatgcaatatcaaaggctggagaaataattgaaataataaacaaagctgg
atttactataaccaaactcaaaatgatgatgctttcaaggaaagaagcat
tggattttcatgtagatcaccagtcaagaccctttttcaatgagctgatc
cagtttattacaactggtcctattattgccatggagattttaagagatga
tgctatatgtgaatggaaaagactgctgggacctgcaaactctggagtgg
cacgcacagatgcttctgaaagcattagagccctctttggaacagatggc
ataagaaatgcagcgcatggccctgattcttttgcttctgcggccagaga
aatggagttgtttttttga
(amino acids)

(SEQ ID NO: 27)

MNHSERFVFIAEWYDPNASLLRRYELLFYPGDGSVEMHDVKNHRTFLKRT
KYDNLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITTGPIIAMEILRDDAICEWKRLLGPANSGVARTDASESIRALFGTDG
IRNAAHGPDSFASAAREMELFF-

Human NME7-A3:

(DNA)

(SEQ ID NO: 28)

atgaatcatagtgaaagattcgttttcattgcagagtggtatgatccaaa
tgcttcacttcttcgacgttatgagcttttattttacccaggggatggat
ctgttgaaatgcatgatgtaaagaatcatcgcacctttttaaagcggacc
aaatatgataacctgcacttggaagatttatttataggcaacaaagtgaa
tgtcttttctcgacaactggtattaattgactatggggatcaatatacag
ctcgccagctgggcagtaggaaagaaaaaacgctagccctaattaaacca
gatgcaatatcaaaggctggagaaataattgaaataataaacaaagctgg
atttactataaccaaactcaaaatgatgatgctttcaaggaaagaagcat
tggattttcatgtagatcaccagtcaagaccctttttcaatgagctgatc
cagtttattacaactggtcctattattgccatggagattttaagagatga
tgctatatgtgaatggaaaagactgctgggacctgcaaactctggagtgg
cacgcacagatgcttctgaaagcattagagccctctttggaacagatggc
ataagaaatgcagcgcatggccctgattcttttgcttctgcggccagaga
aatggagttgttttttccttcaagtggaggttgtgggccggcaaacactg
ctaaatttacttga
(amino acids)

(SEQ ID NO: 29)

MNHSERFVFIAEWYDPNASLLRRYELLFYPGDGSVEMHDVKNHRTFLKRT
KYDNLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITTGPIIAMEILRDDAICEWKRLLGPANSGVARTDASESIRALFGTDG
IRNAAHGPDSFASAAREMELFFPSSGGCGPANTAKFT-

Human NME7-B:

(DNA)

(SEQ ID NO: 30)

atgaattgtacctgttgcattgttaaaccccatgctgtcagtgaaggact
gttgggaaagatcctgatggctatccgagatgcaggttttgaaatctcag
ctatgcagatgttcaatatggatcgggttaatgttgaggaattctatgaa
gtttataaaggagtagtgaccgaatatcatgacatggtgacagaaatgta
ttctggcccttgtgtagcaatggagattcaacagaataatgctacaaaga
catttcgagaattttgtggacctgctgatcctgaaattgcccggcattta
cgccctggaactctcagagcaatctttggtaaaactaagatccagaatgc
tgttcactgtactgatctgccagaggatggcctattagaggttcaatact
tcttctga
(amino acids)

(SEQ ID NO: 31)

MNCTCCIVKPHAVSEGLLGKILMAIRDAGFEISAMQMFNMDRVNVEEFYE
VYKGVVTEYHDMVTEMYSGPCVAMEIQQNNATKTFREFCGPADPEIARHL
RPGTLRAIFGKTKIQNAVHCTDLPEDGLLEVQYFF-

Human NME7-B1:

(DNA)

(SEQ ID NO: 32)

atgaattgtacctgttgcattgttaaaccccatgctgtcagtgaaggact
gttgggaaagatcctgatggctatccgagatgcaggttttgaaatctcag
ctatgcagatgttcaatatggatcgggttaatgttgaggaattctatgaa
gtttataaaggagtagtgaccgaatatcatgacatggtgacagaaatgta
ttctggcccttgtgtagcaatggagattcaacagaataatgctacaaaga
catttcgagaattttgtggacctgctgatcctgaaattgcccggcattta
cgccctggaactctcagagcaatctttggtaaaactaagatccagaatgc
tgttcactgtactgatctgccagaggatggcctattagaggttcaatact
tcttcaagatcttggataattagtga
(amino acids)

(SEQ ID NO: 33)

MNCTCCIVKPHAVSEGLLGKILMAIRDAGFEISAMQMFNMDRVNVEEFYE
VYKGVVTEYHDMVTEMYSGPCVAMEIQQNNATKTFREFCGPADPEIARHL
RPGTLRAIFGKTKIQNAVHCTDLPEDGLLEVQYFFKILDN-

Human NME7-B2:

(DNA)

(SEQ ID NO: 34)

atgccttcaagtggaggttgtgggccggcaaacactgctaaatttactaa
ttgtacctgttgcattgttaaaccccatgctgtcagtgaaggactgttgg
gaaagatcctgatggctatccgagatgcaggttttgaaatctcagctatg
cagatgttcaatatggatcgggttaatgttgaggaattctatgaagttta
taaaggagtagtgaccgaatatcatgacatggtgacagaaatgtattctg
gcccttgtgtagcaatggagattcaacagaataatgctacaaagacattt
cgagaattttgtggacctgctgatcctgaaattgcccggcatttacgccc
tggaactctcagagcaatctttggtaaaactaagatccagaatgctgttc
actgtactgatctgccagaggatggcctattagaggttcaatacttcttc
tga
(amino acids)

(SEQ ID NO: 35)

MPSSGGCGPANTAKFTNCTCCIVKPHAVSEGLLGKILMAIRDAGFEISAM
QMFNMDRVNVEEFYEVYKGVVTEYHDMVTEMYSGPCVAMEIQQNNATKTF
REFCGPADPEIARHLRPGTLRAIFGKTKIQNAVHCTDLPEDGLLEVQYFF
-

Human NME7-B3:

(DNA)

(SEQ ID NO: 36)

atgccttcaagtggaggttgtgggccggcaaacactgctaaatttactaa
ttgtacctgttgcattgttaaaccccatgctgtcagtgaaggactgttgg
gaaagatcctgatggctatccgagatgcaggttttgaaatctcagctatg
cagatgttcaatatggatcgggttaatgttgaggaattctatgaagttta
taaaggagtagtgaccgaatatcatgacatggtgacagaaatgtattctg
gcccttgtgtagcaatggagattcaacagaataatgctacaaagacattt
cgagaattttgtggacctgctgatcctgaaattgcccggcatttacgccc
tggaactctcagagcaatctttggtaaaactaagatccagaatgctgttc
actgtactgatctgccagaggatggcctattagaggttcaatacttcttc
aagatcttggataattagtga
(amino acids)

(SEQ ID NO: 37)

MPSSGGCGPANTAKFTNCTCCIVKPHAVSEGLLGKILMAIRDAGFEISAM
QMFNMDRVNVEEFYEVYKGVVTEYHDMVTEMYSGPCVAMEIQQNNATKTF
REFCGPADPEIARHLRPGTLRAIFGKTKIQNAVHCTDLPEDGLLEVQYFF
KILDN--

Human NME7-AB:

(DNA)

(SEQ ID NO: 38)

atggaaaaaacgctagccctaattaaaccagatgcaatatcaaaggctgg
agaaataattgaaataataaacaaagctggatttactataaccaaactca
aaatgatgatgctttcaaggaaagaagcattggattttcatgtagatcac
cagtcaagaccctttttcaatgagctgatccagtttattacaactggtcc
tattattgccatggagattttaagagatgatgctatatgtgaatggaaaa
gactgctgggacctgcaaactctggagtggcacgcacagatgcttctgaa
agcattagagccctctttggaacagatggcataagaaatgcagcgcatgg
ccctgattcttttgcttctgcggccagagaaatggagttgttttttcctt
caagtggaggttgtgggccggcaaacactgctaaatttactaattgtacc
tgttgcattgttaaaccccatgctgtcagtgaaggactgttgggaaagat
cctgatggctatccgagatgcaggttttgaaatctcagctatgcagatgt
tcaatatggatcgggttaatgttgaggaattctatgaagtttataaagga
gtagtgaccgaatatcatgacatggtgacagaaatgtattctggcccttg
tgtagcaatggagattcaacagaataatgctacaaagacatttcgagaat
tttgtggacctgctgatcctgaaattgcccggcatttacgccctggaact
ctcagagcaatctttggtaaaactaagatccagaatgctgttcactgtac
tgatctgccagaggatggcctattagaggttcaatacttcttcaagatct
tggataattagtga
(amino acids)

(SEQ ID NO: 39)

MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFFPSSGGCGPANTAKFTNCT
CCIVKPHAVSEGLLGKILMAIRDAGFEISAMQMFNMDRVNVEEFYEVYKG
VVTEYHDMVTEMYSGPCVAMEIQQNNATKTFREFCGPADPEIARHLRPGT
LRAIFGKTKIQNAVHCTDLPEDGLLEVQYFFKILDN--

Human NME7-AB1:

(DNA)

(SEQ ID NO: 40)

atggaaaaaacgctagccctaattaaaccagatgcaatatcaaaggctgg
agaaataattgaaataataaacaaagctggatttactataaccaaactca
aaatgatgatgctttcaaggaaagaagcattggattttcatgtagatcac
cagtcaagaccctttttcaatgagctgatccagtttattacaactggtcc
tattattgccatggagattttaagagatgatgctatatgtgaatggaaaa
gactgctgggacctgcaaactctggagtggcacgcacagatgcttctgaa
agcattagagccctctttggaacagatggcataagaaatgcagcgcatgg
ccctgattcttttgcttctgcggccagagaaatggagttgttttttcctt
caagtggaggttgtgggccggcaaacactgctaaatttactaattgtacc
tgttgcattgttaaaccccatgctgtcagtgaaggactgttgggaaagat
cctgatggctatccgagatgcaggttttgaaatctcagctatgcagatgt
tcaatatggatcgggttaatgttgaggaattctatgaagtttataaagga
gtagtgaccgaatatcatgacatggtgacagaaatgtattctggcccttg
tgtagcaatggagattcaacagaataatgctacaaagacatttcgagaat
tttgtggacctgctgatcctgaaattgcccggcatttacgccctggaact
ctcagagcaatctttggtaaaactaagatccagaatgctgttcactgtac
tgatctgccagaggatggcctattagaggttcaatacttcttctga
(amino acids)

(SEQ ID NO: 41)

MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFFPSSGGCGPANTAKFTNCT
CCIVKPHAVSEGLLGKILMAIRDAGFEISAMQMFNMDRVNVEEFYEVYKG
VVTEYHDMVTEMYSGPCVAMEIQQNNATKTFREFCGPADPEIARHLRPGT
LRAIFGKTKIQNAVHCTDLPEDGLLEVQYFF-

Human NME7-A sequence optimized for E. coli expression:

(DNA)

(SEQ ID NO: 42)

atggaaaaaacgctggccctgattaaaccggatgcaatctccaaagctgg
cgaaattatcgaaattatcaacaaagcgggtttcaccatcacgaaactga
aaatgatgatgctgagccgtaaagaagccctggattttcatgtcgaccac
cagtctcgcccgtttttcaatgaactgattcaattcatcaccacgggtcc
gattatcgcaatggaaattctgcgtgatgacgctatctgcgaatggaaac
gcctgctgggcccggcaaactcaggtgttgcgcgtaccgatgccagtgaa
tccattcgcgctctgtttggcaccgatggtatccgtaatgcagcacatgg
tccggactcattcgcatcggcagctcgtgaaatggaactgtttttctga
(amino acids)

(SEQ ID NO: 43)

MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFF-

Human NME7-A1 sequence optimized for E. coli expression:

(DNA)

(SEQ ID NO: 44)

atggaaaaaacgctggccctgattaaaccggatgcaatctccaaagctgg
cgaaattatcgaaattatcaacaaagcgggtttcaccatcacgaaactga
aaatgatgatgctgagccgtaaagaagccctggattttcatgtcgaccac
cagtctcgcccgtttttcaatgaactgattcaattcatcaccacgggtcc
gattatcgcaatggaaattctgcgtgatgacgctatctgcgaatggaaac
gcctgctgggcccggcaaactcaggtgttgcgcgtaccgatgccagtgaa
tccattcgcgctctgtttggcaccgatggtatccgtaatgcagcacatgg
tccggactcattcgcatcggcagctcgtgaaatggaactgtttttcccga
gctctggcggttgcggtccggcaaacaccgccaaatttacctga
(amino acids)

(SEQ ID NO: 45)

MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFFPSSGGCGPANTAKFT-

Human NME7-A2 sequence optimized for E. coli expression:

(DNA)

(SEQ ID NO: 46)

atgaatcactccgaacgctttgtttttatcgccgaatggtatgacccgaa
tgcttccctgctgcgccgctacgaactgctgttttatccgggcgatggta
gcgtggaaatgcatgacgttaaaaatcaccgtacctttctgaaacgcacg
aaatatgataatctgcatctggaagacctgtttattggcaacaaagtcaa
tgtgttctctcgtcagctggtgctgatcgattatggcgaccagtacaccg
cgcgtcaactgggtagtcgcaaagaaaaaacgctggccctgattaaaccg
gatgcaatctccaaagctggcgaaattatcgaaattatcaacaaagcggg
tttcaccatcacgaaactgaaaatgatgatgctgagccgtaaagaagccc
tggattttcatgtcgaccaccagtctcgcccgtttttcaatgaactgatt
caattcatcaccacgggtccgattatcgcaatggaaattctgcgtgatga
cgctatctgcgaatggaaacgcctgctgggcccggcaaactcaggtgttg
cgcgtaccgatgccagtgaatccattcgcgctctgtttggcaccgatggt
atccgtaatgcagcacatggtccggactcattcgcatcggcagctcgtga
aatggaactgtttttctga
(amino acids)

(SEQ ID NO: 47)

MNHSERFVFIAEWYDPNASLLRRYELLFYPGDGSVEMHDVKNHRTFLKRT
KYDNLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITTGPIIAMEILRDDAICEWKRLLGPANSGVARTDASESIRALFGTDG
IRNAAHGPDSFASAAREMELFF-

Human NME7-A3 sequence optimized for E. coli expression:

(DNA)

(SEQ ID NO: 48)

atgaatcactccgaacgctttgtttttatcgccgaatggtatgacccgaa
tgcttccctgctgcgccgctacgaactgctgttttatccgggcgatggta
gcgtggaaatgcatgacgttaaaaatcaccgtacctttctgaaacgcacg
aaatatgataatctgcatctggaagacctgtttattggcaacaaagtcaa
tgtgttctctcgtcagctggtgctgatcgattatggcgaccagtacaccg
cgcgtcaactgggtagtcgcaaagaaaaaacgctggccctgattaaaccg
gatgcaatctccaaagctggcgaaattatcgaaattatcaacaaagcggg
tttcaccatcacgaaactgaaaatgatgatgctgagccgtaaagaagccc
tggattttcatgtcgaccaccagtctcgcccgtttttcaatgaactgatt
caattcatcaccacgggtccgattatcgcaatggaaattctgcgtgatga
cgctatctgcgaatggaaacgcctgctgggcccggcaaactcaggtgttg
cgcgtaccgatgccagtgaatccattcgcgctctgtttggcaccgatggt
atccgtaatgcagcacatggtccggactcattcgcatcggcagctcgtga
aatggaactgtttttcccgagctctggcggttgcggtccggcaaacaccg
ccaaatttacctga
(amino acids)

(SEQ ID NO: 49)

MNHSERFVFIAEWYDPNASLLRRYELLFYPGDGSVEMHDVKNHRTFLKRT
KYDNLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITTGPIIAMEILRDDAICEWKRLLGPANSGVARTDASESIRALFGTDG
IRNAAHGPDSFASAAREMELFFPSSGGCGPANTAKFT-

Human NME7-B sequence optimized for E. coli expression:

(DNA)

(SEQ ID NO: 50)

atgaattgtacgtgctgtattgtcaaaccgcacgcagtgtcagaaggcct
gctgggtaaaattctgatggcaatccgtgatgctggctttgaaatctcgg
ccatgcagatgttcaacatggaccgcgttaacgtcgaagaattctacgaa
gtttacaaaggcgtggttaccgaatatcacgatatggttacggaaatgta
ctccggtccgtgcgtcgcgatggaaattcagcaaaacaatgccaccaaaa
cgtttcgtgaattctgtggtccggcagatccggaaatcgcacgtcatctg
cgtccgggtaccctgcgcgcaatttttggtaaaacgaaaatccagaacgc
tgtgcactgtaccgatctgccggaagacggtctgctggaagttcaatact
ttttctga
(amino acids)

(SEQ ID NO: 51)

MNCTCCIVKPHAVSEGLLGKILMAIRDAGFEISAMQMFNMDRVNVEEFYE
VYKGVVTEYHDMVTEMYSGPCVAMEIQQNNATKTFREFCGPADPEIARHL
RPGTLRAIFGKTKIQNAVHCTDLPEDGLLEVQYFF-

Human NME7-B1 sequence optimized for E. coli expression:

(DNA)

(SEQ ID NO: 52)

atgaattgtacgtgctgtattgtcaaaccgcacgcagtgtcagaaggcct
gctgggtaaaattctgatggcaatccgtgatgctggctttgaaatctcgg
ccatgcagatgttcaacatggaccgcgttaacgtcgaagaattctacgaa
gtttacaaaggcgtggttaccgaatatcacgatatggttacggaaatgta
ctccggtccgtgcgtcgcgatggaaattcagcaaaacaatgccaccaaaa
cgtttcgtgaattctgtggtccggcagatccggaaatcgcacgtcatctg
cgtccgggtaccctgcgcgcaatttttggtaaaacgaaaatccagaacgc
tgtgcactgtaccgatctgccggaagacggtctgctggaagttcaatact
ttttcaaaattctggataattga
(amino acids)

(SEQ ID NO: 53)

MNCTCCIVKPHAVSEGLLGKILMAIRDAGFEISAMQMFNMDRVNVEEFYE
VYKGVVTEYHDMVTEMYSGPCVAMEIQQNNATKTFREFCGPADPEIARHL
RPGTLRAIFGKTKIQNAVHCTDLPEDGLLEVQYFFKILDN-

Human NME7-B2 sequence optimized for E. coli expression:

(DNA)

(SEQ ID NO: 54)

atgccgagctctggcggttgcggtccggcaaacaccgccaaatttaccaa
ttgtacgtgctgtattgtcaaaccgcacgcagtgtcagaaggcctgctgg
gtaaaattctgatggcaatccgtgatgctggctttgaaatctcggccatg
cagatgttcaacatggaccgcgttaacgtcgaagaattctacgaagttta
caaaggcgtggttaccgaatatcacgatatggttacggaaatgtactccg
gtccgtgcgtcgcgatggaaattcagcaaaacaatgccaccaaaacgttt
cgtgaattctgtggtccggcagatccggaaatcgcacgtcatctgcgtcc
gggtaccctgcgcgcaatttttggtaaaacgaaaatccagaacgctgtgc
actgtaccgatctgccggaagacggtctgctggaagttcaatactttttc
tga
(amino acids)

(SEQ ID NO: 55)

MPSSGGCGPANTAKFTNCTCCIVKPHAVSEGLLGKILMAIRDAGFEISAM
QMFNMDRVNVEEFYEVYKGVVTEYHDMVTEMYSGPCVAMEIQQNNATKTF
REFCGPADPEIARHLRPGTLRAIFGKTKIQNAVHCTDLPEDGLLEVQ
YFF-

Human NME7-B3 sequence optimized for E. coli expression:

(DNA)

(SEQ ID NO: 56)

atgccgagctctggcggttgcggtccggcaaacaccgccaaatttaccaa
ttgtacgtgctgtattgtcaaaccgcacgcagtgtcagaaggcctgctgg
gtaaaattctgatggcaatccgtgatgctggctttgaaatctcggccatg
cagatgttcaacatggaccgcgttaacgtcgaagaattctacgaagttta
caaaggcgtggttaccgaatatcacgatatggttacggaaatgtactccg
gtccgtgcgtcgcgatggaaattcagcaaaacaatgccaccaaaacgttt
cgtgaattctgtggtccggcagatccggaaatcgcacgtcatctgcgtcc
gggtaccctgcgcgcaatttttggtaaaacgaaaatccagaacgctgtgc
actgtaccgatctgccggaagacggtctgctggaagttcaatactttttc
aaaattctggataattga
(amino acids)

(SEQ ID NO: 57)

MPSSGGCGPANTAKFTNCTCCIVKPHAVSEGLLGKILMAIRDAGFEISAM
QMFNMDRVNVEEFYEVYKGVVTEYHDMVTEMYSGPCVAMEIQQNNATKTF
REFCGPADPEIARHLRPGTLRAIFGKTKIQNAVHCTDLPEDGLLEVQYFF
KILDN-

Human NME7-AB sequence optimized for E. coli expression:

(DNA)

(SEQ ID NO: 58)

atggaaaaaacgctggccctgattaaaccggatgcaatctccaaagctgg
cgaaattatcgaaattatcaacaaagcgggtttcaccatcacgaaactga
aaatgatgatgctgagccgtaaagaagccctggattttcatgtcgaccac
cagtctcgcccgtttttcaatgaactgattcaattcatcaccacgggtcc
gattatcgcaatggaaattctgcgtgatgacgctatctgcgaatggaaac
gcctgctgggcccggcaaactcaggtgttgcgcgtaccgatgccagtgaa
tccattcgcgctctgtttggcaccgatggtatccgtaatgcagcacatgg
tccggactcattcgcatcggcagctcgtgaaatggaactgtttttcccga
gctctggcggttgcggtccggcaaacaccgccaaatttaccaattgtacg
tgctgtattgtcaaaccgcacgcagtgtcagaaggcctgctgggtaaaat
tctgatggcaatccgtgatgctggctttgaaatctcggccatgcagatgt
tcaacatggaccgcgttaacgtcgaagaattctacgaagtttacaaaggc
gtggttaccgaatatcacgatatggttacggaaatgtactccggtccgtg
cgtcgcgatggaaattcagcaaaacaatgccaccaaaacgtttcgtgaat
tctgtggtccggcagatccggaaatcgcacgtcatctgcgtccgggtacc
ctgcgcgcaatttttggtaaaacgaaaatccagaacgctgtgcactgtac
cgatctgccggaagacggtctgctggaagttcaatactttttcaaaattc
tggataattga
(amino acids)

(SEQ ID NO: 59)

MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFFPSSGGCGPANTAKFTNCT
CCIVKPHAVSEGLLGKILMAIRDAGFEISAMQMFNMDRVNVEEFYEVYKG
VVTEYHDMVTEMYSGPCVAMEIQQNNATKTFREFCGPADPEIARHLRPGT
LRAIFGKTKIQNAVHCTDLPEDGLLEVQYFFKILDN-

Human NME7-AB1 sequence optimized for E. coli expression:

(DNA)

(SEQ ID NO: 60)

Atggaaaaaacgctggccctgattaaaccggatgcaatctccaaagctgg
cgaaattatcgaaattatcaacaaagcgggtttcaccatcacgaaactga
aaatgatgatgctgagccgtaaagaagccctggattttcatgtcgaccac
cagtctcgcccgtttttcaatgaactgattcaattcatcaccacgggtcc
gattatcgcaatggaaattctgcgtgatgacgctatctgcgaatggaaac
gcctgctgggcccggcaaactcaggtgttgcgcgtaccgatgccagtgaa
tccattcgcgctctgtttggcaccgatggtatccgtaatgcagcacatgg
tccggactcattcgcatcggcagctcgtgaaatggaactgtttttcccga
gctctggcggttgcggtccggcaaacaccgccaaatttaccaattgtacg
tgctgtattgtcaaaccgcacgcagtgtcagaaggcctgctgggtaaaat
tctgatggcaatccgtgatgctggctttgaaatctcggccatgcagatgt
tcaacatggaccgcgttaacgtcgaagaattctacgaagtttacaaaggc
gtggttaccgaatatcacgatatggttacggaaatgtactccggtccgtg
cgtcgcgatggaaattcagcaaaacaatgccaccaaaacgtttcgtgaat
tctgtggtccggcagatccggaaatcgcacgtcatctgcgtccgggtacc
ctgcgcgcaatttttggtaaaacgaaaatccagaacgctgtgcactgtac
cgatctgccggaagacggtctgctggaagttcaatactttttctga
(amino acids)

(SEQ ID NO: 61)

MEKTLALIKPDAISKAGEIIEIINKAGFTITKLKMMMLSRKEALDFHVDH
QSRPFFNELIQFITTGPIIAMEILRDDAICEWKRLLGPANSGVARTDASE
SIRALFGTDGIRNAAHGPDSFASAAREMELFFPSSGGCGPANTAKFTNCT
CCIVKPHAVSEGLLGKILMAIRDAGFEISAMQMFNMDRVNVEEFYEVYKG
VVTEYHDMVTEMYSGPCVAMEIQQNNATKTFREFCGPADPEIARHLRPGT
LRAIFGKTKIQNAVHCTDLPEDGLLEVQYFF-

Mouse NME6

(DNA)

(SEQ ID NO: 62)

Atgacctccatcttgcgaagtccccaagctcttcagctcacactagccct
gatcaagcctgatgcagttgcccacccactgatcctggaggctgttcatc
agcagattctgagcaacaagttcctcattgtacgaacgagggaactgcag
tggaagctggaggactgccggaggttttaccgagagcatgaagggcgttt
tttctatcagcggctggtggagttcatgacaagtgggccaatccgagcct
atatccttgcccacaaagatgccatccaactttggaggacactgatggga
cccaccagagtatttcgagcacgctatatagccccagattcaattcgtgg
aagtttgggcctcactgacacccgaaatactacccatggctcagactccg
tggtttccgccagcagagagattgcagccttcttccctgacttcagtgaa
cagcgctggtatgaggaggaggaaccccagctgcggtgtggtcctgtgca
ctacagtccagaggaaggtatccactgtgcagctgaaacaggaggccaca
aacaacctaacaaaacctag
(amino acids)

(SEQ ID NO: 63)

MTSILRSPQALQLTLALIKPDAVAHPLILEAVHQQILSNKFLIVRTRELQ
WKLEDCRRFYREHEGRFFYQRLVEFMTSGPIRAYILAHKDAIQLWRTLMG
PTRVFRARYIAPDSIRGSLGLTDTRNTTHGSDSVVSASREIAAFFPDFSE
QRWYEEEEPQLRCGPVHYSPEEGIHCAAETGGHKQPNKT-

Human NME6:

(DNA)

(SEQ ID NO: 64)

Atgacccagaatctggggagtgagatggcctcaatcttgcgaagccctca
ggctctccagctcactctagccctgatcaagcctgacgcagtcgcccatc
cactgattctggaggctgttcatcagcagattctaagcaacaagttcctg
attgtacgaatgagagaactactgtggagaaaggaagattgccagaggtt
ttaccgagagcatgaagggcgttttttctatcagaggctggtggagttca
tggccagcgggccaatccgagcctacatccttgcccacaaggatgccatc
cagctctggaggacgctcatgggacccaccagagtgttccgagcacgcca
tgtggccccagattctatccgtgggagtttcggcctcactgacacccgca
acaccacccatggttcggactctgtggtttcagccagcagagagattgca
gccttcttccctgacttcagtgaacagcgctggtatgaggaggaagagcc
ccagttgcgctgtggccctgtgtgctatagcccagagggaggtgtccact
atgtagctggaacaggaggcctaggaccagcctga
(amino acids)

(SEQ ID NO: 65)

MTQNLGSEMASILRSPQALQLTLALIKPDAVAHPLILEAVHQQILSNKFL
IVRMRELLWRKEDCQRFYREHEGRFFYQRLVEFMASGPIRAYILAHKDAI
QLWRTLMGPTRVFRARHVAPDSIRGSFGLTDTRNTTHGSDSVVSASREIA
AFFPDFSEQRWYEEEEPQLRCGPVCYSPEGGVHYVAGTGGLGPA-

Human NME6 1:

(DNA)

(SEQ ID NO: 66)

Atgacccagaatctggggagtgagatggcctcaatcttgcgaagccctca
ggctctccagctcactctagccctgatcaagcctgacgcagtcgcccatc
cactgattctggaggctgttcatcagcagattctaagcaacaagttcctg
attgtacgaatgagagaactactgtggagaaaggaagattgccagaggtt
ttaccgagagcatgaagggcgttttttctatcagaggctggtggagttca
tggccagcgggccaatccgagcctacatccttgcccacaaggatgccatc
cagctctggaggacgctcatgggacccaccagagtgttccgagcacgcca
tgtggccccagattctatccgtgggagtttcggcctcactgacacccgca
acaccacccatggttcggactctgtggtttcagccagcagagagattgca
gccttcttccctgacttcagtgaacagcgctggtatgaggaggaagagcc
ccagttgcgctgtggccctgtgtga
(amino acids)

(SEQ ID NO: 67)

MTQNLGSEMASILRSPQALQLTLALIKPDAVAHPLILEAVHQQILSNKFL
IVRMRELLWRKEDCQRFYREHEGRFFYQRLVEFMASGPIRAYILAHKDAI
QLWRTLMGPTRVFRARHVAPDSIRGSFGLTDTRNTTHGSDSVVSASREIA
AFFPDFSEQRWYEEEEPQLRCGPV-

Human NME6 2:

(DNA)

(SEQ ID NO: 68)

Atgctcactctagccctgatcaagcctgacgcagtcgcccatccactgat
tctggaggctgttcatcagcagattctaagcaacaagttcctgattgtac
gaatgagagaactactgtggagaaaggaagattgccagaggttttaccga
gagcatgaagggcgttttttctatcagaggctggtggagttcatggccag
cgggccaatccgagcctacatccttgcccacaaggatgccatccagctct
ggaggacgctcatgggacccaccagagtgttccgagcacgccatgtggcc
ccagattctatccgtgggagtttcggcctcactgacacccgcaacaccac
ccatggttcggactctgtggtttcagccagcagagagattgcagccttct
tccctgacttcagtgaacagcgctggtatgaggaggaagagccccagttg
cgctgtggccctgtgtga
(amino acids)

(SEQ ID NO: 69)

MLTLALIKPDAVAHPLILEAVHQQILSNKFLIVRMRELLWRKED
CQRFYREHEGRFFYQRLVEFMASGPIRAYILAHKDAIQLWRTLMG
PTRVFRARHVAPDSIRGSFGLTDTRNTTHGSDSVVSASREIAAFF
PDFSEQRWYEEEEPQLRCGPV-

Human NME6 3:

(DNA)

(SEQ ID NO: 70)

Atgctcactctagccctgatcaagcctgacgcagtcgcccatccactgat
tctggaggctgttcatcagcagattctaagcaacaagttcctgattgtac
gaatgagagaactactgtggagaaaggaagattgccagaggttttaccga
gagcatgaagggcgttttttctatcagaggctggtggagttcatggccag
cgggccaatccgagcctacatccttgcccacaaggatgccatccagctct
ggaggacgctcatgggacccaccagagtgttccgagcacgccatgtggcc
ccagattctatccgtgggagtttcggcctcactgacacccgcaacaccac
ccatggttcggactctgtggtttcagccagcagagagattgcagccttct
tccctgacttcagtgaacagcgctggtatgaggaggaagagccccagttg
cgctgtggccctgtgtgctatagcccagagggaggtgtccactatgtagc
tggaacaggaggcctaggaccagcctga
(amino acids)

(SEQ ID NO: 71)

MLTLALIKPDAVAHPLILEAVHQQILSNKFLIVRMRELLWRKEDC
QRFYREHEGRFFYQRLVEFMASGPIRAYILAHKDAIQLWRTLMG
PTRVFRARHVAPDSIRGSFGLTDTRNTTHGSDSVVSASREIAAF
FPDFSEQRWYEEEEPQLRCGPVCYSPEGGVHYVAGTGGLGPA-

Human NME6 sequence optimized for E. coli expression:

(DNA)

(SEQ ID NO: 72)

Atgacgcaaaatctgggctcggaaatggcaagtatcctgcgctccccgca
agcactgcaactgaccctggctctgatcaaaccggacgctgttgctcatc
cgctgattctggaagcggtccaccagcaaattctgagcaacaaatttctg
atcgtgcgtatgcgcgaactgctgtggcgtaaagaagattgccagcgttt
ttatcgcgaacatgaaggccgtttcttttatcaacgcctggttgaattca
tggcctctggtccgattcgcgcatatatcctggctcacaaagatgcgatt
cagctgtggcgtaccctgatgggtccgacgcgcgtctttcgtgcacgtca
tgtggcaccggactcaatccgtggctcgttcggtctgaccgatacgcgca
ataccacgcacggtagcgactctgttgttagtgcgtcccgtgaaatcgcg
gcctttttcccggacttctccgaacagcgttggtacgaagaagaagaacc
gcaactgcgctgtggcccggtctgttattctccggaaggtggtgtccatt
atgtggcgggcacgggtggtctgggtccggcatga
(amino acids)

(SEQ ID NO: 73)

MTQNLGSEMASILRSPQALQLTLALIKPDAVAHPLILEAVHQQILS
NKFLIVRMRELLWRKEDCQRFYREHEGRFFYQRLVEFMASGPIRAY
ILAHKDAIQLWRTLMGPTRVFRARHVAPDSIRGSFGLTDTRNTTHG
SDSVVSASREIAAFFPDFSEQRWYEEEEPQLRCGPVCYSPEGGVH
YVAGTGGLGPA-

Human NME6 1 sequence optimized for E. coli expression:

(DNA)

(SEQ ID NO: 74)

Atgacgcaaaatctgggctcggaaatggcaagtatcctgcgctccccgca
agcactgcaactgaccctggctctgatcaaaccggacgctgttgctcatc
cgctgattctggaagcggtccaccagcaaattctgagcaacaaatttctg
atcgtgcgtatgcgcgaactgctgtggcgtaaagaagattgccagcgttt
ttatcgcgaacatgaaggccgtttcttttatcaacgcctggttgaattca
tggcctctggtccgattcgcgcatatatcctggctcacaaagatgcgatt
cagctgtggcgtaccctgatgggtccgacgcgcgtctttcgtgcacgtca
tgtggcaccggactcaatccgtggctcgttcggtctgaccgatacgcgca
ataccacgcacggtagcgactctgttgttagtgcgtcccgtgaaatcgcg
gcctttttcccggacttctccgaacagcgttggtacgaagaagaagaacc
gcaactgcgctgtggcccggtctga
(amino acids)

(SEQ ID NO: 75)

MTQNLGSEMASILRSPQALQLTLALIKPDAVAHPLILEAVHQQILSNKFL
IVRMRELLWRKEDCQRFYREHEGRFFYQRLVEFMASGPIRAYILAHKDAI
QLWRTLMGPTRVFRARHVAPDSIRGSFGLTDTRNTTHGSDSVVSASREIA
AFFPDFSEQRWYEEEEPQLRCGPV-

Human NME6 2 sequence optimized for E. coli expression:

(DNA)

(SEQ ID NO: 76)

Atgctgaccctggctctgatcaaaccggacgctgttgctcatccgctgat
tctggaagcggtccaccagcaaattctgagcaacaaatttctgatcgtgc
gtatgcgcgaactgctgtggcgtaaagaagattgccagcgtttttatcgc
gaacatgaaggccgtttcttttatcaacgcctggttgaattcatggcctc
tggtccgattcgcgcatatatcctggctcacaaagatgcgattcagctgt
ggcgtaccctgatgggtccgacgcgcgtctttcgtgcacgtcatgtggca
ccggactcaatccgtggctcgttcggtctgaccgatacgcgcaataccac
gcacggtagcgactctgttgttagtgcgtcccgtgaaatcgcggcctttt
tcccggacttctccgaacagcgttggtacgaagaagaagaaccgcaactg
cgctgtggcccggtctga
(amino acids)

(SEQ ID NO: 77)

MLTLALIKPDAVAHPLILEAVHQQILSNKFLIVRMRELLWRKEDCQRFYR
EHEGRFFYQRLVEFMASGPIRAYILAHKDAIQLWRTLMGPTRVFRARHVA
PDSIRGSFGLTDTRNTTHGSDSVVSASREIAAFFPDFSEQRWYEEEEPQL
RCGPV-

Human NME6 3 sequence optimized for E. coli expression:

(DNA)

(SEQ ID NO: 78)

Atgctgaccctggctctgatcaaaccggacgctgttgctcatccgctgat
tctggaagcggtccaccagcaaattctgagcaacaaatttctgatcgtgc
gtatgcgcgaactgctgtggcgtaaagaagattgccagcgtttttatcgc
gaacatgaaggccgtttcttttatcaacgcctggttgaattcatggcctc
tggtccgattcgcgcatatatcctggctcacaaagatgcgattcagctgt
ggcgtaccctgatgggtccgacgcgcgtctttcgtgcacgtcatgtggca
ccggactcaatccgtggctcgttcggtctgaccgatacgcgcaataccac
gcacggtagcgactctgttgttagtgcgtcccgtgaaatcgcggcctttt
tcccggacttctccgaacagcgttggtacgaagaagaagaaccgcaactg
cgctgtggcccggtctgttattctccggaaggtggtgtccattatgtggc
gggcacgggtggtctgggtccggcatga
(amino acids)

(SEQ ID NO: 79)

MLTLALIKPDAVAHPLILEAVHQQILSNKFLIVRMRELLWRKEDCQRFYR
EHEGRFFYQRLVEFMASGPIRAYILAHKDAIQLWRTLMGPTRVFRARHVA
PDSIRGSFGLTDTRNTTHGSDSVVSASREIAAFFPDFSEQRWYEEEEPQL
RCGPVCYSPEGGVHYVAGTGGLGPA-

Histidine Tag

(SEQ ID NO: 80)

(ctcgag)caccaccaccaccaccactga

Strept II Tag

(SEQ ID NO: 81)

(accggt)tggagccatcctcagttcgaaaagtaatga

PSMGFR N-10 peptide:

(SEQ ID NO: 82)

QFNQYKTEAASRYNLTISDVSVSDVPFPFSAQSGA

PSMGFR C-10 peptide

(SEQ ID NO: 83)

GTINVHDVETQFNQYKTEAASRYNLTISDVSVSDV

Human NME7

nucleoside diphosphate kinase 7 isoform a [Homo sapiens] (Hu_7)

(SEQ ID NO: 84)

MNHSERFVFIAEWYDPNASLLRRYELLFYPGDGSVEMHDVKNHRTFLKR
TKYDNLHLEDLFIGNKVNVFSRQLVLIDYGDQYTARQLGSRKEKTLALIK
PDAISKAGEIIEIINKAGFTITKLKMMMLSRKEALDFHVDHQSRPFFNEL
IQFITTGPIIAMEILRDDAICEWKRLLGPANSGVARTDASESIRALFGTD
GIRNAAHGPDSFASAAREMELFFPSSGGCGPANTAKFTNCTCCIVKPHAV
SEGLLGKILMAIRDAGFEISAMQMFNMDRVNVEEFYEVYKGVVTEYHDMV
TEMYSGPCVAMEIQQNNATKTFREFCGPADPEIARHLRPGTLRAIFGKTK
IQNAVHCTDLPEDGLLEVQYFFKILDN

Human NME7 isoform a represented by SEQ ID NO:84 has 90.4% sequence identity with Human NME7 isoform b represented by SEQ ID NO:21 in 376 amino acid overlap region.

Human NME7-X1

(DNA)

(SEQ ID NO: 85)

atgatgatgctttcaaggaaagaagcattggattttcatgtagatcacca
gtcaagaccctttttcaatgagctgatccagtttattacaactggtccta
ttattgccatggagattttaagagatgatgctatatgtgaatggaaaaga
ctgctgggacctgcaaactctggagtggcacgcacagatgcttctgaaag
cattagagccctctttggaacagatggcataagaaatgcagcgcatggcc
ctgattcttttgcttctgcggccagagaaatggagttgttttttccttca
agtggaggttgtgggccggcaaacactgctaaatttactaattgtacctg
ttgcattgttaaaccccatgctgtcagtgaaggactgttgggaaagatcc
tgatggctatccgagatgcaggttttgaaatctcagctatgcagatgttc
aatatggatcgggttaatgttgaggaattctatgaagtttataaaggagt
agtgaccgaatatcatgacatggtgacagaaatgtattctggcccttgtg
tagcaatggagattcaacagaataatgctacaaagacatttcgagaattt
tgtggacctgctgatcctgaaattgcccggcatttacgccctggaactct
cagagcaatctttggtaaaactaagatccagaatgctgttcactgtactg
atctgccagaggatggcctattagaggttcaatacttcttcaagatcttg
gataattag
(amino acids)

(SEQ ID NO: 86)

MMMLSRKEALDFHVDHQSRPFFNELIQFITTGPIIAMEILRDDAICEWKR
LLGPANSGVARTDASESIRALFGTDGIRNAAHGPDSFASAAREMELFFPS
SGGCGPANTAKFTNCTCCIVKPHAVSEGLLGKILMAIRDAGFEISAMQMF
NMDRVNVEEFYEVYKGVVTEYHDMVTEMYSGPCVAMEIQQNNATKTFREF
CGPADPEIARHLRPGTLRAIFGKTKIQNAVHCTDLPEDGLLEVQYFFKIL
DN*

Mouse MUC1 and NME7

Mouse MUC1* extracellular domain PSMGFR is 51.1% identical to human in a 45 amino acid overlap region

(SEQ ID NO: 87)

GTFSASDVKSQLIQHKKEA-DDYNLTISEVKVNEMQFPPSAQSRP

Mouse isoform 1 NME7 84.3% identical to human NME7 isoform a in 395 amino acid overlap region

nucleoside diphosphate kinase 7 (mouse NME7) isoform 1 Mus musculus (Mo_7)

(SEQ ID NO: 88)

MRACQQGRSSSLVSPYMAPKNQSERFAFIAEWYDPNASLLRRYELLFYPT
DGSVEMHDVKNRRTFLKRTKYEDLRLEDLFIGNKVNVFSRQLVLIDYGDQ
YTARQLGSRKEKTLALIKPDAVSKAGEIIEMINKSGFTITKLRMMTLTRK
EAADFHVDHHSRPFYNELIQFITSGPVIAMEILRDDAICEWKRLLGPANS
GLSRTDAPGSIRALFGTDGVRNAAHGPDTFASAAREMELFFPSSGGCGPA
NTAKFTNCTCCIIKPHAISEGMLGKILIAIRDACFGMSAIQMFNLDRANV
EEFYEVYKGVVSEYNDMVTELCSGPCVAIEIQQSNPTKTFREFCGPADPE
IARHLRPETLRAIFGKTKVQNAVHCTDLPEDGLLEVQYFFKILDN

Mouse isoform 2 NME7 is 88.4% identical to human NME7 isoform a in a 253 amino acid overlap region

nucleoside diphosphate kinase 7 isoform 2 [Mus musculus] (Mo2-7)

(SEQ ID NO: 89)

MRACQQGRSSSLVSPYMAPKNQSERFAFIAEWYDPNASLLRRYELLFYPT
DGSVEMHDVKNRRTFLKRTKYEDLRLEDLFIGNKVNVFSRQLVLIDYGDQ
YTARQLGSRKEKTLALIKPDAVSKAGEIIEMINKSGFTITKLRMMTLTRK
EAADFHVDHHSRPFYNELIQFITSGPVIAMEILRDDAICEWKRLLGPANS
GLSRTDAPGSIRALFGTDGVRNAAHGPDTFASAAREMELFFPSSGGCGPA
NTAKFTNCTCCIIKPHAISEDLFIHYM

Pig Sus scrofa MUC1 and NME7

(SEQ ID NO: 90)

MTRDIQAPFFFGLLLLPVLTGEGNKQTNKNLALSLSSQFLQVYKEDGLLG
LFYIKFRPGSVLVELILAFQDSAAAHNLKTQFDRLKAEAGTYNLTISEVS
VIDAPFPSSAQPGSGVPGWGIALLVLVCILVALAIIYVIALAVCQCRRKN
CGQLDIFPTRDAYHPMSEYPTYHTHGRYVPPGSTKRNPYEQVSAGNGGGS
LSYSNLAATSANL

Pig MUC1* PSMGFR is 52.2% identical to human in a 46 amino acid overlap

(SEQ ID NO: 91)

QDSAAAHNLKTQFDRLKAEAGTYNLTISEVSVIDAPFPSSAQPGS

Pig NME7 is 65.6% identical to human NME7 isoform a in a 453 amino acid overlap region

PREDICTED: nucleoside diphosphate kinase 7 [Sus scrofa, Pig] (Pi_7)

(SEQ ID NO: 92)

MNHSERFVFIAEWYDPNASLFRRYELLFYPGDGSVEMHDVKNHRTFLKRT
KYEDLHLEDLFIGNKVNVFSRQLVLIDYGDQYTARQLGSKKEKTLALIKP
DAVSKAGEIIEIINKAGFTLTKLKMMTLSRKEATDFHIDHQSRPFLNELI
QFITSGPIIAMEILRDDAICEWKKLLGPANSGLARTDAPGSIRAVFGTDG
IRNAAHGPDSLSCAAREMELFFPSSGVCGPANTAKFTNCTTCCIVKPHAI
SEGLLGKILMAIRDAGFEISAMQMFNMDRVNVEEFYEVYKGVVSEYNEMV
TEMYFSAPSSSAIWRSPTVLNSLQSDISSRDFSSGPRSIPRSNFYWLTNH
LLEMLSLLLLGVHKGVPKEVFVGEAHVSPGCAPVLVGGTLSRVKDRKKEN
HFSLVFVMLSSVSLPASSRYVKAAKGPQLIKGFSRGRGLLLALNTGCGNC
FWL

Sheep MUC1 and NME7

(SEQ ID NO: 93)

MTPDIQAPFLSLLLLFQVLTVANVTMLTASVSTSPNSTVQVSSTQSSPTS
SPTKETSWSTTTTLLRTSSPAPTPTTSPGRDGASSPTSSAAPSPAASSSH
DGALSLTGSPAPSPTASPGHGGTLTTTSSPAPSPTASPGHDGASTPTSSP
APSPAASPGHDGALSLTGSPAPSPTASPGHGGTLTTTSSPAPSPTASPGH
DGASTPTSSPAPSPAASSSHDGALSLTGSPAPSPPASPGHGGTLTTTSSP
APSPTASPGHGGTLTTTSSPAPSPTASPGHDGASTPTSSPAPTAHSSHDG
ALTTTGSPAPSPAASPGHDSVPPRATSPAPSPAASPGQHAASSPTSSDIS
SVTTSSMSSSMVTSAHKGTSSRATTTPVSKGTPSSVPSSETAPTAASHST
RTAAASTSPSTALSTASHPKTSQQLSVQVSLFFLSFRITNLQFNSSLENP
QTSYYQELQRSILDVILQTYKQRDFLGLSEIKFRPGSVLVDLTLAFREGT
TAELVKAQFSQLEAHAANYSLTISGVSVRDAQFPSSAPSASGVPGWGIAL
LVLVCVLVALAIIYLIALVVCQCGRKKCEQLDIFPTLGAYHPMSEYSAYH
THGRFVPPGSTKRSPYEEVSAGNGGSNLSYTNLAATSANL

Sheep MUC1* extracellular domain PSMGFR is 46.8% identical to human

(SEQ ID NO: 94)

REGTTAELVKAQFSQLEAHAANYSLTISGVSVRDAQFPSSAPSAS

Sheep NME7 is 88.4% identical to human NME7 isoform a in a 395 amino acid overlap region

PREDICTED: nucleoside diphosphate kinase 7 isoform X1 [Ovis aries, Sheep] (Sh_7)

(SEQ ID NO: 95)

MNPTFVLLSLERNVTESLGNHSERFVFIAEWFDPNASLFRRYELLFYPGD
GSVEMHDVKNHRTFLKRTKYEDLHLEDLFIGNKVNIFSRQLVLLDYGDQY
TARQLGSRKEKTLALIKPDAVSKAGEIIEIINKAGFTLTKLKMMTLSRKE
ATDFHIDHQSRPFLNELIQFITSGPIIAMEILRDDAICEWKRLLGPANSG
LARTDAPESIRALFGTDGIKNAAHGPDSFACAAREMELFFPSSGVCGPAN
TAKFTNCTTCCIVKPHAVSEGLLGKILITIRDAGFEISAMQMFNMDRINV
EEFYEVYKGVVSEYNEMVTEMYSGPCVAMEIQQTNPTMTFREFCGPADPE
IARHLRPGTLRAIFGKTKIQNAVHCTDLPEDGLLEVQYFFKILDN

Crab-eating Macque Macaca fascicularis MUC1 and NME7

Crab-eating macaque MUC1* extracellular domain PSMGFR is 88.9% identical to human

(SEQ ID NO: 96)

GTTNVHDVETQFNQRKTEAASRYNLTISDISVRDVPFPFSAQTGA

Crab-eating Macque NME7 is 98% identical to human NME7 isoform a in a 251 amino acid overlap

unnamed protein product [Macaca fascicularis] (Ma_7) (sequence incomplete, only NME7A)

(SEQ ID NO: 97)

MSHSERFVFIAEWYDPNASLLRRYELLFYPGDGSVEMHDVKNHRTFLKRT
KYDSLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITSGPVIAMEILRDDAICEWKRLLGPANSGVARTDASGSIRALFGTDG
IRNAAHGPDSFASAAREMELFFPSSGGCGPANTAKFTNCTCCIVKPHAVS
EVRRNP

Rhesus macaque MUC1 and NME7

Rhesus macque MUC1* PSMGFR

Rhesus macaque MUC1* extracellular domain PSMGFR is 88.9% identical to human

(SEQ ID NO: 98)

GTTNVHDVETQFNQRKTEAASRYNLTISDISVRDVPFPFSAQTGA

Rhesus Macaque NME7 is 98.4% identical to human NME7 isoform a in a 376 amino acid overlap region

Macaca mulatta (Rhesus macaque) (Mm_7)

(SEQ ID NO: 99)

MSHSERFVFIAEWYDPNASLLRRYELLFYPGDGSVEMHDVKNHRTFLKRT
KYDSLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITSGPVIAMEILRDDAICEWKRLLGPANSGVARTDASGSIRALFGTDG
IRNAAHGPDSFASAAREMELFFPSSGGCGPANTAKFTNCTCCIVKPHAVS
EGLLGKILMAIRDAGFEISAMQMFNMDRVNVEEFYEVYKGVVTEYHDMVT
EMYSGPCVAMEIQQNNATKTFREFCGPVDPEIARHLRPGTLRAIFGKTKI
QNAVHCTDLPEDGLLEVQYFFKILDN

Bonobo MUC1 and NME7

Bonobo Pan paniscus MUC1

(SEQ ID NO: 100)

MTPGVQSPFFLLLLLTVLTATTAPKPATVVTGSGHASSAPGGEKETSATQ
RSSVPSSTEKNAVSMTSSVLSSHSPGSGSSTTQGQDVTLAPATEPASGSA
ATWGQDVTSVPVTRPALGSTTPPAHDVTSALDNKPAPGSTAPPAHDVTSA
PDTRPAPGSTAPPAHGVTSAPDTRPALGSTAPPVHNVTSASGSASGSAST
LVHNGTSARATTTPASKSTPFSIPSHHSDTPTTLASHSTKTDASSTHHST
VPPLTSSNHSTSPQLSTGVSFFFLSFHISNLRFNSSLEDPSTDYYQELQR
DISEMFLQIYKQGGFLGLSNIKFRPGSVVVQLTLAFREGTINVHDVETQF
NQYKTEAASRYNLTISDVSVSDVPFPFSAQSGAGVPGWGIALLVLVCVLV
ALAIVYLIALAVCQCRRKNYGQLDIFPARDTYHPMSEYPTYHTHGRYVPP
SSTDRSPYEKVSAGNGGSSLSYTNPAVAATSANL

Bonobo MUC1* extracellular domain PSMGFR is 100% identical to human.

(SEQ ID NO: 101)

GTINVHDVETQFNQYKTEAASRYNLTISDVSVSDVPFPFSAQSGA

Bonobo NME7 100% identical to human NME7 isoform a in a 376 amino acid overlap region

Bonobo PREDICTED: nucleoside diphosphate kinase 7 [Pan paniscus, Bonobo] (Bo_7)

(SEQ ID NO: 102)

MNHSERFVFIAEWYDPNASLLRRYELLFYPGDGSVEMHDVKNHRTFLKRT
KYDNLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITTGPIIAMEILRDDAICEWKRLLGPANSGVARTDASESIRALFGTDG
IRNAAHGPDSFASAAREMELFFPSSGGCGPANTAKFTNCTCCIVKPHAVS
EGLLGKILMAIRDAGFEISAMQMFNMDRVNVEEFYEVYKGVVTEYHDMVT
EMYSGPCVAMEIQQNNATKTFREFCGPADPEIARHLRPGTLRAIFGKTKI
QNAVHCTDLPEDGLLEVQYFFKILDN

Chimpanzee MUC1 and NME7

Pan troglodytes MUC1

(SEQ ID NO: 103)

MTPGIQSPFFLLLLLTVLTVVTGSGHASSAPGGEKETSATQRSSVPSSTE
KNAVSMTSSVLSSHSPGSGSSTTQGQDVTLAPATEPASGSAATWGQDVTS
VPVTRPALGSTTPPAHDVTSAPDNKPAPGSTAPPAHDVTSAPDTRPAPGS
TAPPAHGVTSAPDTRPALGSTAPPVHNVTSASGSASGSASTLVHNGTSAR
ATTTPASKSTPFSIPSHHSDTPTTLASHSTKTDASSTHHSTVPPLTSSNH
STSPQLSTGVSFFFLSFHISNLRFNSSLEDPSTDYYQELQRDISEMFLQI
YKQGGFLGLSNIKFRPGSVVVQLTLAFREGTINVHDVETQFNQYKTEAAS
RYNLTISDVSVSDVPFPFSAQSGAGVPGWGIALLVLVCVLVALAIVYLIA
LAVCQCRRKNYGQLDIFPARDTYHPMSEYPTYHTHGRYVPPSSTDRSPYE
KVSAGNGGSSLSYTNPAVAATSANL

Chimpanzee MUC1* extracellular domain PSMGFR is 100% identical to human

(SEQ ID NO: 104)

GTINVHDVETQFNQYKTEAASRYNLTISDVSVSDVPFPFSAQSGA

Chimpanzee NME7 is 99.7% identical to human NME7 isoform a in a 376 amino acid overlap region

nucleoside diphosphate kinase 7 [Pan troglodytes, Chimp] (CH_7)

(SEQ ID NO: 105)

MNHSERFVFIAEWYDPNASLLRRYELLFYPGDGSVEMHDVKNHRTFLKRT
KYDNLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITTGPIIAMEILRDDAICEWKRLLGPANSGVARTDASESIRALFGTDG
IRNAAHGPDSFASAAREMELFFPSSGGCGPANTAKFTNCTCCIVKPHAVS
EGLLGKILMAIRDAGFEISAMQMFNMDRVNVEEFYEVYKGVVTEYHNMVT
EMYSGPCVAMEIQQNNATKTFREFCGPADPEIARHLRPGTLRAIFGKTKI
QNAVHCTDLPEDGLLEVQYFFKILDN

Gorilla MUC1 and NME7

Gorilla gorilla MUC1

(SEQ ID NO: 106)

MTPGTQSPFFLLLLLTVLTATTAPKPTTVVTGSGHASSTPGGEKETSATQ
RSSVPSSTEKNAVSMTSSILSSHSPGSGSSTTQGQDVTPAPATEPASGSA
ATWGQDVTSVPVTRPALGSTTPPAHDVTSAPDNKPAPGSTTPPAHGVSSA
PDTRPAPGSTAPPAHGVTSAPDTRPAPGSTAPPAHVHNVTSASGSASGSA
STLVHNGTSARATTTPASKSTPFSIPSHHSDTPTTLANHSTKTDASSTHH
STVPPLTSSNHSTSPQLSTGVSFFFLSFHISNLQFNSSLEDPSTDYYQEL
QRDISEMFLQIYKQGGFLGLSNIKFRPGSVVVQLTLAFREGTINVHDVET
QFNQYKTEAASRYNLTISDVSVSDVPFPFSAQSGAGVPGWGIALLVLVCV
LVVLAIVYLIALAVCQCRRKNYGQLDIFPVRDTYHPMSEYPTYHTHGRYV
PPSSTDRSPYEKVSAGNGGSSLSYTNPAVAATSANL

Gorilla MUC1* PSMGFR is 100% identical to human

(SEQ ID NO: 107)

GTINVHDVETQFNQYKTEAASRYNLTISDVSVSDVPFPFSAQSGA

Gorilla NME7 is 78.2% identical to human NME7 isoform a in a 376 amino acid overlap region

NME7 [ENSGGOP00000002464], Gorilla gorilla (Go_7)

(SEQ ID NO: 108)

MNHSERFVFIAEWYDPNASLLRRYELLFYPGDGSVEMHDVKNHRTFLKRT
KYDNLHLEDLFIGNKVNVFSRQLVLIDYGDQYTARQLGSRKEKTLALIKP
DAISKAGEIIEIINKAGFTITKLKMMMLSRKEALDFHVDHQSRPFFNELI
QFITTGPIIAMEILRDDAICEWKRLLGPANSGVARTDASESIRALFGTDG
IRNAAHGPDSFASAARLLGKILMAIRDAGFEISAMQMFNMDRVNVEEFYE
VYKGVVTEYHDMVTEMYSGPCVAMEIQQNNATKTFREFCGPADP

Mouse

nucleoside diphosphate kinase 7 [Mus musculus] (Mo_7)

(SEQ ID NO: 109)

MKTNQSERFAFIAEWYDPNASLLRRYELLFYPTDGSVEMHDVKNRRTFL
KRTKYEDLRLEDLFIGNKVNVFSRQLVLIDYGDQYTARQLGSRKEKTLAL
IKPDAVSKAGEIIEMINKSGFTITKLRMMTLTRKEAADFHVDHHSRPFYN
ELIQFITSGPVIAMEILRDDAICEWKRLLGPANSGLSRTDAPGSIRALFG
TDGVRNAAHSPDTFASAAREMELFFPSSGGCGPANTAKFTNCTCCIIKPH
AISEGMLGKILIAIRDACFGMSAIQMFNLDRANVEEFYEVYKGVVSEYND
MVTELCSGPCVAIEIQQSNPTKTFREFCGPADPEIARHLRPETLRAIFGK
TKVQNAVHCTDLPEDGLLEVQYFFKILDN

Mouse NME7 is 87.8% identical to human NME7 isoform a in a 378 amino acid overlap region

nucleoside diphosphate kinase 7 isoform X1 [Mus musculus] (MoX1-7)

(SEQ ID NO: 110)

MHDVKNRRTFLKRTKYEDLRLEDLFIGNKVNVFSRQLVLIDYGDQYTAR
QLGSRKEKTLALIKPDAVSKAGEIIEMINKSGFTITKLRMMTLTRKEAAD
FHVDHHSRPFYNELIQFITSGPVIAMEILRDDAICEWKRLLGPANSGLSR
TDAPGSIRALFGTDGVRNAAHGPDTFASAAREMELFFPSSGGCGPANTAK
FTNCTCCIIKPHAISEGMLGKILIAIRDACFGMSAIQMFNLDRANVEEFY
EVYKGVVSEYNDMVTELCSGPCVAIEIQQSNPTKTFREFCGPADPEIARH
LRPETLRAIFGKTKVQNAVHCTDLPEDGLLEVQYFFKILDN

Mouse NME7 is 79.8% identical to human NME7 isoform a in a 376 amino acid overlap region

Macaca fascicularis

unnamed protein product [Macaca fascicularis] (Ma_7) (sequence incomplete, only NME7A)

(SEQ ID NO: 111)

MSHSERFVFIAEWYDPNASLLRRYELLFYPGDGSVEMHDVKNHRTFLKR
TKYDSLHLEDLFIGNKVNVFSRQLVLIDYGDQYTARQLGSRKEKTLALIK
PDAISKAGEIIEIINKAGFTITKLKMMMLSRKEALDFHVDHQSRPFFNEL
IQFITSGPVIAMEILRDDAICEWKRLLGPANSGVARTDASGSIRALFGTD
GIRNAAHGPDSFASAAREMELFFPSSGGCGPANTAKFTNCTCCIVKPHAV
SEVRRNP

Macaca NME7 is 98.0% identical to human NME7 isoform a in a 251 amino acid overlap region

Macaca mulatta (Rhesus macaque) (Mm_7)

(SEQ ID NO: 112)

MSHSERFVFIAEWYDPNASLLRRYELLFYPGDGSVEMHDVKNHRTFLKR
TKYDSLHLEDLFIGNKVNVFSRQLVLIDYGDQYTARQLGSRKEKTLALIK
PDAISKAGEIIEIINKAGFTITKLKMMMLSRKEALDFHVDHQSRPFFNEL
IQFITSGPVIAMEILRDDAICEWKRLLGPANSGVARTDASGSIRALFGTD
GIRNAAHGPDSFASAAREMELFFPSSGGCGPANTAKFTNCTCCIVKPHAV
SEGLLGKILMAIRDAGFEISAMQMFNMDRVNVEEFYEVYKGVVTEYHDMV
TEMYSGPCVAMEIQQNNATKTFREFCGPVDPEIARHLRPGTLRAIFGKTK
IQNAVHCTDLPEDGLLEVQYFFKILDN

Macaca NME7 is 98.4% identical to human NME7 isoform a in a 376 amino acid overlap region

Chimp

nucleoside diphosphate kinase 7 b [Pan troglodytes, Chimp] (CHb_7)

(SEQ ID NO: 113)

MHDVKNHRTFLKRTKYDNLHLEDLFIGNKVNVFSRQLVLIDYGDQYTAR
QLGSRKEKTLALIKPDAISKAGEIIEIINKAGFTITKLKMMMLSRKEALD
FHVDHQSRPFFNELIQFITTGPIIAMEILRDDAICEWKRLLGPANSGVAR
TDASESIRALFGTDGIRNAAHGPDSFASAAREMELFFPSSGGCGPANTAK
FTNCTCCIVKPHAVSEGLLGKILMAIRDAGFEISAMQMFNMDRVNVEEFY
EVYKGVVTEYHDMVTEMYSGPCVAMEIQQNNATKTFREFCGPADPEIARH
SRPGTLRAIFGKTKIQNAVHCTDLPEDGLLEVQYFFKILDN

Chimp NME7 is 90.2% identical to human NME7 isoform a in a 376 amino acid overlap region

Ovis aries

PREDICTED: nucleoside diphosphate kinase 7 isoform X2 (Shx2_7) [Ovis aries, Sheep]

(SEQ ID NO: 114)

MNHSERFVFIAEWFDPNASLFRRYELLFYPGDGSVEMHDVKNHRTFLKRT
KYEDLHLEDLFIGNKVNIFSRQLVLLDYGDQYTARQLGSRKEKTLALIKP
DAVSKAGEIIEIINKAGFTLTKLKMMTLSRKEATDFHIDHQSRPFLNELI
QFITSGPIIAMEILRDDAICEWKRLLGPANSGLARTDAPESIRALFGTDG
IKNAAHGPDSFACAAREMELFFPSSGVCGPANTAKFTNCTTCCIVKPHAV
SEGLLGKILITIRDAGFEISAMQMFNMDRINVEEFYEVYKGVVSEYNEMV
TEMYSGPCVAMEIQQTNPTMTFREFCGPADPEIARHLRPGTLRAIFGKTK
IQNAVHCTDLPEDGLLEVQYFFKILDN

Sheep NME7 is 92.6% identical to human NME7 isoform a in a 377 amino acid overlap region

PREDICTED: nucleoside diphosphate kinase 7 isoform X3 [Ovis aries, Sheep] (Shx3_7)

(SEQ ID NO: 115)

MHDVKNHRTFLKRTKYEDLHLEDLFIGNKVNIFSRQLVLLDYGDQYTAR
QLGSRKEKTLALIKPDAVSKAGEIIEIINKAGFTLTKLKMMTLSRKEATD
FHIDHQSRPFLNELIQFITSGPIIAMEILRDDAICEWKRLLGPANSGLAR
TDAPESIRALFGTDGIKNAAHGPDSFACAAREMELFFPSSGVCGPANTAK
FTNCTTCCIVKPHAVSEGLLGKILITIRDAGFEISAMQMFNMDRINVEEF
YEVYKGVVSEYNEMVTEMYSGPCVAMEIQQTNPTMTFREFCGPADPEIAR
HLRPGTLRAIFGKTKIQNAVHCTDLPEDGLLEVQYFFKILDN

Sheep NME7 is 83.6% identical to human NME7 isoform a in a 377 amino acid overlap region

Mouse

Mouse MUC1

(SEQ ID NO: 116)

MTPGIRAPFFLLLLLASLKGFLALPSEENSVTSSQDTSSSLASTTTPVHS
SNSDPATRPPGDSTSSPVQSSTSSPATRAPEDSTSTAVLSGTSSPATTAP
VNSASSPVAHGDTSSPATSLSKDSNSSPVVHSGTSSAATTAPVDSTSSPV
VHGGTSSPATSPPGDSTSSPDHSSTSSPATRAPEDSTSTAVLSGTSSPAT
TAPVDSTSSPVAHDDTSSPATSLSEDSASSPVAHGGTSSPATSPLRDSTS
SPVHSSASIQNIKTTSDLASTPDHNGTSVTTTSSALGSATSPDHSGTSTT
TNSSESVLATTPVYSSMPFSTTKVTSGSAIIPDHNGSSVLPTSSVLGSAT
SLVYNTSAIATTPVSNGTQPSVPSQYPVSPTMATTSSHSTIASSSYYSTV
PFSTFSSNSSPQLSVGVSFFFLSFYIQNHPFNSSLEDPSSNYYQELKRNI
SGLFLQIFNGDFLGISSIKFRSGSVVVESTVVFREGTFSASDVKSQLIQH
KKEADDYNLTISEVKVNEMQFPPSAQSRPGVPGWGIALLVLVCILVALAI
VYFLALAVCQCRRKSYGQLDIFPTQDTYHPMSEYPTYHTHGRYVPPGSTK
RSPYEEVSAGNGSSSLSYTNPAVVTTSANL

Macaca mulatta (rhesus macaque) MUC1, partial

(SEQ ID NO: 117)

VTGSGHTNSTPGGEKETSATQRSSMPISTKNAVSMTSRLSSHSPVSGSST
TQGQDVTLALATEPATGSATTLGHNVTSAPDTSAAPGSTGPPAGVVTSAP
DTSAAPGSTGPPARVVTSAPDTSAAPGSTGPPARVVTSAPDTSAAPGSTG
PPARVVTSAPDTSAAPGSTGPPARVVTSAPDTSAAPGSTGPPARVVTSAP
DTSAAPGSTGPPARVVTSAPDTSAAPGSTGPPARVVTSAPDTSAAPGSTG
PPARVVTSAPGTSAAPGSTAPPGSTAPPAHDVTSASDSASGSASTLVHST
TSARATTTPASKSTPFSIPSHHSDTPTTLASHSTKTDASSTHHSTVPPFT
SNHSTSPQLSLGVSFFFLSFHISNLQFNSSLEDPSTNYYQQLQRDISELF
LQIYKQGDFLGLSNIMFRPGSVVVQSTLVFREGTTNVHDVETQFNQRKTE
AASRYNLTISDISVRDVPFPFSAQTGAGVPGWGIALLVLVCVLVVLAIVY
FIALAVCQCRQKNYRQLDIFPARDAYHPMSEYPTYHTHGRYVPAGGTNRS
PYE

Macaca fascicularis_MUC1 (isoform X1)

(SEQ ID NO: 118)

MTPGTQSPFFLLLILTVLTAATVPEPTTVVTGSGHTNSTPGGEKETSATQ
RSSMPISTKNAVSMTSRLSSHSPVSGSSTTQGQDVTLALAMESATGSATT
LGHVVTSAPDTSAAPGSTGPPAHVVTSAPDTSAAPGSTGPPAHVVTSAPD
TSAAPGSTAPPAHVVTSAPDTSAAPGSTAPPAHDVTSASDSASGSASTLV
HSTTSARATTTPASKSTPFSIPSHHSDTPTTLASHSTKTDASSTHHSTVP
PFTSSNHSTSPQLSLGVSFFFLSFHISNLQFNSSLEDPSTNYYQQLQRDI
SELFLQIYKQGDFLGLSNIMFRPGSVVVQSTLVFREGTTNVHDVETQFNQ
RKTEAASRYNLTISDISVRDVPFPFSAQTGAGVPGWGIALLVLVCVLVVM
AIVYFIALAVCQCRQKNYRQLDIFPARDAYHPMSEYPTYHTHGRYAPAGG
TNRSPYEEVSAGNGGSSLSYTNPAVAATSANL

Sus scrofa MUC1, partial

(SEQ ID NO: 119)

NSSLEDPTTSYYKDLQRRISELFLQVYKEDGLLGLFYIKFRPGSVLVELI
LAFQDSAAAHNLKTQFDRLKAEAGTYNLTISEVSVIDAPFPSSAQPGSGV
PGWGIALLVLVCILVALAIIYVIALAVCQCRRKNCGQLDIFPTRDAYHPM
SEYPTYHTHGRYVPPGSTKRNPYEQVSAGNGGGSLSYSNLAATSANL

Ovis aries MUC1 (Sheep)

(SEQ ID NO: 120)

MTPDIQAPFLSLLLLFQVLTVANVTMLTASVSTSPNSTVQVSSTQSSPTS
SPTKETSWSTTTTLLRTSSPAPTPTTSPGRDGASSPTSSAAPSPAASSSH
DGALSLTGSPAPSPTASPGHGGTLTTTSSPAPSPTASPGHDGASTPTSSP
APSPAASPGHDGALSLTGSPAPSPTASPGHGGTLTTTSSPAPSPTASPGH
DGASTPTSSPAPSPAASSSHDGALSLTGSPAPSPPASPGHGGTLTTTSSP
APSPTASPGHGGTLTTTSSPAPSPTASPGHDGASTPTSSPAPTAHSSHDG
ALTTTGSPAPSPAASPGHDSVPPRATSPAPSPAASPGQHAASSPTSSDIS
SVTTSSMSSSMVTSAHKGTSSRATTTPVSKGTPSSVPSSETAPTAASHST
RTAAASTSPSTALSTASHPKTSQQLSVQVSLFFLSFRITNLQFNSSLENP
QTSYYQELQRSILDVILQTYKQRDFLGLSEIKFRPGSVLVDLTLAFREGT
TAELVKAQFSQLEAHAANYSLTISGVSVRDAQFPSSAPSASGVPGWGIAL
LVLVCVLVALAIIYLIALVVCQCGRKKCEQLDIFPTLGAYHPMSEYSAYH
THGRFVPPGSTKRSPYEEVSAGNGGSNLSYTNLAATSANL

I. In one aspect, the present invention is directed to a method of testing for efficacy or toxicity of a potential drug agent in a chimeric animal that expresses some human DNA or some human tissues. In this method, an animal that expresses some human DNA or tissues is generated by introducing human naïve state stem cells into a non-human cell or cells. In one aspect the non-human cell is an egg, in another aspect it is a fertilized egg, in another aspect, the cells are a morula, blastocyst or embryo. For ethical concerns or other reasons, it may be advantageous to generate chimeric animals wherein the integrating naïve state stem cells are also non-human, but of a different species than the recipient cell, cells, morula, blastocyst or embryo. In the method above, the agent that maintains stem cells in the naïve state or reverts primed stem cells to the naïve state may be an NME protein, 2i, 5i, or other cocktails of inhibitors, chemicals, or nucleic acids. The NME protein may be NME1 dimer, NME7 monomer, NME7-AB, NME7-X1, NME6 dimer, or bacterial NME.

The non-human mammal may be a rodent, such as a mouse or rat, primate, including macaque, rhesus monkey, ape, chimp, bonobo and the like, or a domestic animal including pig, sheep, bovine, and the like. The chimeric animal may have a genetic disorder, have an induced disease, or a cancer that may be spontaneously generated or implanted from cells derived from a human being.

In the method above, the non-human animal may be transgenic, wherein the animal expresses human MUC1 or MUC1* or NME protein in the germ cells or somatic cells, wherein the germ cells and somatic cells contain a recombinant human MUC1 or MUC1* or NME gene sequence introduced into said animal. The gene expressing the human MUC1 or MUC1* or NME protein may be under control of an inducible promoter. The promoter may be inducibly responsive to a naturally occurring protein in the non-human animal or an agent that can be administered to the animal before, after or during development. Alternatively, the non-human animal may be transgenic, wherein the animal expresses its native sequence MUC1 or MUC1* or NME protein in the germ cells or somatic cells, wherein the germ cells and somatic cells contain a recombinant native species MUC1 or MUC1* or NME gene sequence introduced into said mammal. The NME species can be NME7, NME7-X1, NME1, NME6 or a bacterial NME.

In the method above, the agent that maintains stem cells in the naïve state or reverts primed stem cells to the naïve state may be an NME protein, 2i, 5i, or other cocktails of inhibitors, chemicals, or nucleic acids. The NME protein may be NME1 dimer, NME7 monomer, NME7-AB, NME7-X1, NME6 dimer, or bacterial NME.

In this method, the agent may suppress expression of MBD3, CHD4, BRD4 or JMJD6. The agent may be siRNA made against MBD3, CHD4, BRD4 or JMJD6, or siRNA made against any gene that encodes a protein that upregulates expression of MBD3, CHD4, BRD4 or JMJD6. The cancer stem cell may be characterized by increased expression of CXCR4 or E-cadherin (CDH1) compared with cancer cells or normal cells.

In another aspect, the invention is directed to a method for generating tissue from xenograft in a non-human mammal, comprising: (i) generating a transgenic non-human mammal, wherein the mammal expresses human MUC1 or MUC1* or NME protein in the germ cells and somatic cells, wherein the germ cells and somatic cells contain a recombinant human MUC1 or MUC1* or NME gene sequence introduced into said mammal, wherein the expression of the gene sequence may be under control of an inducible and repressible regulatory sequence; (ii) transferring stem cells or progenitor cells that are xenogeneic in origin to the non-human mammal such that the gene may be induced to be expressed so as to multiply the number of stem or progenitor cells; and (iii) repressing the gene expression so as to generate tissue from the xenografted stem cells.

In this method, in step (iii), the gene expression repression may be carried out by contacting the stem cells with a tissue differentiation factor, or in step (iii) the gene expression repression may be carried out naturally in the mammal in response to naturally produced host tissue differentiation factor. The transferred cells may be human. The tissue may be an organ. The NME protein may be NME7, NME7-AB, NME7-X1, NME1, NME6, or bacterial NME. The animal may be a mammal, a rodent, a primate or domesticated animal such as a pig, sheep, or bovine species.

II. In other aspects, the present invention is directed to making animals having at least some human cells or cells in which at least some of the DNA is of human origin. Such animals would grow human tissue, tissue containing some human cells or cells containing some human DNA for the generation of human or human-like tissue. In other cases such animals would grow organs comprising at least some human cells. In other cases such animals would grow organs comprised entirely of human cells. In yet other cases, host animals can be genetically or molecularly manipulated even after development to grow human limbs. Limbs, nerves, blood vessels, tissues, organs, or factors made in them, or secreted from them, would then be harvested from the animals and used for a multitude of purposes including but not limited to: 1) transplant into humans; 2) administration into humans for medicinal benefit, including anti-aging; and 3) scientific experiments including drug testing and disease modeling.

In one aspect, the invention is directed to a method for generating human tissues in a non-human animal comprising: (i) generating human naïve state stem cells and injecting them into a fertilized egg, morula, blastocyst or embryo of a non-human animal such that a chimeric animal is generated; (ii) harvesting human tissues, organs, cells or factors secreted by or made in the human tissues or cells from the chimeric animal; and (iii) transplanting or administering the harvested material into a human. The naïve state stem cells may be generated using NME7, NME7-AB, NME7-X1 or dimeric NME1. The naïve stem cells may be iPS cells that have been reprogrammed in a medium containing NME7, NME7-AB, NME7-X1 or dimeric NME1. Or, the naïve stem cells may be embryonic stem cells that have been cultured in a medium containing NME7, NME7-AB, NME7-X1 or dimeric NME1. The non-human cells of the blastocyst or embryo may have been genetically altered. And the genetic alteration may result in the host animal being unable to generate a certain tissue or organ. The genetic alteration may be to make the non-human animal express human molecules that facilitate or enhance the incorporation or growth of human stem or progenitor cells in the non-human host animal. Further, the agent that maintains stem cells in the naïve state or reverts primed stem cells to the naïve state may be an NME protein, 2i, 5i, chemical, or nucleic acid. The NME protein may be NME1 dimer, NME7 monomer, NME7-AB, NME6 dimer, or bacterial NME, or NME7-X1. The non-human animal may be a rodent, mouse, rat, pig, sheep, non-human primate, macaque, chimpanzee, bonobo, gorilla or any non-human mammal. In one aspect of the invention, the non-human animal is chosen for its high sequence homology to human NME protein, especially human NME7-AB or NME7-X1 or high sequence homology to human MUC1* extracellular domain. In some cases, the NME protein may be present in serum-free media as the single growth factor.

One test of whether or not a chimeric animal can be generated is if stem cells from a first species are able to incorporate into the inner cell mass (ICM) of a second species. Chimeric animals are more readily generated when the two different species are closely related, for example two rodents. We injected human naïve state stem cells into a mouse morula and showed they incorporated into the inner cell mass. In a specific example, human naïve state stem cells that had been generated in human NME7-AB, then cultured in human, were injected into a mouse morula 2.5 days after fertilization of the egg. This is before the inner cell mass forms. Forty-eight (48) hours later, the morula was analyzed and such analysis showed that the human stem cells had incorporated into the inner cell mass, indicating that a chimeric animal will develop.

III. In one aspect, the present is directed to a method for generating human tissues or organ in a non-human animal host comprising: (i) generating human naïve state stem cells and injecting them into a fertilized egg, morula, blastocyst, embryo or developing fetus of the non-human animal host such that a chimeric animal is generated; (ii) harvesting human tissues, organs, cells or factors secreted by or made in the human tissues or cells from the chimeric animal; (iii) transplanting or administering the harvested material into a human resulting in generation of human tissues. The naïve state stem cells are generated using NME7, NME7-AB, NME7-X1, NME6 or dimeric NME1. The naïve stem cells are iPS cells that have been reprogrammed in a medium containing NME7, NME7-AB, NME6, NME7-X1 or dimeric NME1. The naïve stem cells are embryonic stem cells that have been cultured in a medium containing NME7, NME7-AB, NME6, NME7-X1 or dimeric NME1. Non-human cells of the blastocyst or embryo have been genetically altered. The genetic alteration results in the host animal being unable to generate a certain tissue or organ. The agent that maintains stem cells in the naïve state or reverts primed stem cells to the naïve state is an NME protein, 2i, 5i, chemical, or nucleic acid. The NME protein is NME1 dimer, NME7 monomer, NME7-AB, NME6 dimer, or bacterial NME. The non-human animal is a rodent, pig bovine, sheep or primate. The rodent is a mouse or rat. The NME protein is present in serum free media as the single growth factor. The non-human animal host expresses NME protein having a sequence that is homologous to the native sequence of the species of the stem cells to be generated. The NME protein is NME7, NME7-AB, NME7-X1, or dimeric NME1 or NME6. The NME protein is NME7. The NME protein is at least 45%, 50%, 55%, 60%, 65%, 70%, 75%, 80%, 85%, 90%, or 95% homologous to the native NME protein sequence of the species of the stem cells to be generated. The NME protein is at least 60% homologous to the native sequence of the species of the stem cells to be generated. The NME protein is at least 70% homologous to the native sequence of the species of the stem cells to be generated.

Other aspects of the invention are directed to: an NME protein having a sequence at least 75% homologous to native mouse NME protein, an NME protein having a sequence at least 75% homologous to native rat NME protein, an NME protein having a sequence at least 75% homologous to native pig NME protein, an NME protein having a sequence at least 75% homologous to native sheep NME protein, an NME protein having a sequence at least 75% homologous to native bovine NME protein, an NME protein having a sequence at least 75% homologous to native crab-eating macaque NME protein, an NME protein having a sequence at least 75% homologous to native rhesus monkey NME protein, an NME protein having a sequence at least 75% homologous to native chimpanzee NME protein, an NME protein having a sequence at least 75% homologous to native bonobo NME protein, an NME protein having a sequence at least 75% homologous to native gorilla NME protein, an antibody that binds to a peptide comprising the sequence of the extracellular domain of MUC1*, wherein the sequence is non-human, an antibody that binds to a peptide comprising the sequence of the extracellular domain of MUC1*, wherein the sequence is primate, an antibody that binds to a peptide comprising the sequence of the extracellular domain of MUC1*, wherein the sequence is macaque, chimpanzee, ape, bonobo, or gorilla, an antibody that binds to a peptide comprising the sequence of the extracellular domain of MUC1*, wherein the sequence is non-primate, an antibody that binds to a peptide comprising the sequence of the extracellular domain of MUC1*, wherein the sequence is rodent, an antibody that binds to a peptide comprising the sequence of the extracellular domain of MUC1*, wherein the sequence is mouse or rat, an antibody that binds to a peptide comprising the sequence of the extracellular domain of MUC1*, wherein the sequence is mammalian, an antibody that binds to a peptide comprising the sequence of the extracellular domain of MUC1*, wherein the sequence is pig, bovine, or sheep.

In another aspect, the invention is directed to a method for generating stem cells, inducing pluripotency in somatic cells or culturing stem cells comprising the steps of contacting cells with an NME protein and/or an anti-MUC1* antibody wherein the NME protein is at least 75% homologous to the sequence of the donor cells and the anti-MUC1* antibody binds to a peptide comprising the sequence of a MUC1* extracellular domain wherein the sequence is at least 75% homologous to the native sequence of the species that donated the cells.

In another aspect, the invention is directed to a method of treating a person in need of generated tissue or organ, comprising carrying out the steps described above.

In yet another aspect, the invention is directe to a method of generating a first non-human mammal that comprises DNA, molecules, cells, tissue or organ specifically originating from a second mammal that does or does not belong to the same species or genus as the first non-human mammal, comprising introducing cells from the second mammal into the first non-human mammal. The cells from the second mammal are progenitor cells, stem cells or naïve state stem cells. The naïve state stem cells are generated by culturing cells in a media that contains NME. The NME is dimeric NME1, dimeric NME6, NME7-X1 or NME7-AB. The NME has sequence endogenous to the second mammal. The second mammal is human. The first non-human mammal is a rodent, a domesticated mammal, pig, bovine, or a non-human primate. The progenitor cells, stem cells or naïve stem cells are introduced into the fertilized egg, morula, blastocyst, embryo or developing fetus of the first non-human mammal.

In the above described methods, further steps include: allowing the first non-human mammal to develop and harvesting from the first non-human mammal molecules, cells, tissues or organs that have incorporated some second mammalian DNA; and administering to the second mammal in need thereof the molecules, cells, tissues or organs for the treatment or prevention of a disease or condition. The progenitor cells, stem cells or naïve stem cells are iPS cells. The somatic cells from which the iPS cells are generated are from the second mammal to which the obtained molecules, cells, tissues or organs for the treatment or prevention of a disease or condition is administered.

In the above described methods, further steps include: determining an organ developmental time period and endogenous genes involved in the development of the organ; and knocking out or knocking down the endogenous gene during the developmental time period of the organ in the first non-human mammal, wherein the organ is caused to be produced from the cells from the second mammal.

In the above methods, the first non-human mammal is close to the second mammal with global sequence identity that is greater than 70%, 75%, 80%, 85%, 90%, or 95% or NME sequence identity that is greater than 45%, 50%, 55%, 60%, 65%, 70%, 75%, 80%, 85%, 90%, or 95%.

In the above described methods, further steps include: determining an organ developmental time period and endogenous genes involved in the development of the organ; and genetically altering the fertilized egg, cells of morula, cells of the blastocyst, or cells of the embryo or developing fetus of the first non-human mammal such that second mammalian NME7-AB or NME1 is expressed from an inducible or repressable promoter such that the second mammalian cells are timely expanded in response to the non-mammalian NME7-AB or NME1. Further steps include injecting the second mammalian stem cells into embryo at a later stage of development at the location where the desired organ or tissue would normally develop. Further steps include expanding the mammalian stem cells by inducing expression of either first non-human mammalian or second mammalian NME7 or NME1 at that location. Further steps include expanding the mammalian stem cells by inducing expression of either first non-human mammalian or second mammalian NME1 at that location. A second mammalian promoter is linked to an endogenous first non-human mammalian protein and is expressed at a desired time and location, then introducing an agent that directs the development of the desired tissue. The endogenous first non-human mammalian protein is a protein that induces expression of NME1 or NME7, preferably NME1.

In another aspect, the invention is directed to a method of testing for efficacy or toxicity of a potential drug agent in a chimeric animal that expresses some second mammalian DNA or some second mammalian tissue, comprising: (i) generating a first non-human mammal that comprises DNA, molecules, cells, tissue or organ specifically originating from a second mammal that does or does not belong to the same species or genus as the first non-human mammal, comprising introducing cells from the second mammal into the first non-human mammal; and (ii) administering a test drug to the first non-human mammal for the effect on the tissue or organ originating from the second mammal. NME is expressed in the first non-human mammal that enhances proliferation of the cells originating from the second mammal.

In another aspect, the invention is directed to a method discovering a potential drug agent in a chimeric animal that expresses some second mammal DNA or some second mammal tissues, comprising: (i) generating a first non-human mammal that comprises DNA, molecules, cells, tissue or organ specifically originating from a second mammal that does or does not belong to the same species or genus as the first non-human mammal, comprising introducing cells from the second mammal into the first non-human mammal; and (ii) administering a compound to the first non-human mammal for the effect on the tissue or organ originating from the second mammal, wherein efficacious effects indicate that the present of a potential drug. NME is expressed in the first non-human mammal that enhances proliferation of the cells originating from the second mammal.

MUC1*/NME in Stem Cell Proliferation and Induction

We discovered that human stem cells overexpress MUC1* which is a potent growth factor receptor. The ligand of MUC1*, NM23-H1, also called NME1, in dimeric form is alone sufficient to make human stem cells grow in the pluripotent state, without the need for feeder cells, conditioned media, or any other growth factors or cytokines (Mahanta S et al 2008; Hikita S et al 2008). We previously showed that NME1 dimers are ligands of the MUC1* growth factor receptor, wherein MUC1* is the remaining transmembrane portion after most of the extra cellular domain has been cleaved and shed from the cell surface. The remaining portion of the extra cellular domain is comprised essentially of the PSMGFR sequence. NME1 dimers bind to and dimerize the MUC1* receptor. Competitive inhibition of the NME-MUC1* interaction, using a synthetic PSMGFR peptide, induced differentiation of pluripotent stem cells, which shows that pluripotent stem cell growth is mediated by the interaction between NME1 dimers and MUC1* growth factor receptor. Human stem cells secrete NME1 where, after dimerization, it binds to the MUC1* receptor and stimulates growth and pluripotency of human stem cells. Competitive inhibition of the interaction by either addition of the PSMGFR peptide or by adding the Fab of an anti-MUC1* (anti-PSMGFR) antibody induced cell death and differentiation (Hikita et al 2008) in vitro.

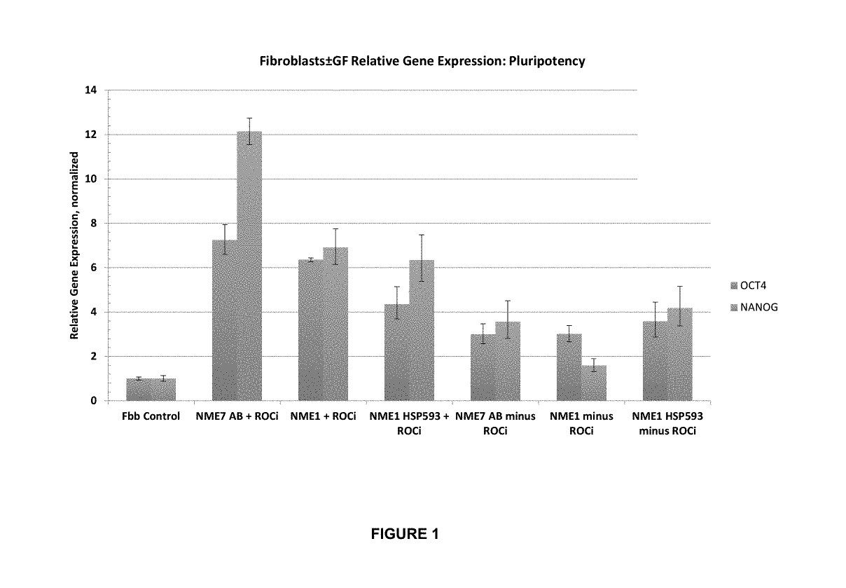

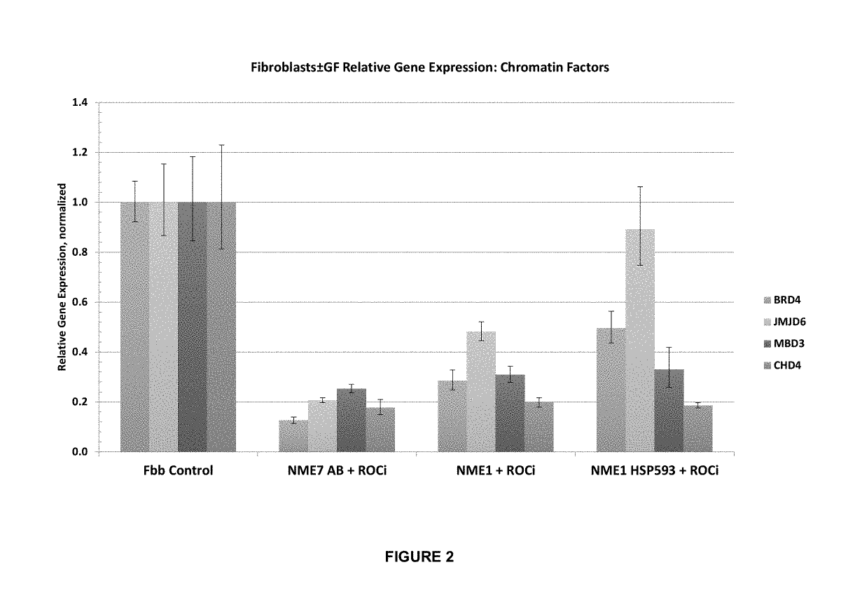

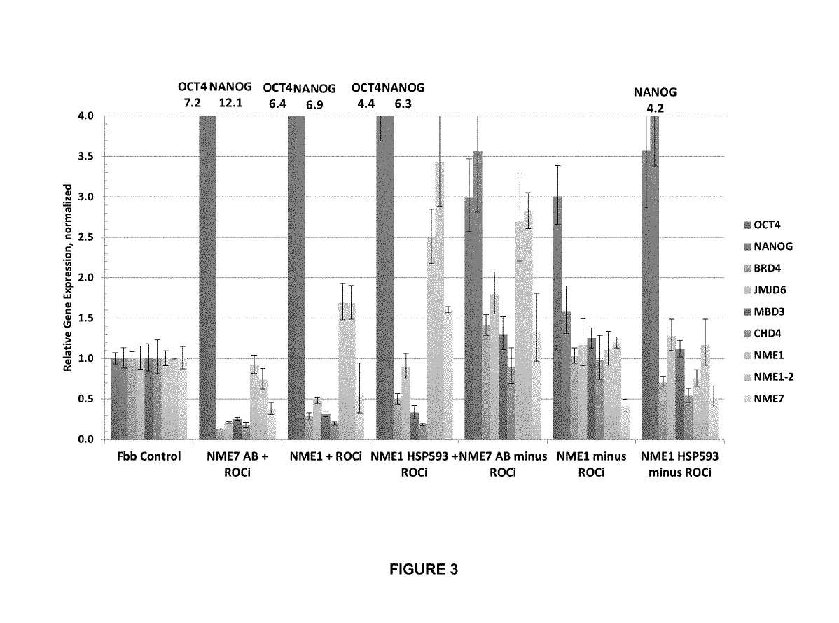

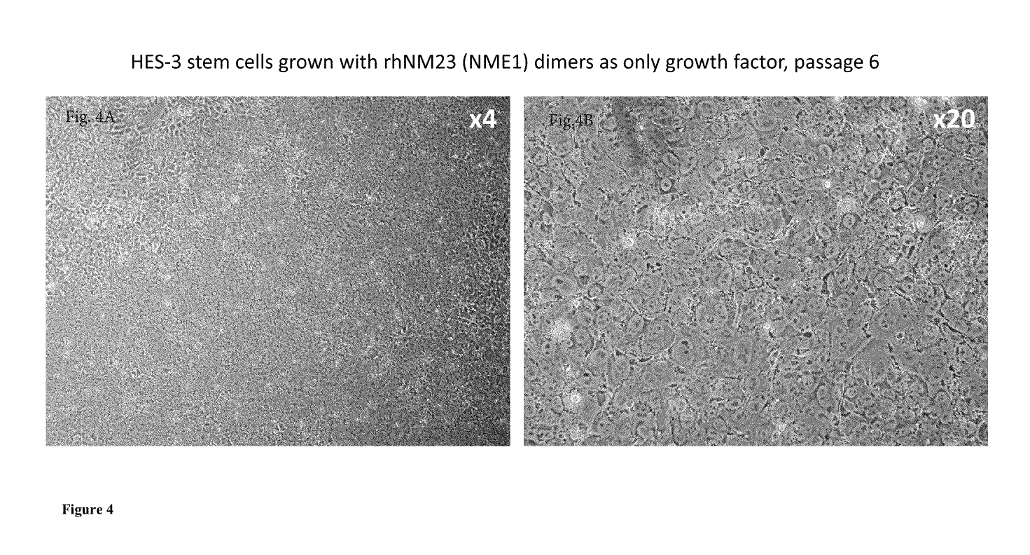

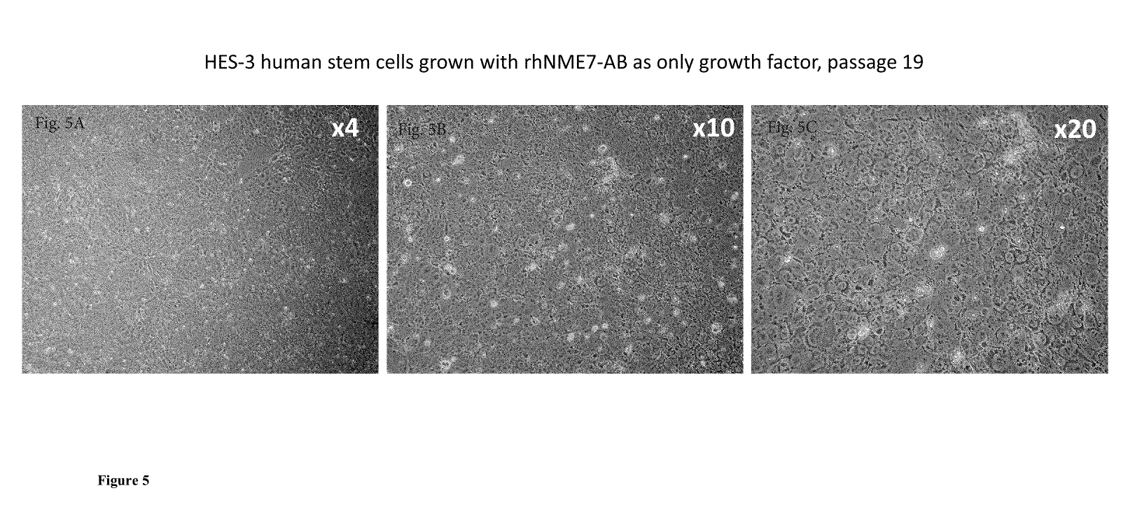

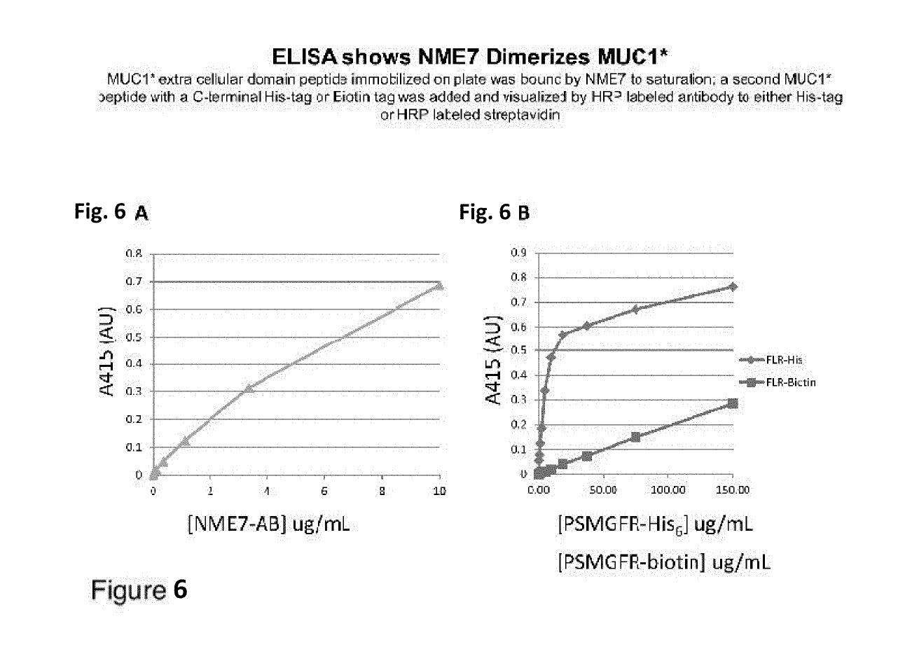

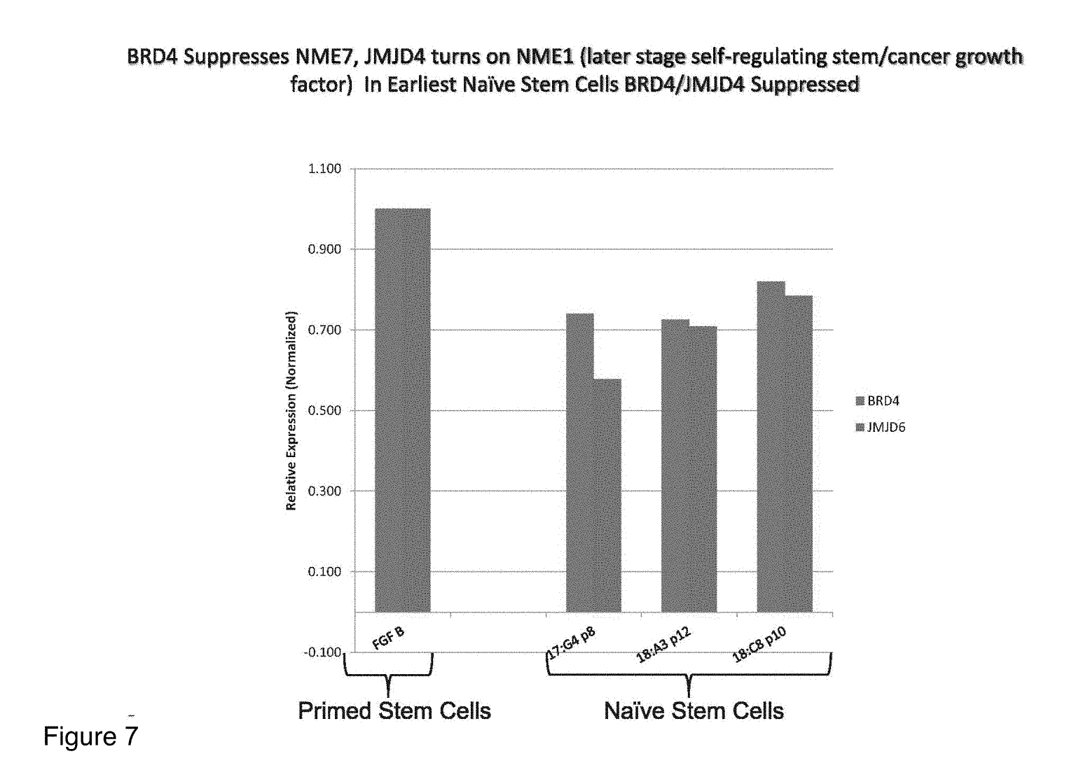

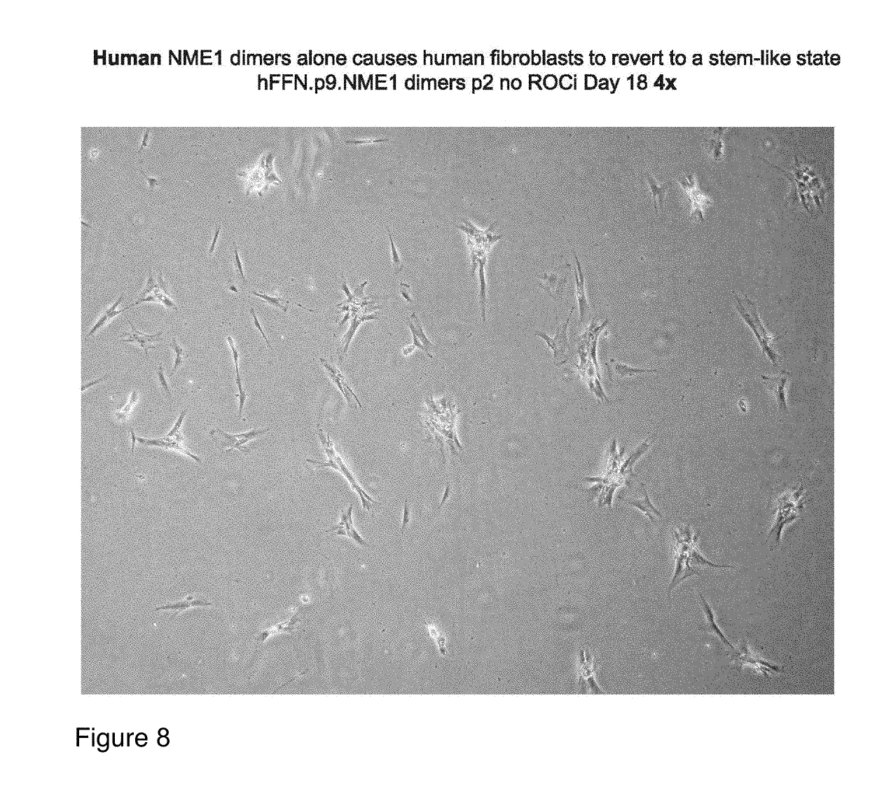

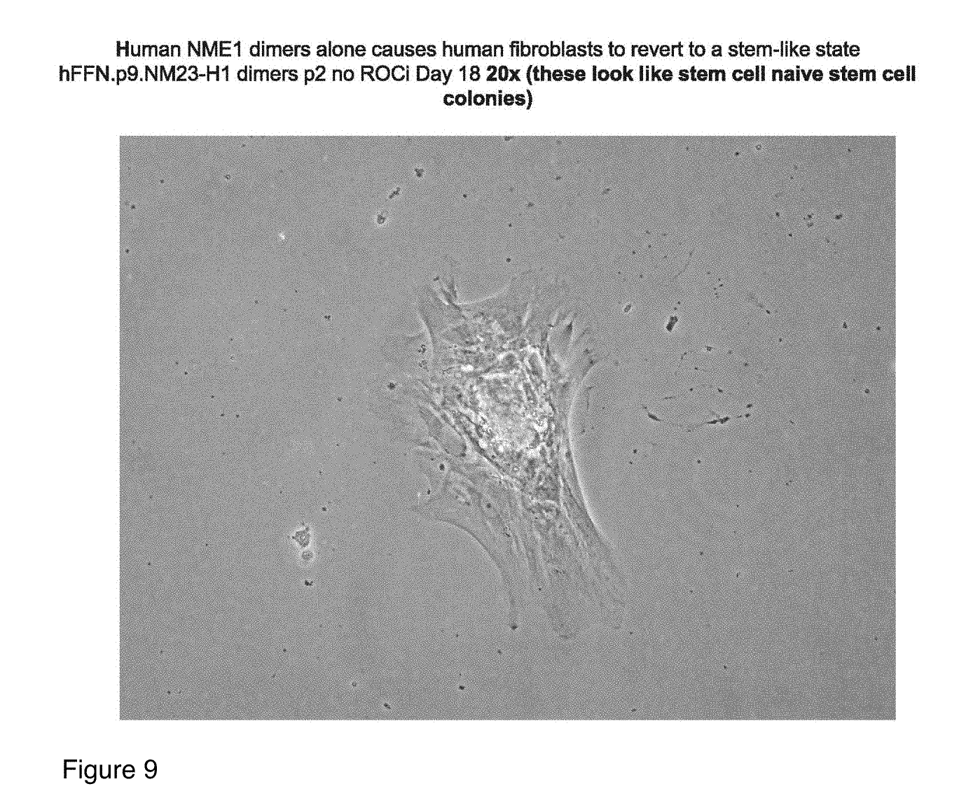

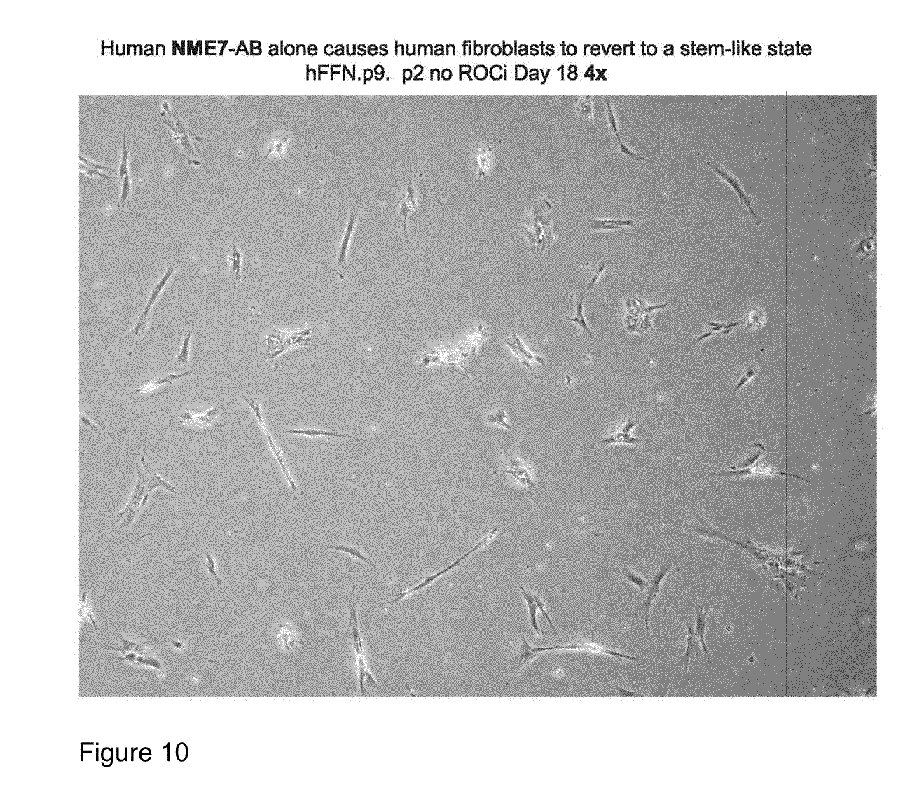

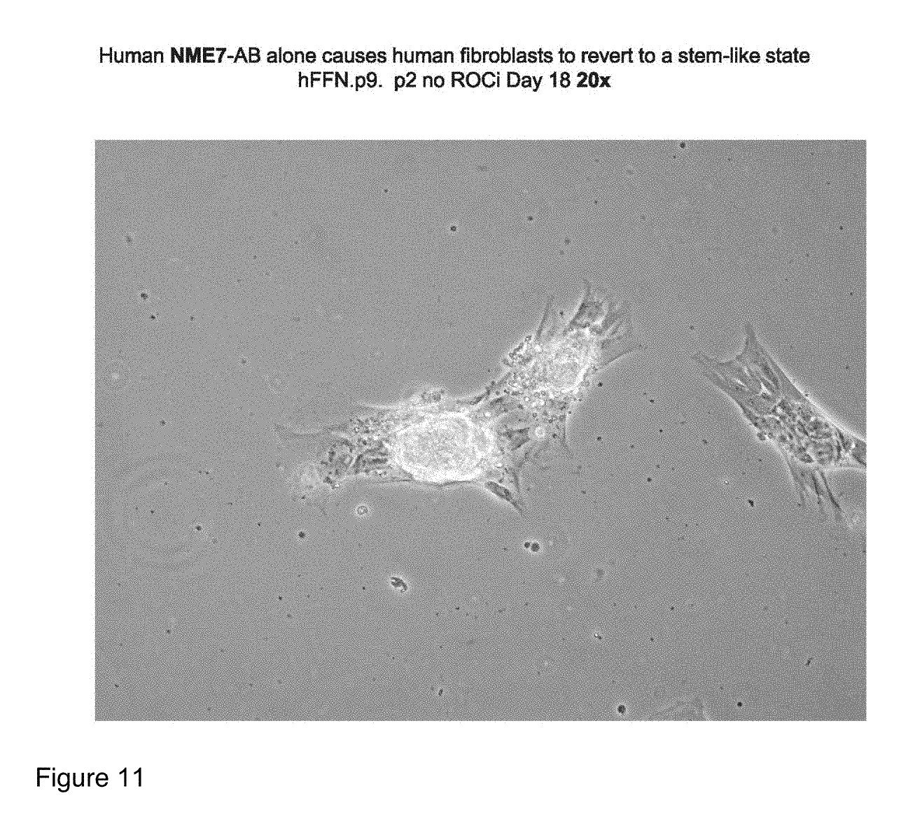

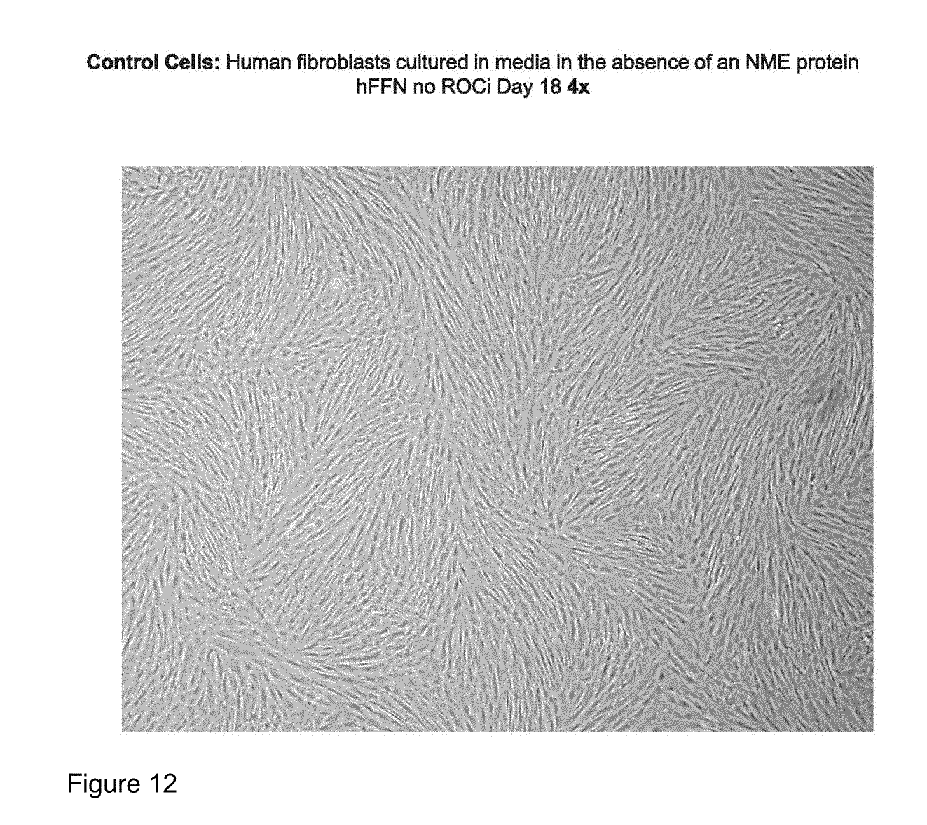

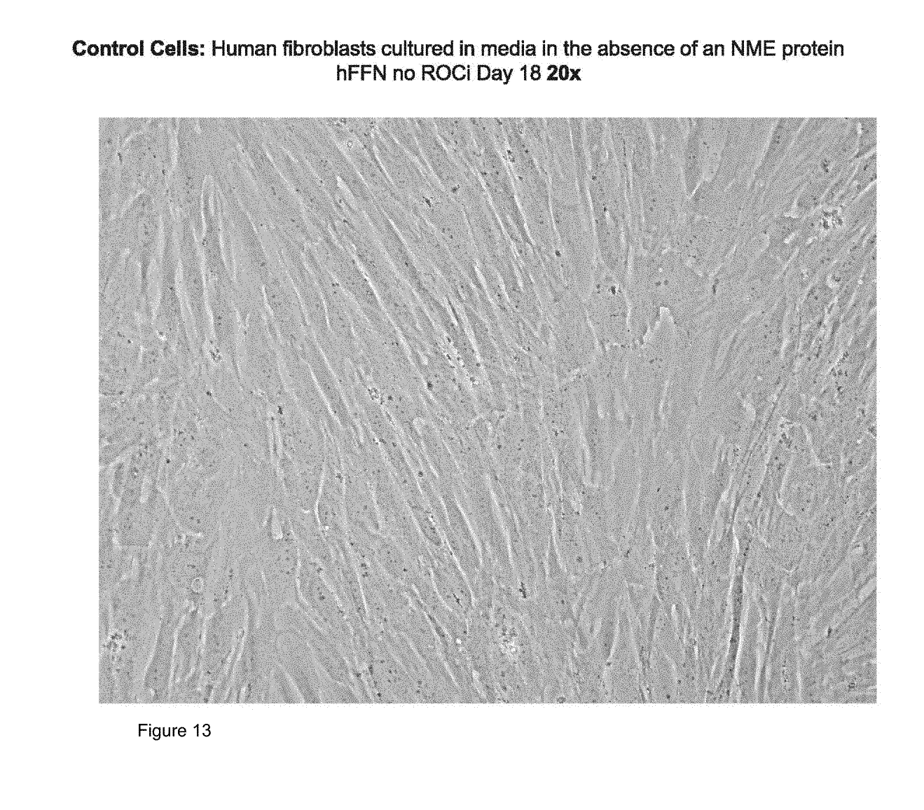

We discovered a new growth factor of the NME family, NME7, that makes human stem cells grow and inhibits their differentiation, in the absence of FGF or any other growth factor. We made a truncated, recombinant human NME7 that we call NME7-AB or rhMNE7-AB. It is devoid of the N-terminal DM10 domain and has a molecular weight of approximately 33 kDa. A naturally occurring NME7 cleavage product that is secreted from stem cells appears to be essentially the same as our recombinant NME7-AB. An alternative splice variant of NME7 called NME7-X1 was theorized. We showed by PCR that NME7-X1 does exist in nature and is also secreted by human stem cells and is 30 kDa. We have also made a human recombinant NME7-X1. In addition, we showed that some bacterial NME1 proteins that have high sequence homology to human NME1 act the same as human NME1. They bind to and dimerize the MUC1* extracellular domain and induce pluripotency. One such bacterial NME1 protein that we showed supports human stem cell growth and induces pluripotency is Halomonas Sp. 593, also known as HSP593. Fibroblasts are somatic cells, not stem cells. However, we found that NME7-AB, NME7-X1, NME1 dimers and HSP593 dimers are able to induce somatic cells to revert to a less mature state. FIG. 1 shows that simply culturing human fibroblasts in NME7-AB, NME1 dimers or HSP593 NME1 dimers causes upregulation of stem cell markers OCT4 and NANOG. FIG. 2 shows that these NME proteins suppress expression of MBD3 and CHD4; suppression of these genes was previously shown to make human stem cells revert to a more naïve state (Rais Y et al, 2013). BRD4 has been shown to suppress expression of NME7 and its co-factor JMJD6 upregulates NME1. The PCR graph of FIG. 3 shows that NME7-AB or NME1 dimers induce pluripotency by upregulating pluripotency genes and suppressing those that others have reported are suppressed in naïve state stem cells. FIGS. 4A and 4B show that culturing human stem cells in NME1 dimers as the only added growth factor fully supports pluripotent stem cell growth. FIGS. 5A-5C show that culturing human stem cells in NME7-AB monomers as the only added growth factor fully supports pluripotent stem cell growth. Whereas NME1 must be a dimer in order to bind to and dimerize the MUC1* growth factor receptor, monomeric NME7-AB and NME7-X1 have two binding sites for MUC1*. FIGS. 6A and 6B show that in a sandwich ELISA assay, NME7-AB is able to simultaneously bind to two MUC1* extracellular domain peptides, also referred to herein as PSMGFR peptides (JHK SEQ ID?). BRD4 and co-factor JMJD6 are suppressed in naïve state stem cells as is shown in FIG. 2 and FIG. 7. FIGS. 8-11 show that NME1 dimers and NME7-AB induce human fibroblasts to revert to a less mature, stem-like state as can be seen by their dramatic change in morphology that resembles stem cell morphology and is without question unlike fibroblast morphology which is shown in FIGS. 12-13.

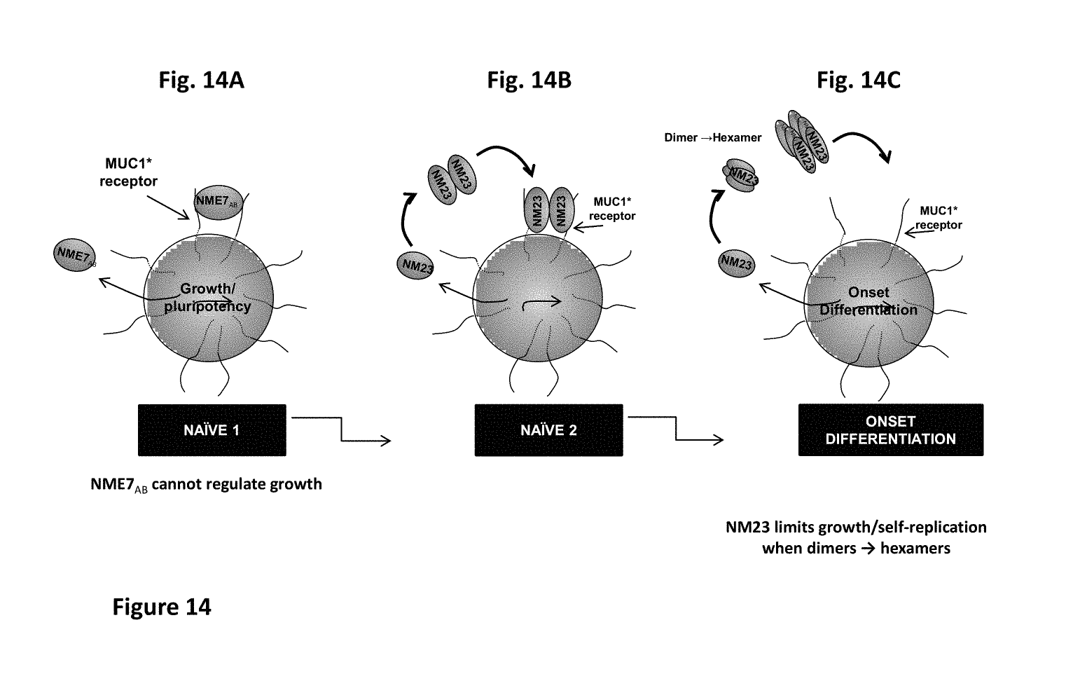

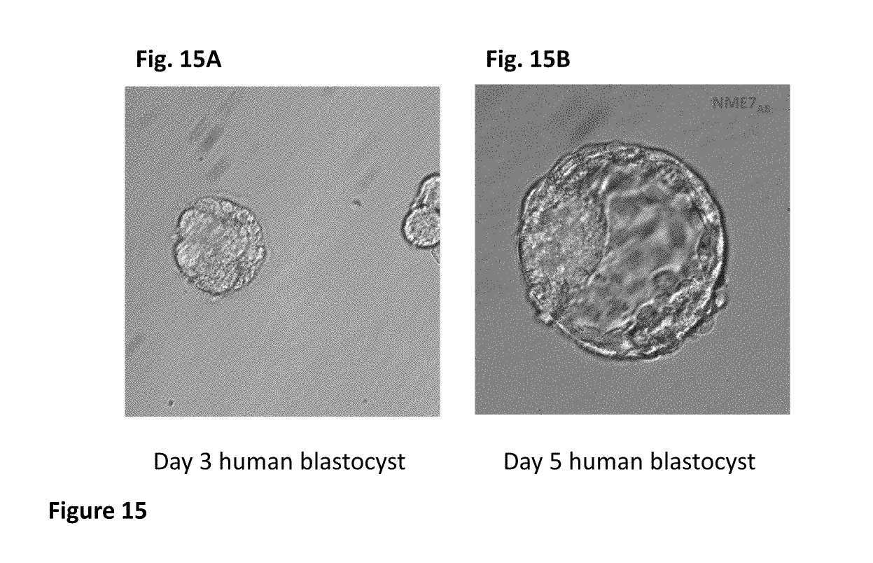

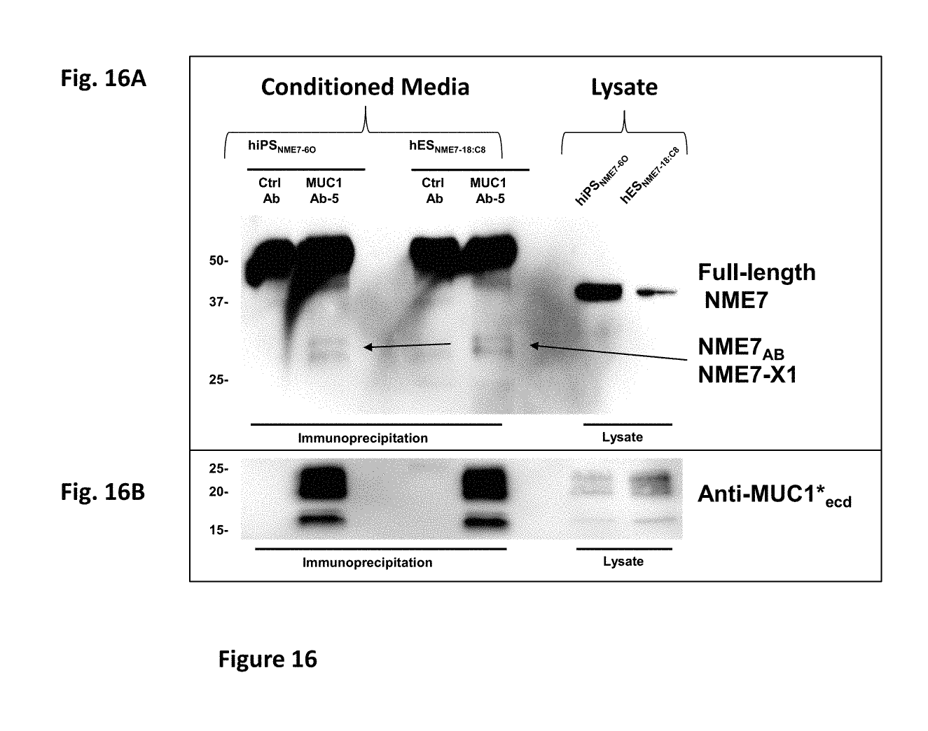

We theorized that in the very earliest human stem cells, NME7-AB and NME7-X1 are secreted which allows them to bind to and dimerize the extracellular domain of MUC1* growth factor receptor. Thus, NME7-AB stabilizes a first naïve state. At a later stage, BRD4 suppresses NME7 and its co-factor JMJD6 upregulates expression of NME1 that must be a dimer to bind to and dimerize the MUC1* growth factor receptor. Thus, NME1 dimers stabilize a second naïve-like state. As the stem cells of a developing morula or blastocyst multiply, the amount of NME1 that is secreted increases and the dimers become hexamers. Hexameric NME1 does not bind to MUC1* and induces differentiation (Smagghe et al 2013). FIG. 14 shows this mechanistic model of how stem cells limit self-replication. Previously, NME7 was only reported to be expressed in testis. We discovered that early cells of a human morula and the inner cell mass of human blastocysts express NME7. FIG. 15A shows that all the cells of a Day 3 human morula stained positive for NME7 (say which antibody in example section #61). FIG. 15B shows that by Day 5 the morula has developed into a blastocyst and at this stage of development, the NME7-positive cells are restricted to the inner cell mass, which are known to be in naïve state. We discovered that naïve state human stem cells express and secrete two truncated forms of NME7 that are both devoid of the N-terminal DM10 domain. These truncated NME7 species bind to the extracellular domain of MUC1* growth factor receptor. One NME7 form that we call NME7-AB has undergone post-translational cleavage to produce an NME7 species that runs with an apparent molecular weight of ˜33 kDa. The other truncated form is an alternative splice isoform called NME7-X1 that is ˜30 kDa. This is in contrast to full-length NME7 that has a calculated molecular weight of ˜42 kDa and which appears to be restricted to the cytoplasm. NME1 dimers, NME6 dimers, NME7-AB and NME7-X1 function as a growth factors for human stem cells. They promote growth, pluripotency and induction of naïve state by binding to and dimerizing the MUC1* growth factor receptor. However, NME7-AB and NME7-X1 do so as monomers as they have two binding sites for the extracellular domain of MUC1*. FIG. 16A-16B shows photographs of Western blot gels from a co-immunoprecipitation experiment in which human naive state induced pluripotent stem (iPS) cells and embryonic stem (ES) cells were lysed and an antibody against the cytoplasmic tail of MUC1 (Ab5) was used to co-immunoprecipitate species that bind to MUC1. The immunoprecipitates were then assayed by Western blot. FIG. 16A shows a photograph of the Western blot that was probed with an anti-NME7 antibody and shows two NME7 species, one with molecular weight of 30 kDa and the other 33 kDa, bound to MUC1, whereas full-length NME7 in crude cell lysate has molecular weight of 42 kDa, and FIG. 16B shows a photograph of the Western blot wherein the gel of FIG. 16A was stripped and re-probed with an anti-MUC1* extracellular domain antibody, showing that NME7-AB or NME7-X1 bound to the cleaved form of MUC1 called MUC1* that runs with a molecular weight of 17-25 kDa, depending on glycosylation.

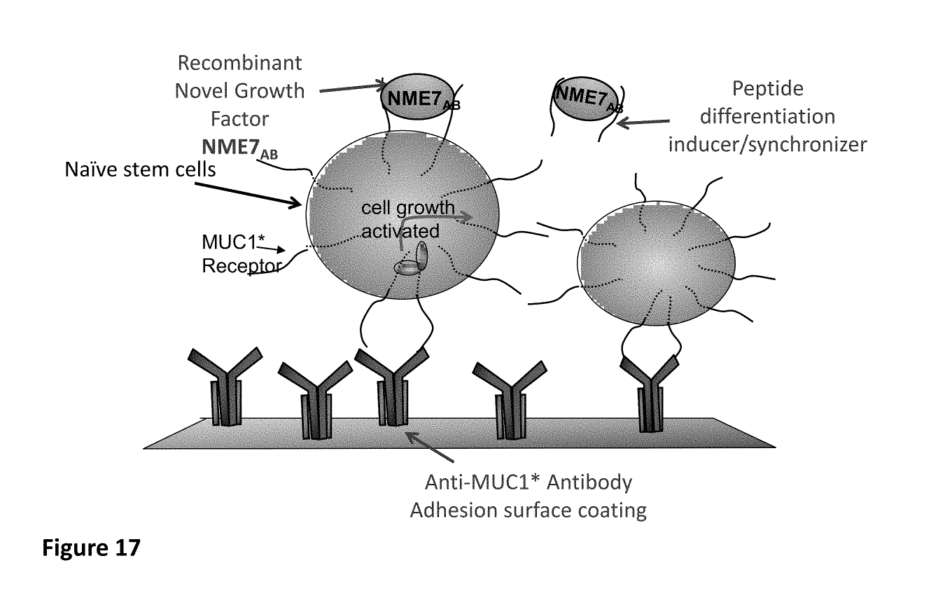

FIG. 17 depicts the inventive method for growing stem cells in the earliest naïve state. NME7-AB or NME7-X1 is added to a serum-free media at low nanomolar concentrations, with the range being between 1 nM-60 nM, with 2 nM-32 nM more preferred, 2 nM-10 nM more preferred and 4 nM most preferred. Feeder cells and extracellular matrix proteins and mixtures contain growth factors and other biological molecules that deliver signals, so it is desirable to avoid their use when inducing or stabilizing naïve state. Preferred are the use of an anti-MUC1* antibody, such as MN-C3, MN-C8 or humanized versions or fragments of MN-C3 or MN-C8, or an NME protein as the surface coating that makes stem cells adhere to the surface. Alternatively, the cells could be cultured in suspension to avoid to need for an adhesive layer. When it is desired to induce differentiation, a peptide comprising most or all of the PSMGFR peptide is added. Adding a peptide having the sequence of most or all of the extracellular domain of MUC1* growth factor receptor acts as a ligand sink and binds up all the NME growth factor so that differentiation is synchronized and more complete. The addition of this peptide also ensures that all the OCT4 positive cells are induced to differentiate which minimizes or eliminates the risk of teratoma formation. These methods are used to produce stem cells for research, drug discovery, therapeutic use, or can be implanted into a fertilized egg, morula, blastocyst, embryo or fetus for the generation of non-human animals that have some human DNA, molecules, cells, tissues or organs. Such molecules, cells, tissues or organs that contain at least some human DNA can then be used for research, drug discovery, drug testing, or administered to a human for the treatment or prevention of a disease or condition. For insertion of human stem cells into a non-human host, the NME7-AB or NME7-X1 should have the human sequence or a sequence at least 80% identical to the native human sequence. However, it may be desirable to create chimera between non-human species. For example, a non-human primate-non-primate chimera could be created to avoid ethical concerns. In that case, the sequence of the NME protein could be human or the sequence of the non-human primate because of the high sequence homology. However, if it is desired to generate chimera of lower order mammals, then the NME7 sequence should be that of the mammal whose stem cells are to be inserted into the host morula or blastocyst. Alternatively, it may be desirable to have human cells and tissues expressed at low percentages or only in certain areas of the host animal. In those cases, it may be desirable to insert stem cells that are not in the earliest naïve state but in a later naïve state. In those cases, NME1 dimers would be used in to culture the stem cells in the methods described above.

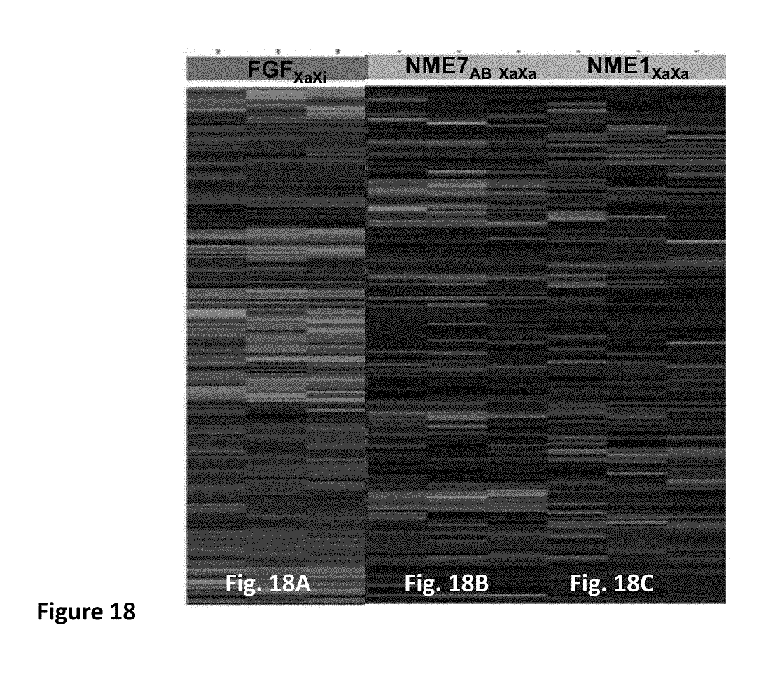

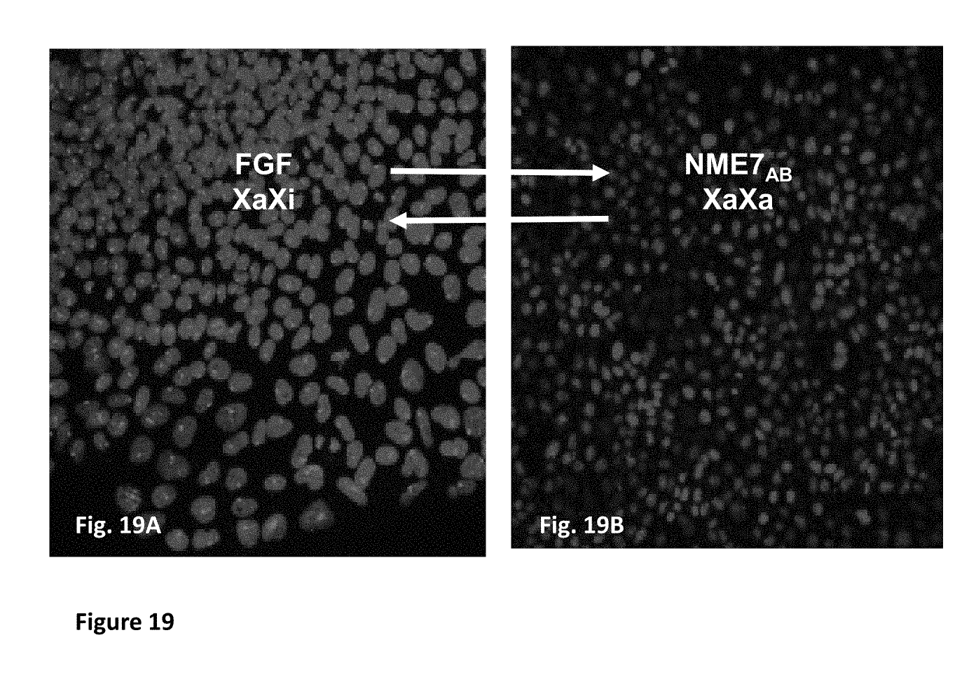

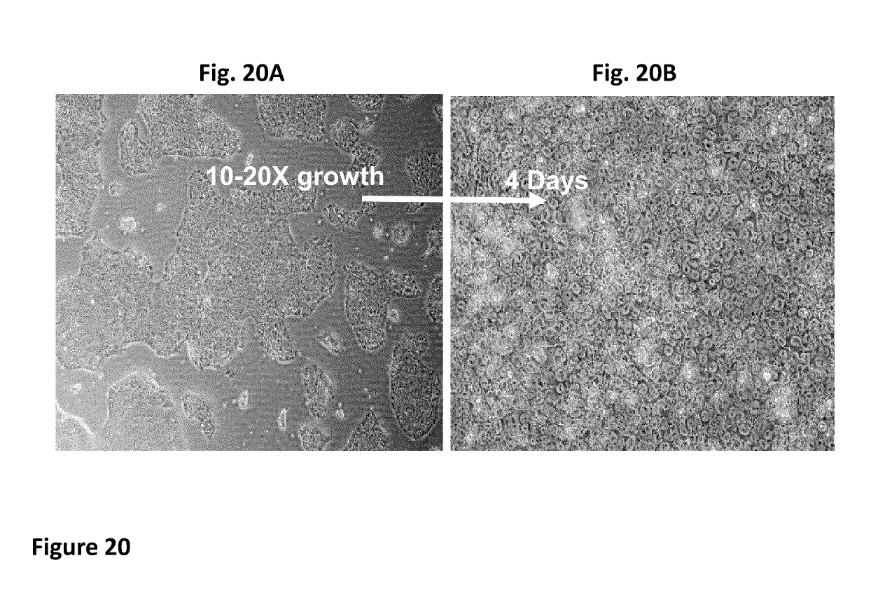

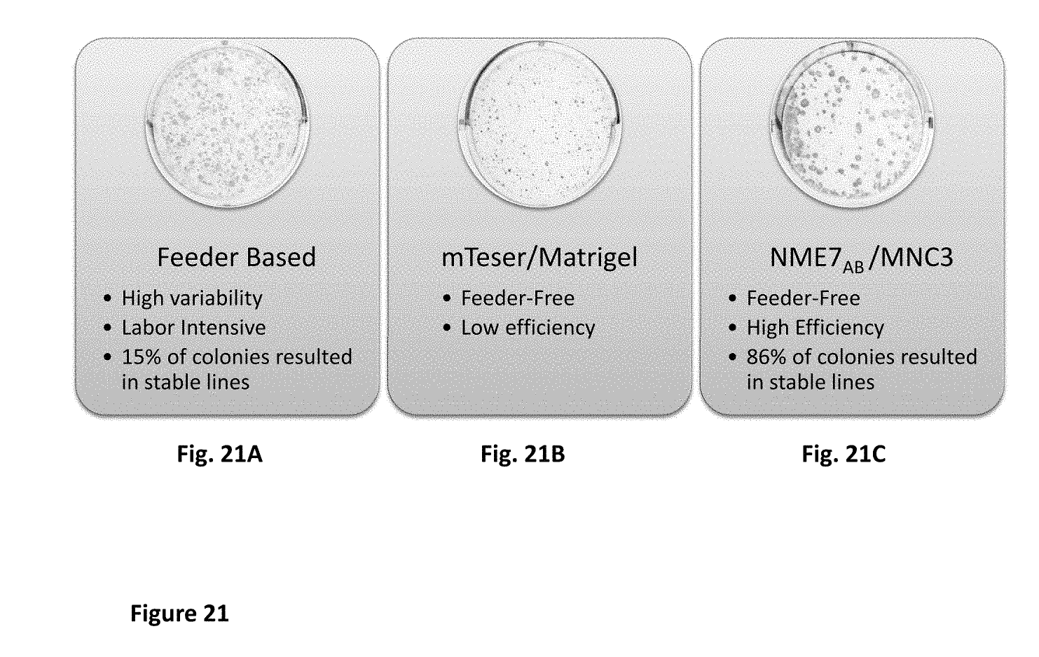

For the generation of a truly chimeric animal, the stem cells to be injected into the fertilized egg, morula, or blastocyst must be in a naïve state. FIG. 18 shows a heat map generated from RNA-SEQ experiments. Human embryonic stem cells that had been derived and grown in FGF so they were in the primed state, were then transferred to the culture system described above wherein the added growth factor was either NME7-AB or NME1 dimers. The heat map of gene expression shows that FGF grown primed state cells have a completely different gene expression signature from that of NME7-AB grown cell or NME1 grown cells, indicating that they are different from primed cells and are naïve. Another indication that NME7 and NME1 grown cells are in the naïve state is that iPS cell generation in NME7-AB, NME1 dimers or NME7-X1 is much more efficient than iPS generation in FGF based media. FIG. 19A-19C shows that reprogramming somatic cells to become induced pluripotent stem cells (iPS cells) in FGF-based media has very low efficiency. Stem cell colonies are visualized by staining with alkaline phosphatase. FGF-based media used with mouse embryonic fibroblasts (MEFs) looks relatively efficient at this early stage but only about 15% of the picked colonies proceed to become bona fide stem cell lines (FIG. 19A). Additionally, the mouse feeder layer introduces non-human non-quantifiable and non-human species into the method which is frowned up for eventual therapeutic use of the cells or their progeny in humans. mTeSR is an FGF-based media that can be used feeder-free by plating cells onto Matrigel but this method is very inefficient as can be seen by the sparse and small colonies of FIG. 19B. In contrast, iPS reprogramming in NME7-AB over a layer of anti-MUC1* antibody is highly efficient with an abundance of stem cell colonies that arise quicker than FGF-based methods and grow faster (FIG. 19C). In addition, over 86% of colonies picked from NME7-generated iPS colonies go on to become bona fide naïve state iPS stem cell lines. Another indicator of stem cells being in the naïve state is if the second X chromosome of female source cells is still active. One of the earliest differentiation decisions that stem cells make is which X chromosome will be turned off in female cells. To turn off expression of one X chromosome, Lysine 27 of histone 3 is tri-methylated. FIG. 20A shows female source embryonic stem cells derived and grown in FGF media. The focal red dot in the nucleus of each cell is a fluorescent antibody binding to the tri-methylated Lysine 27 in histone 3 showing that in primed state stem cells the second X chromosome has been turned off, called XaXi. FIG. 20B shows that after culturing these same cells in NME7-AB, the second X is reactivated (XaXa) resulting in the disappearance of the focal red dot. The same results were obtained after culture in NME1 dimers. A more controversial measure of whether or not stem cells are in the naïve state is if they can incorporate into the inner cell mass of a morula or blastocyst of another species. FIG. 21A-21D shows fluorescent images of our human NME7-AB stem cells incorporating into the inner cell mass of a mouse blastocyst. Yet another indicator of naïve state stem cells is if they grow without spontaneous differentiation and if they grow faster than primed state cells. FIG. 20A-20B shows that human NME7-AB grown stem cells have a much faster growth rate than primed stem cells. They undergo a 10-20-fold expansion in 4 days, which is 2-3-times faster than primed state cells.

NME proteins promote growth and pluripotency of embryonic and iPS cells as well as inducing cells to revert to a stem-like state or a naïve state. In a preferred embodiment the NME family member protein is NME1 or an NME protein having greater than 30%, 35%, 40%, 45%, 50%, 55%, 60%, 65%, 70%, 75%, 80%, 85%, 90%, 95%, or 97% sequence identity to NME1, wherein said protein is a dimer. In a more preferred embodiment, the NME family member protein is NME7 or an NME protein having greater than 30%, 35%, 40%, 45%, 50%, 55%, 60%, 65%, 70%, 75%, 80%, 85%, 90%, 95%, or 97% sequence identity to at least one of the NME7 domains A or B and able to dimerize the MUC1* growth factor receptor.

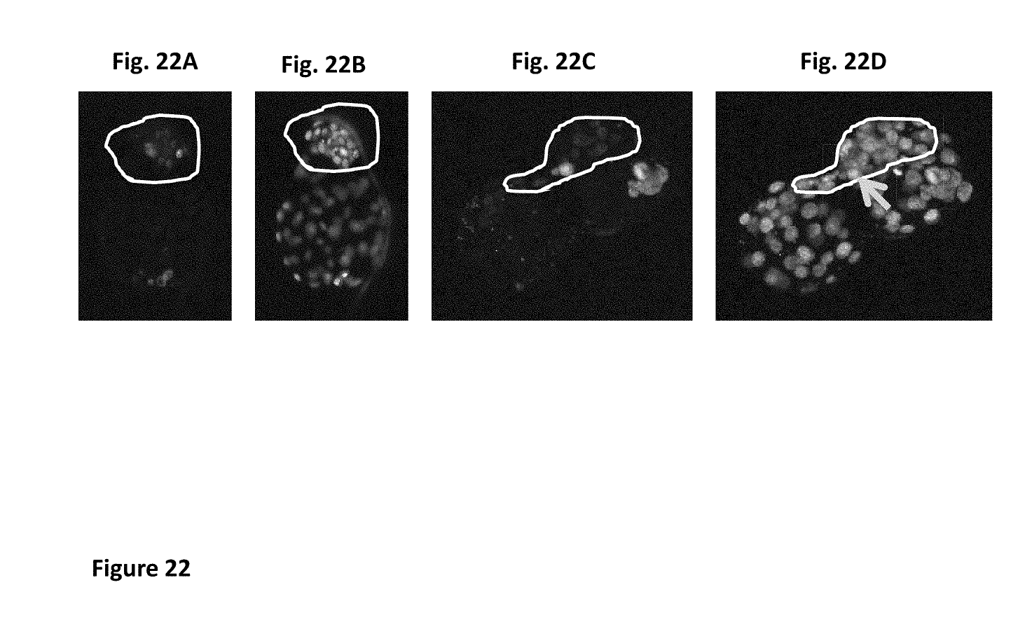

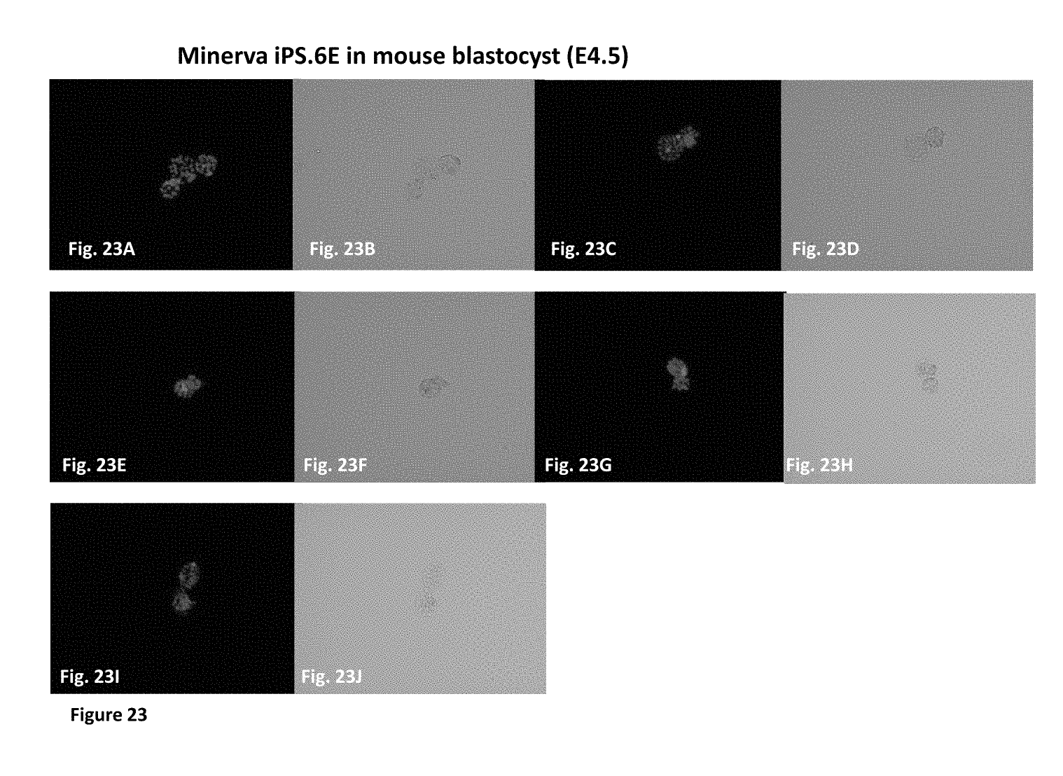

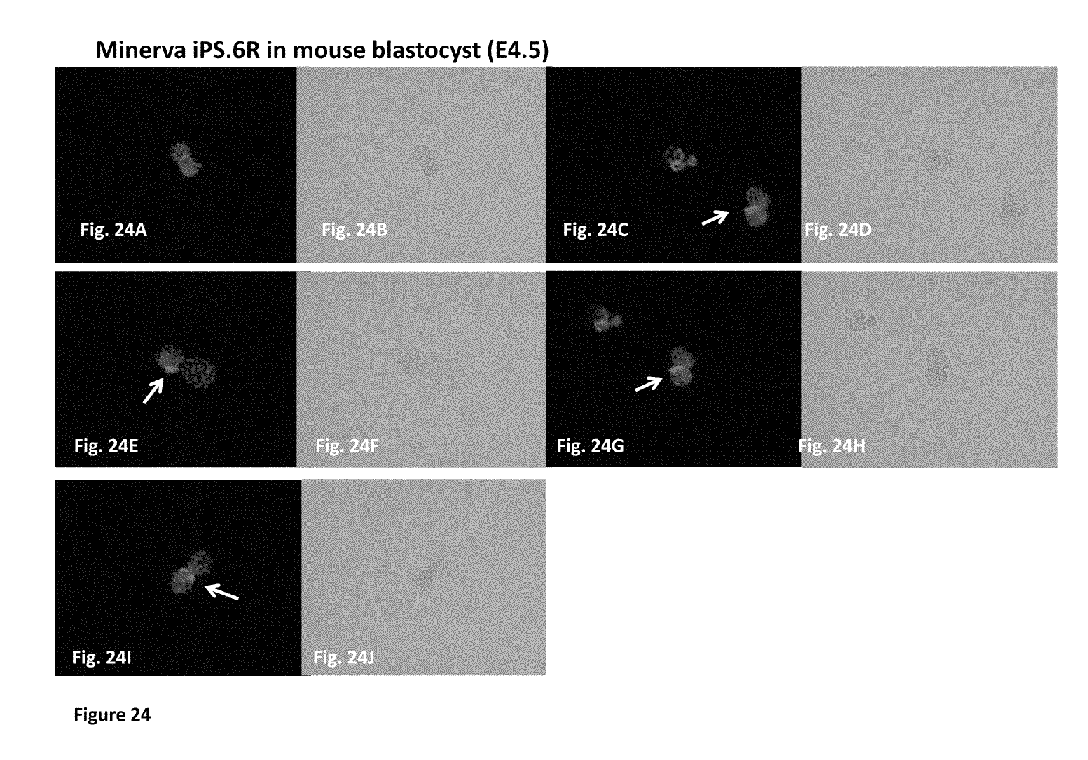

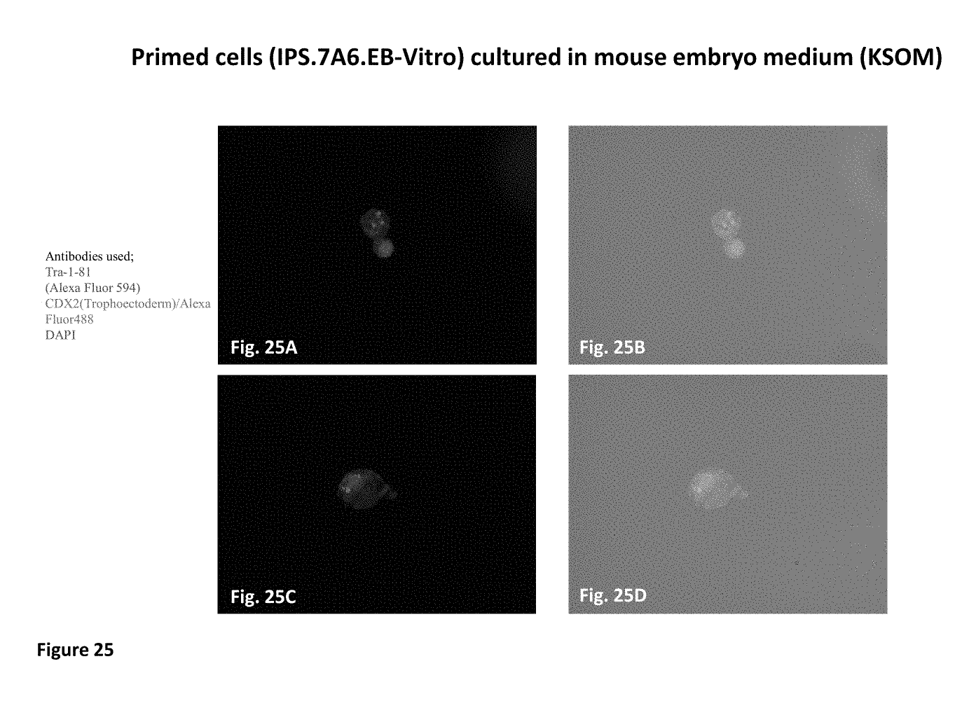

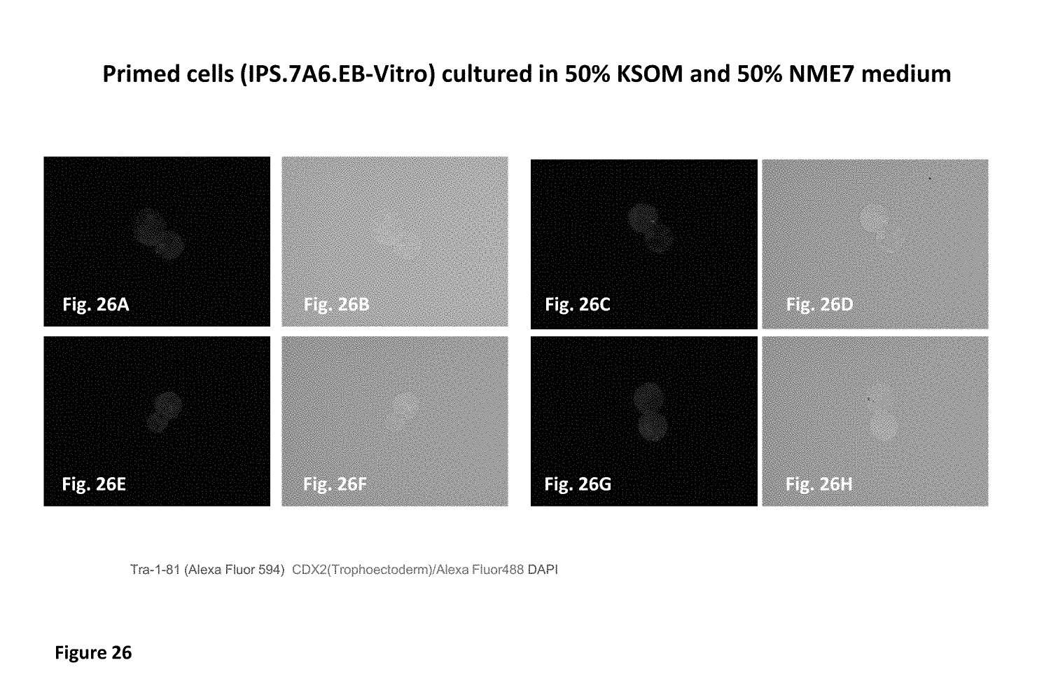

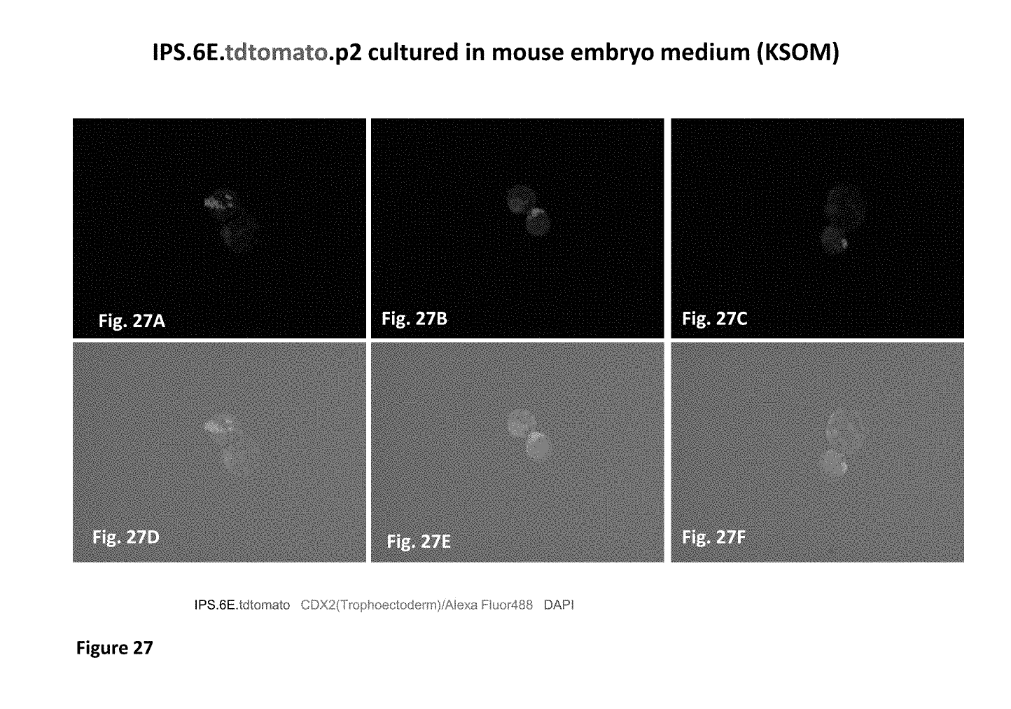

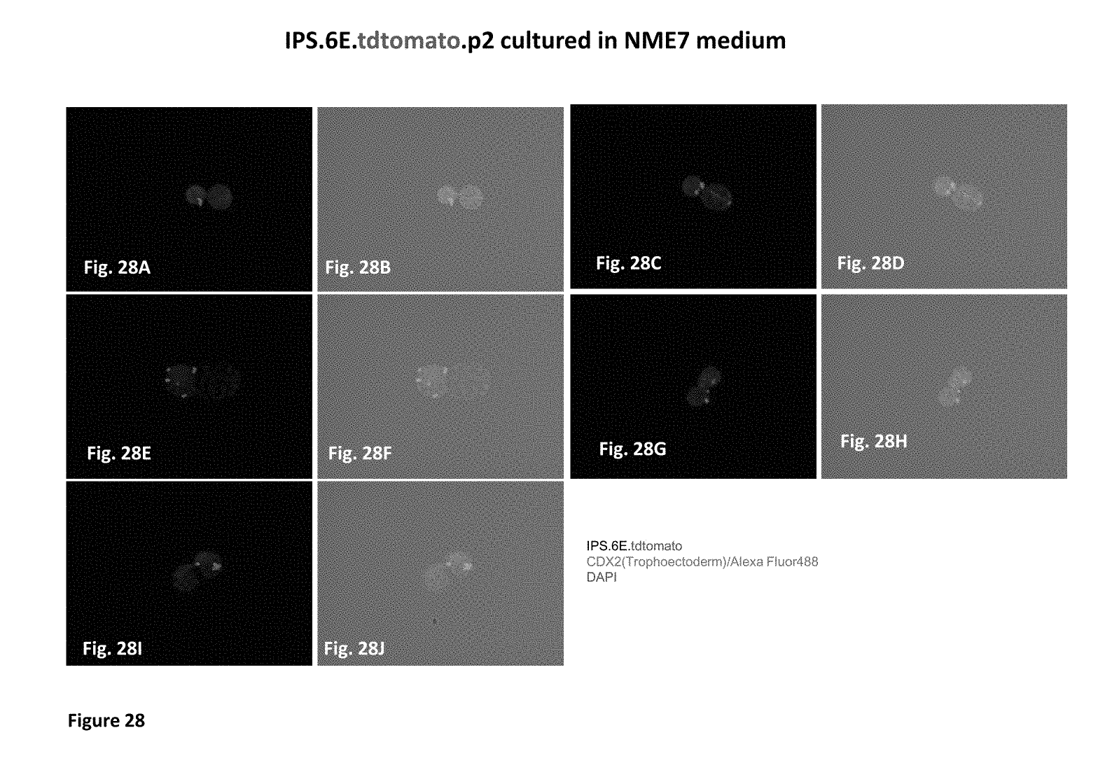

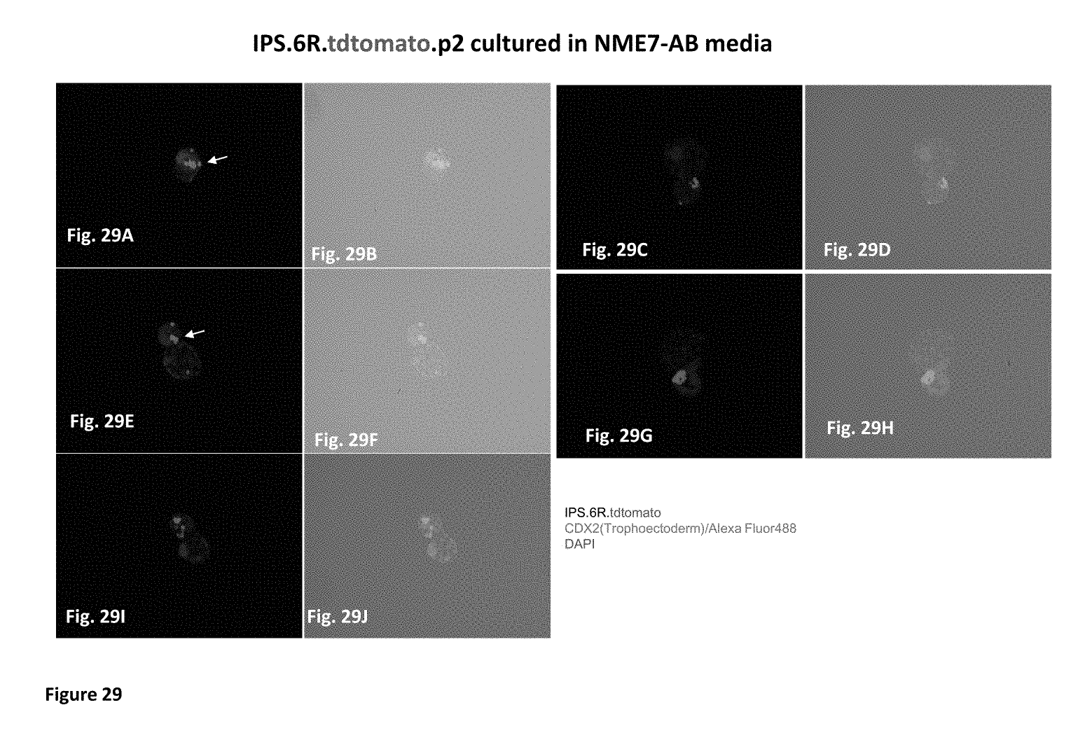

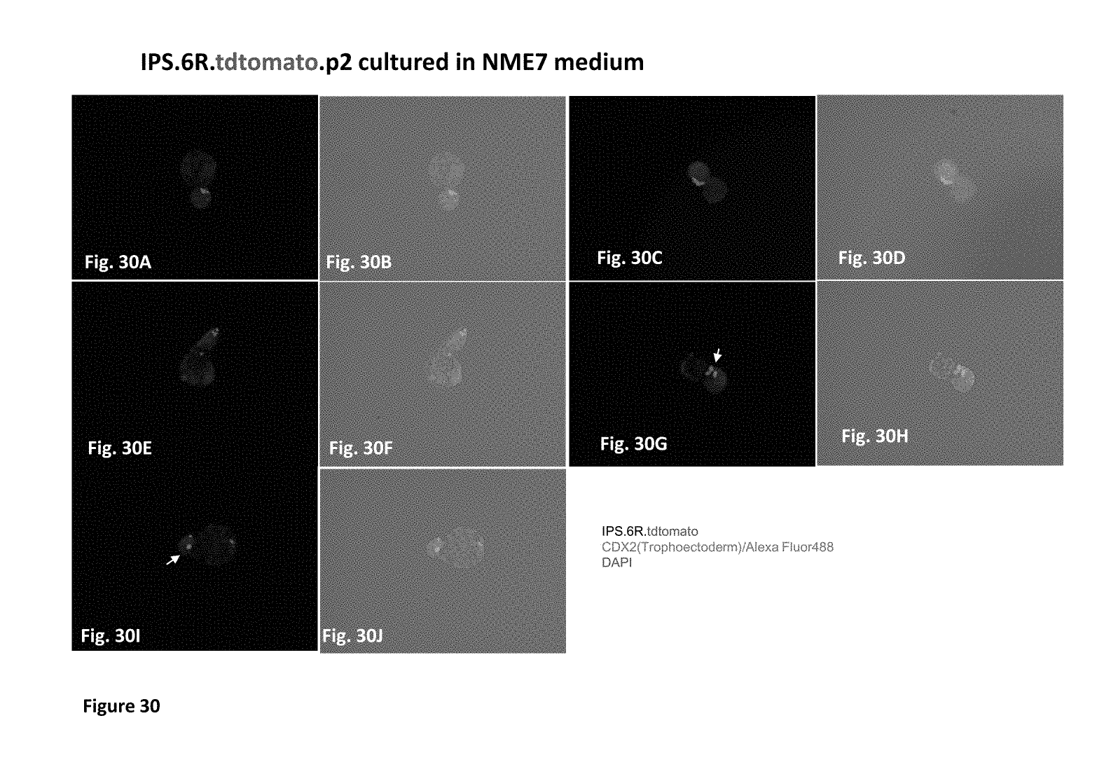

Here, we report that NME1 in dimer form, NME7-AB or NME7-X1 were able to: a) fully support human ES or iPS growth and pluripotency, while inhibiting differentiation; b) revert somatic cells to a more stem-like or naive-like state; and c) produce naïve state human stem cells that were able to integrate into blastocysts of other species. NME7-AB naïve human stem cells were injected into a mouse morula at Day 2.5. FIG. 22A-22D shows that 48 hours later when the morula has developed into a blastocyst, the human cells (yellow) are within the inner cell mass (circled) where the mouse naïve cells reside. Such incorporation into the inner cell mass is required for formation of a chimeric animal. FIGS. 23A-23J and FIGS. 24A-24J show confocal images of other mouse blastocysts into which human NME7-naïve cells were injected at Day 2.5 and also show integration into inner cell mass. FIGS. 25A-25D and FIG. 26A-26H show that primed state, FGF grown human stem cells do not incorporate into the inner cell mass of mouse blastocysts under the same conditions. FIGS. 27A-27F, FIGS. 28A-26J, FIGS. 29A-29J, and FIGS. 30A-30J show human NME7-AB grown naïve stem cells injected at Day 2.5 into a mouse morula, are found concentrated in the inner cell mass of the blastocyst 48 hours later at Day 4.5.

We made recombinant human NME1, dimers that bear the 5120G mutation that stabilizes dimers. We previously reported that human NME1 dimers bind to the PSMGFR portion of the extracellular domain of the MUC1* receptor (Smagghe et al. 2013). We also made recombinant human NME7-AB and NME7-X1 that, as monomers, bind to and dimerize the PSMGFR portion of the extracellular domain of the MUC1* receptor on pluripotent stem cells, which stimulates growth and pluripotency and induces stem cells to revert to the earliest naïve state.

NME is a Universal Stem Cell Growth Factor

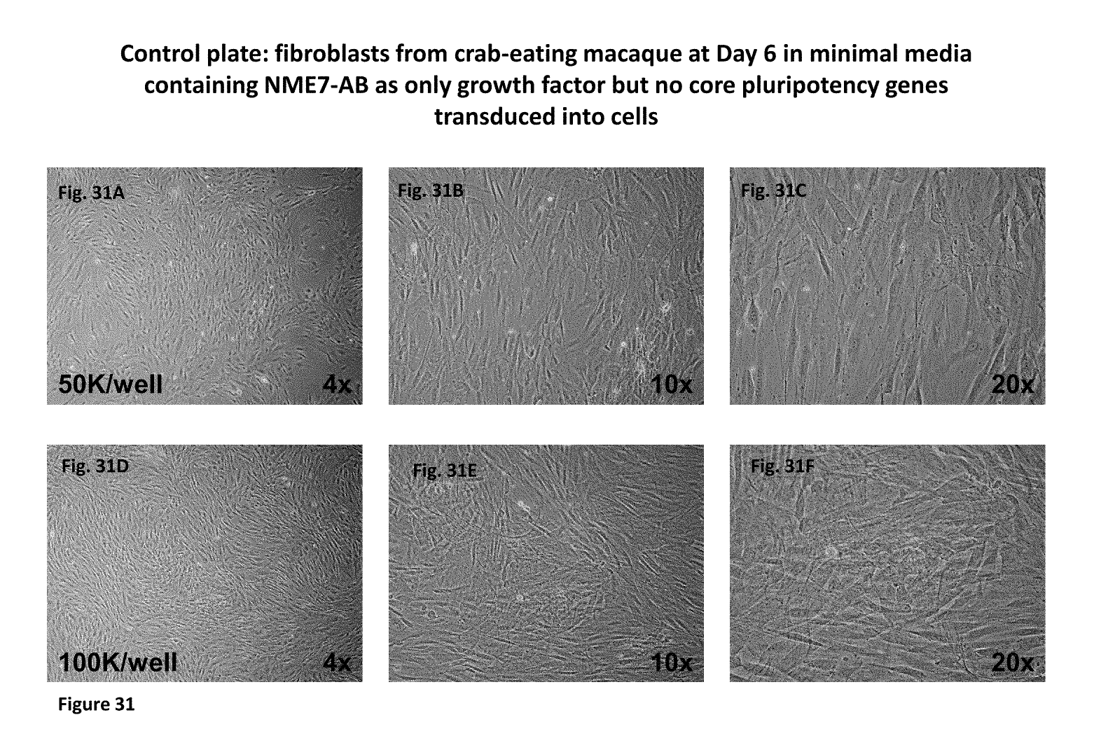

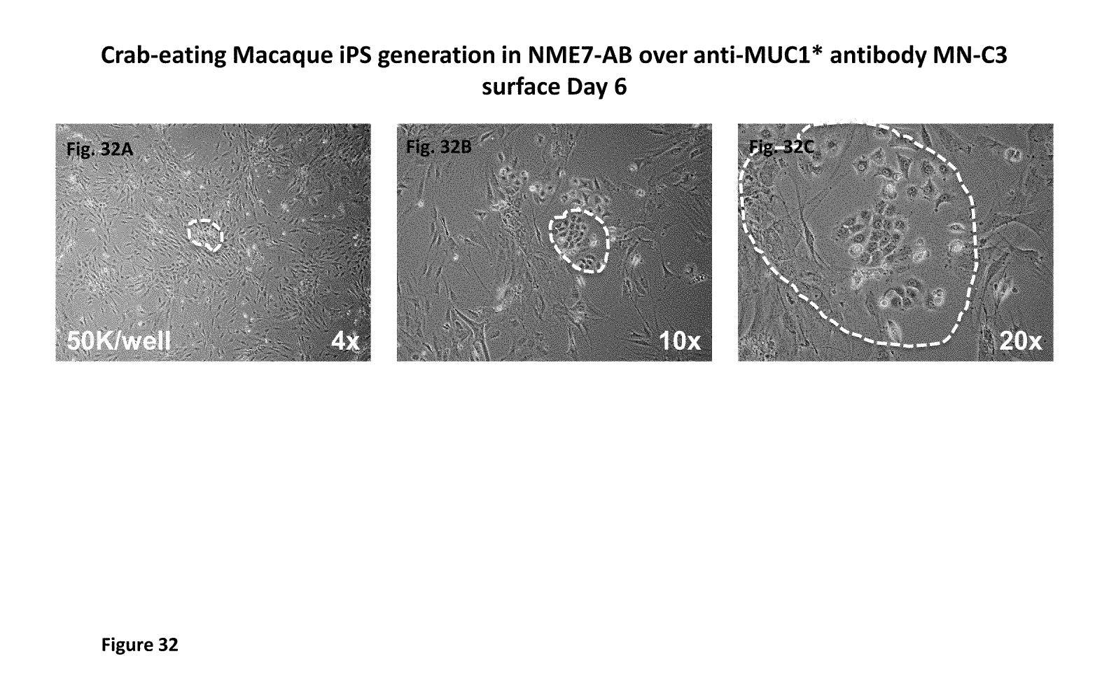

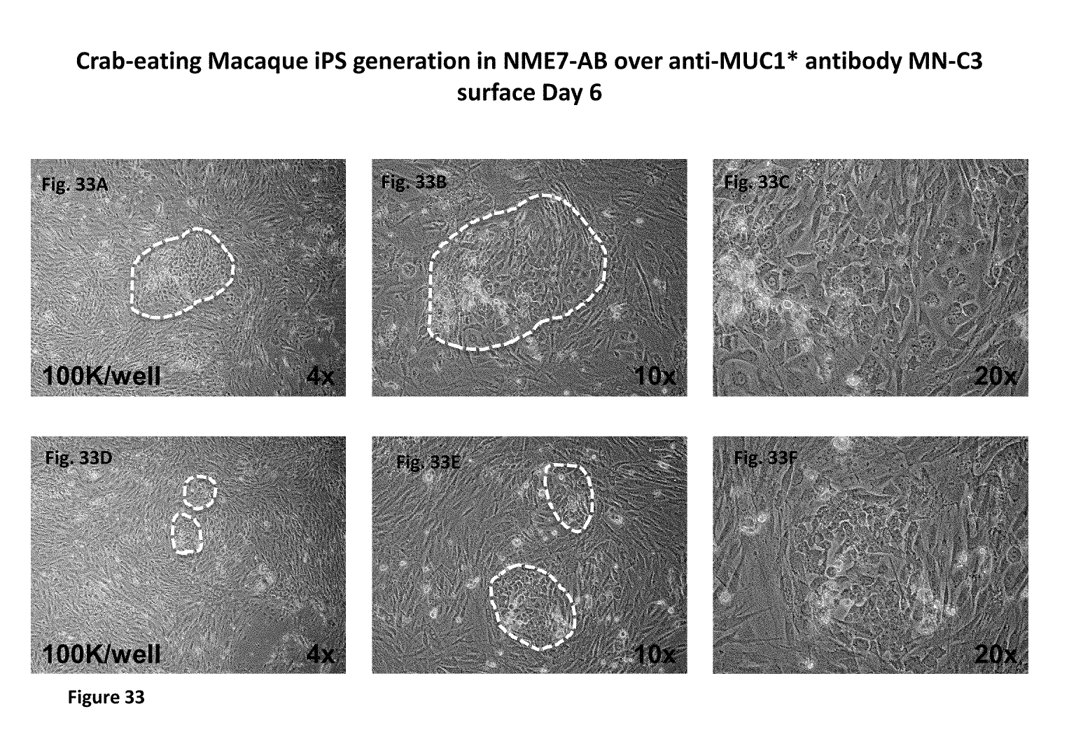

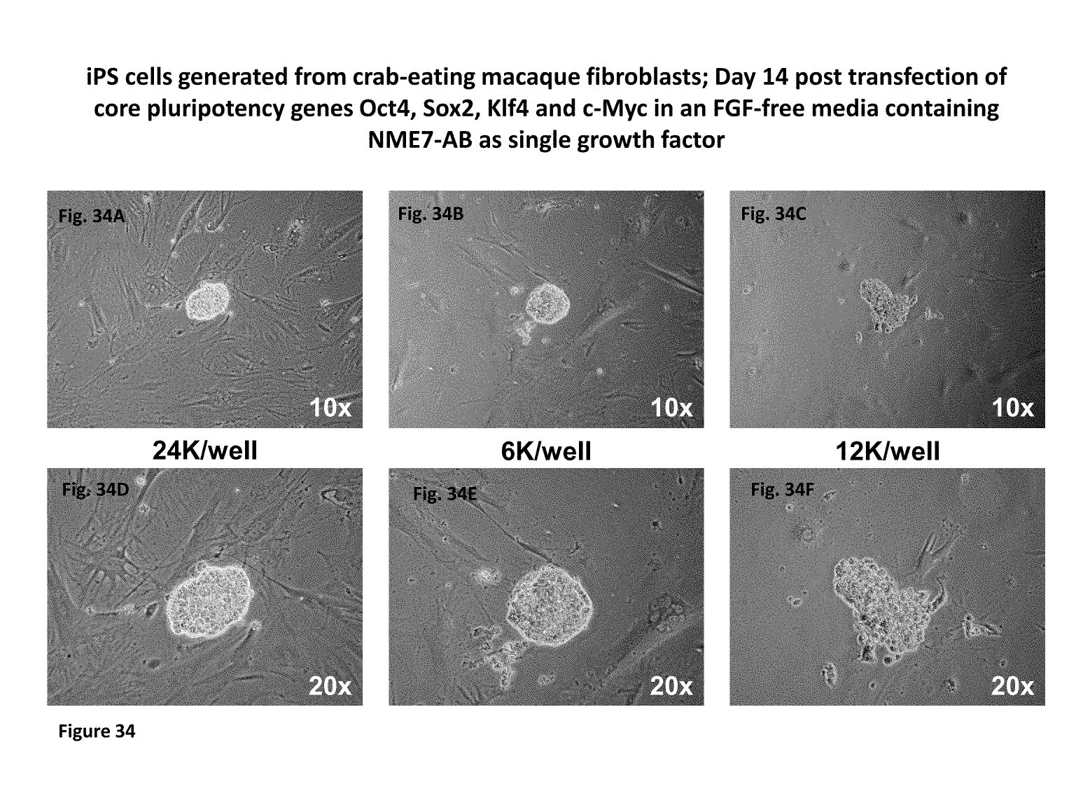

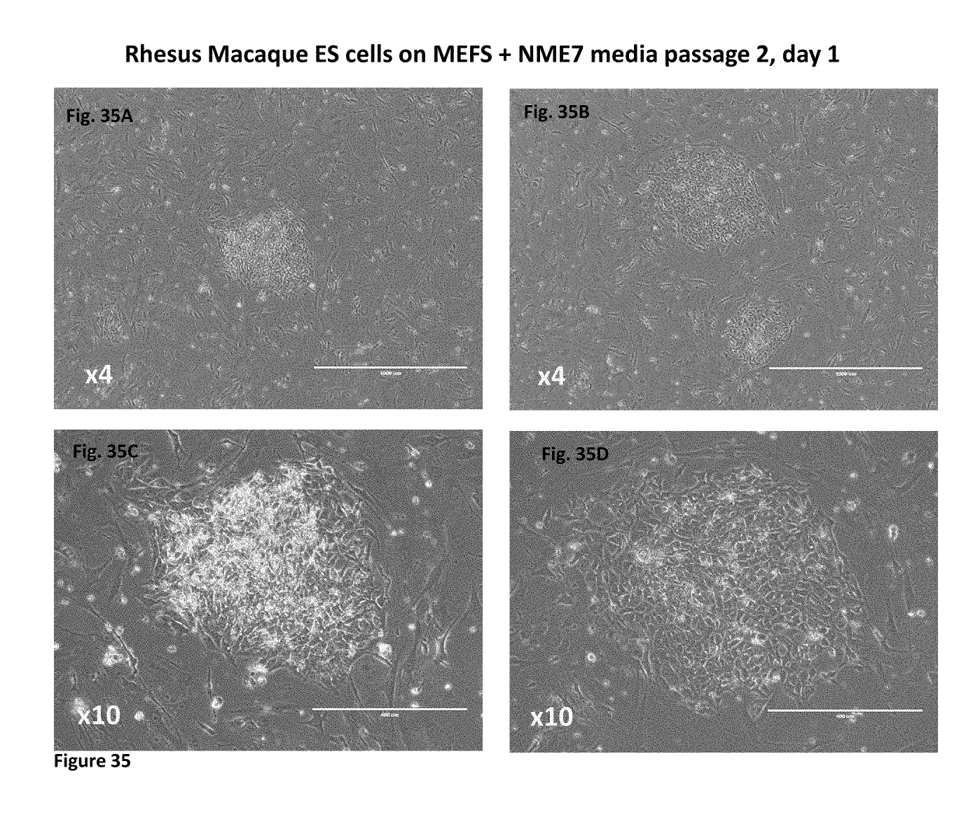

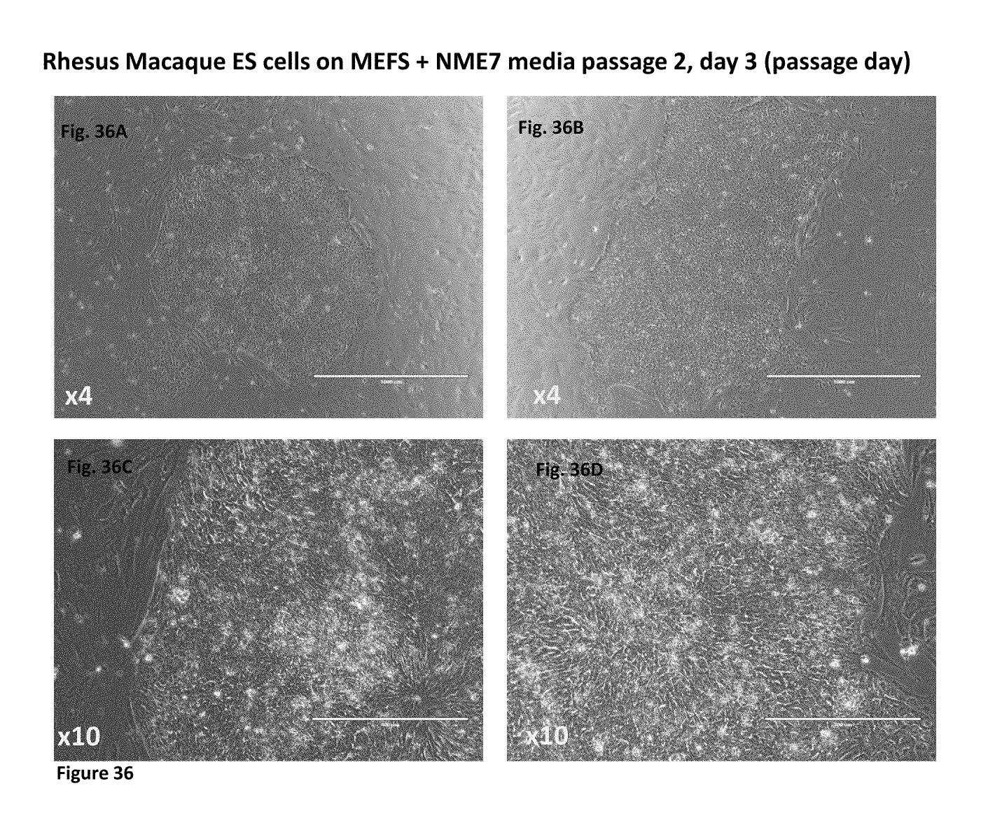

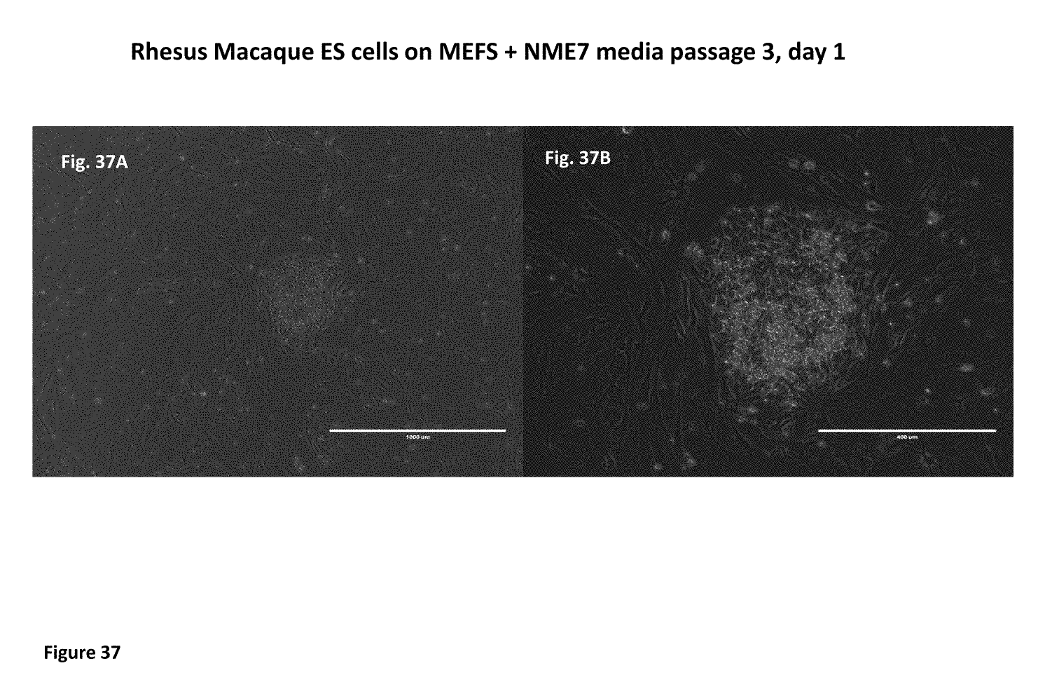

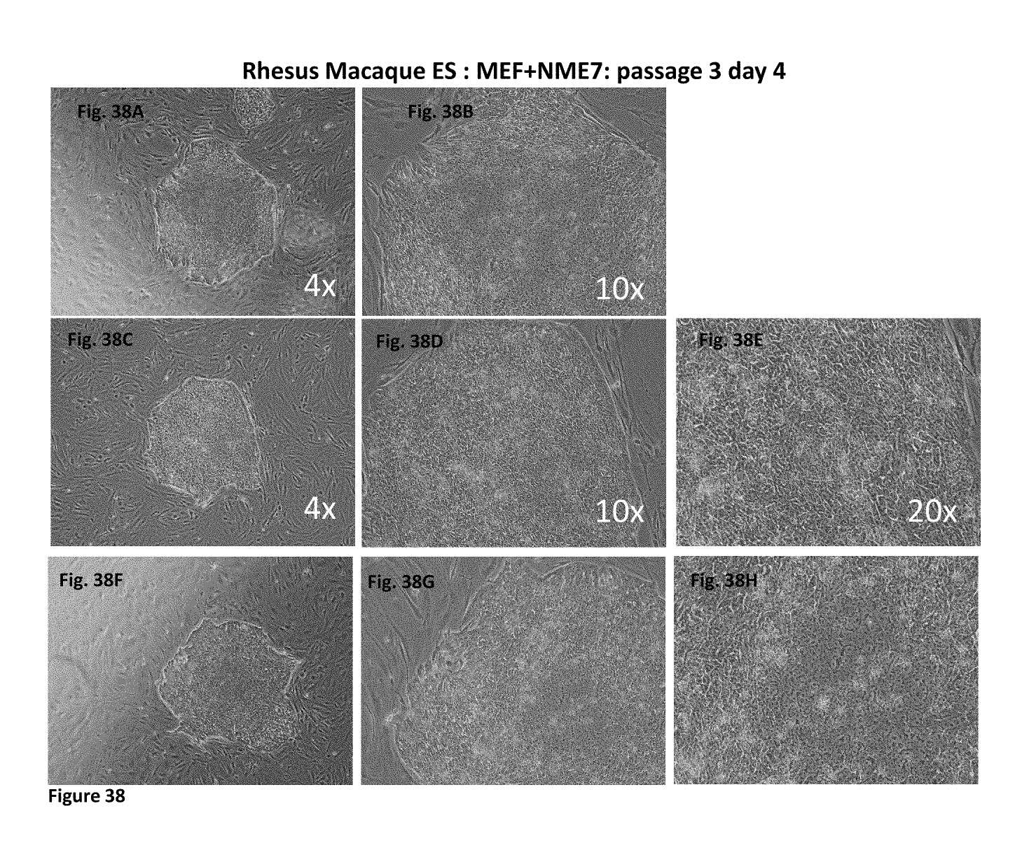

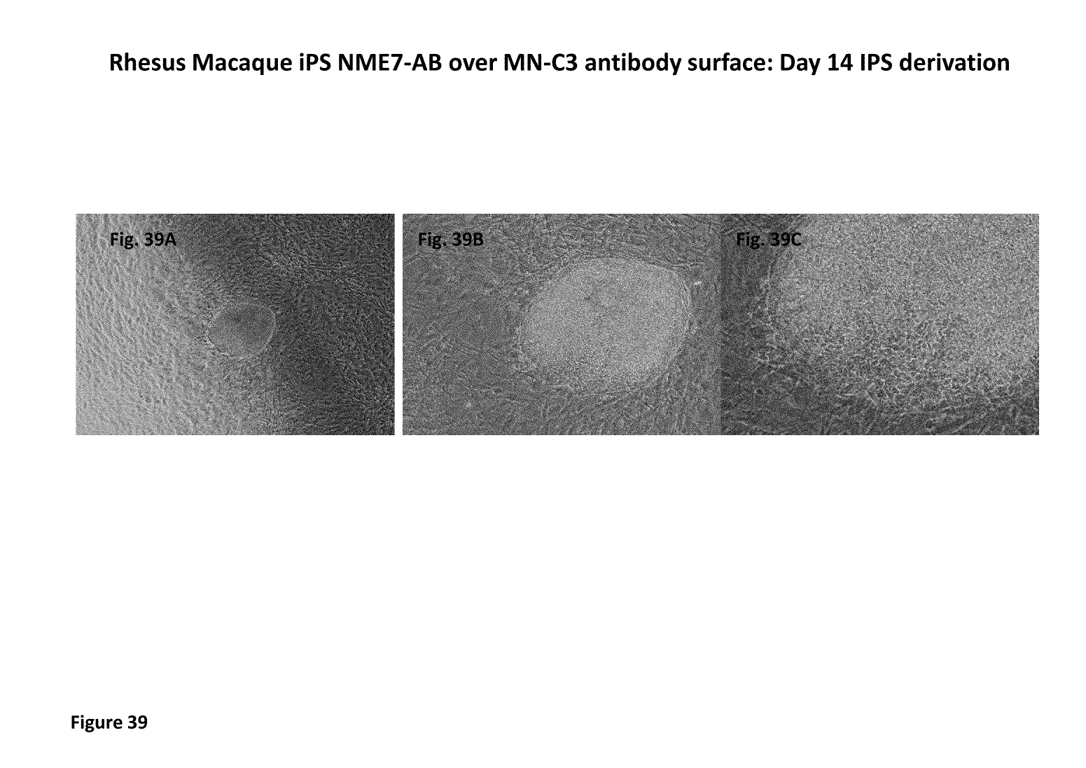

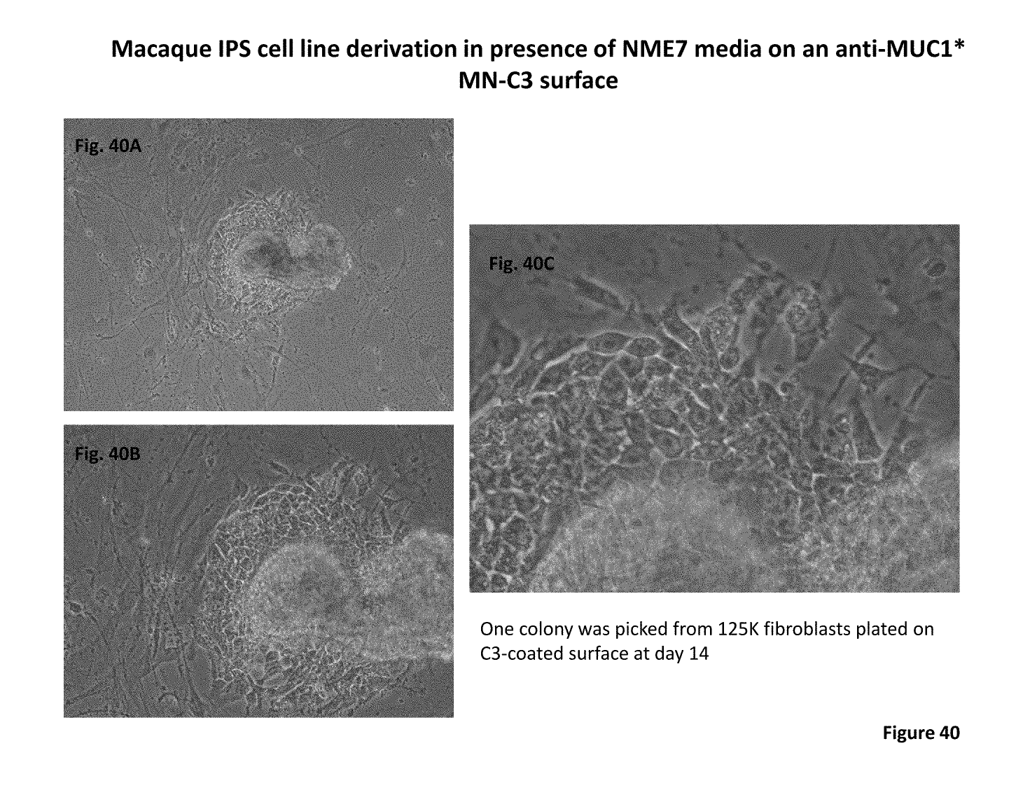

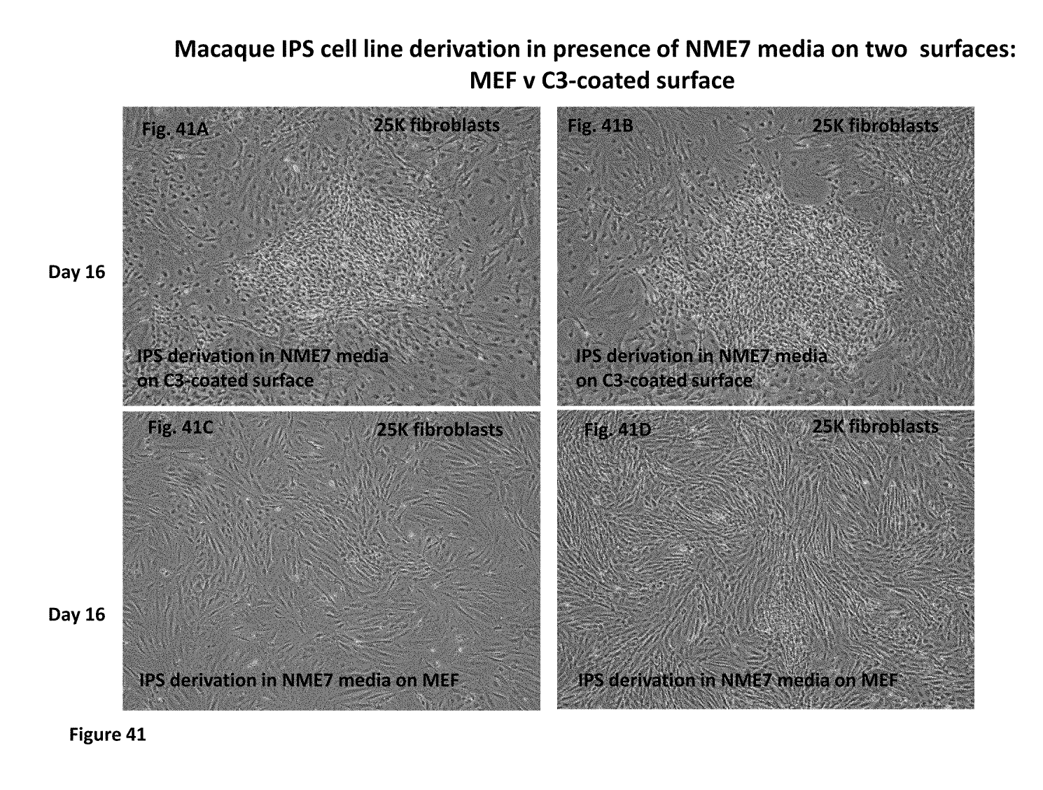

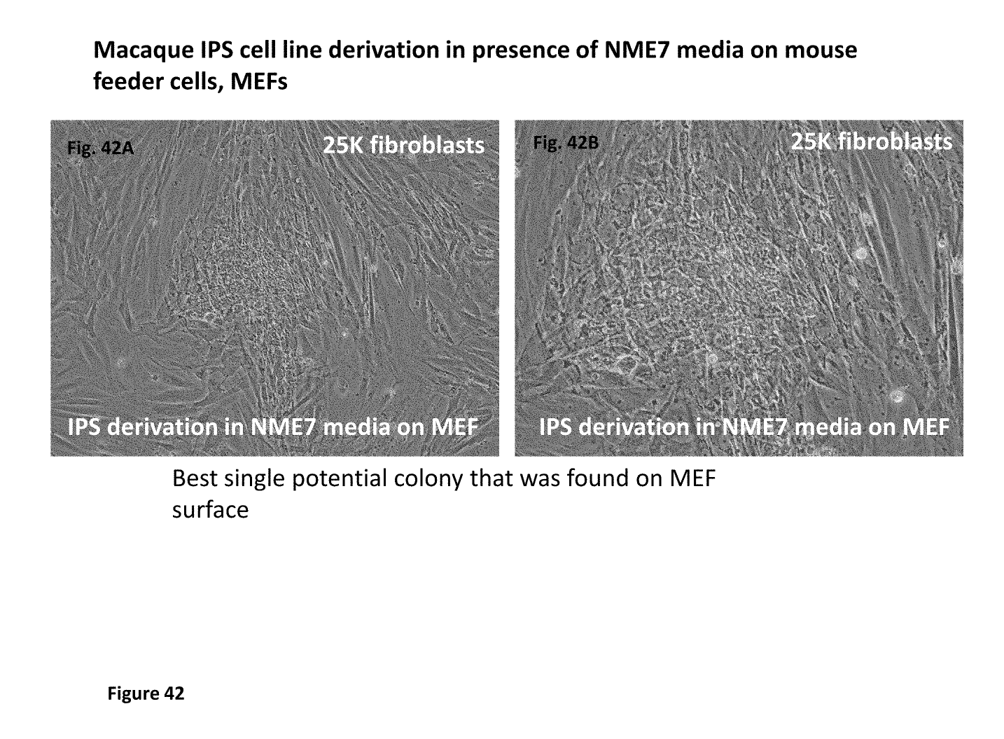

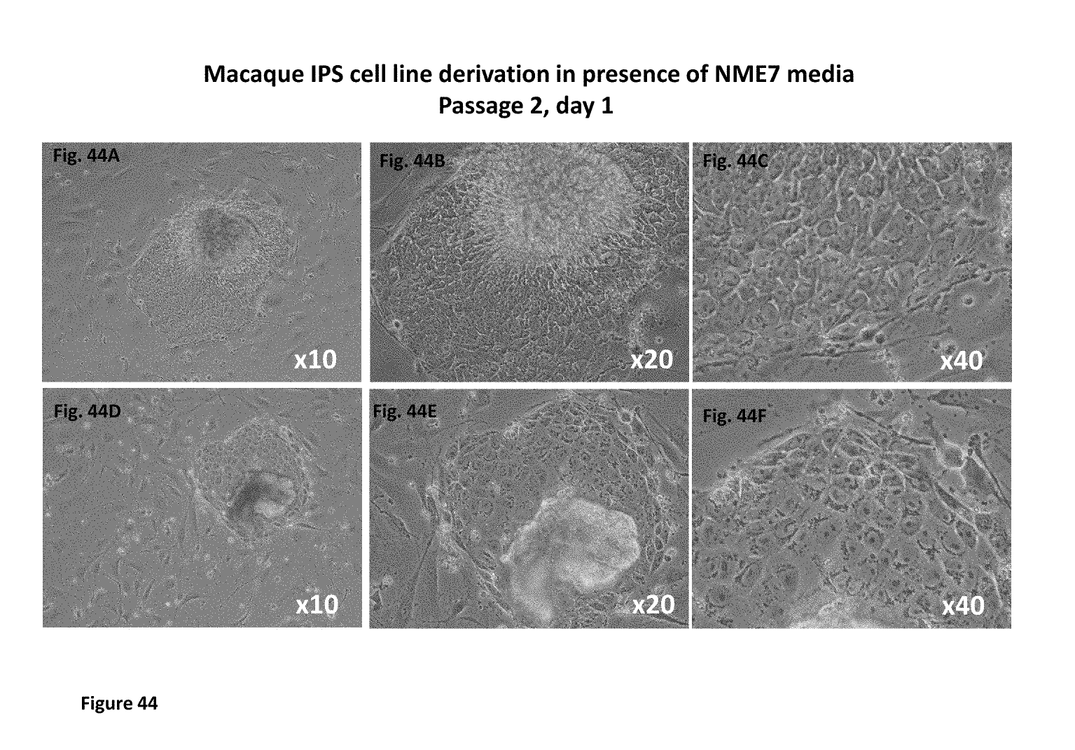

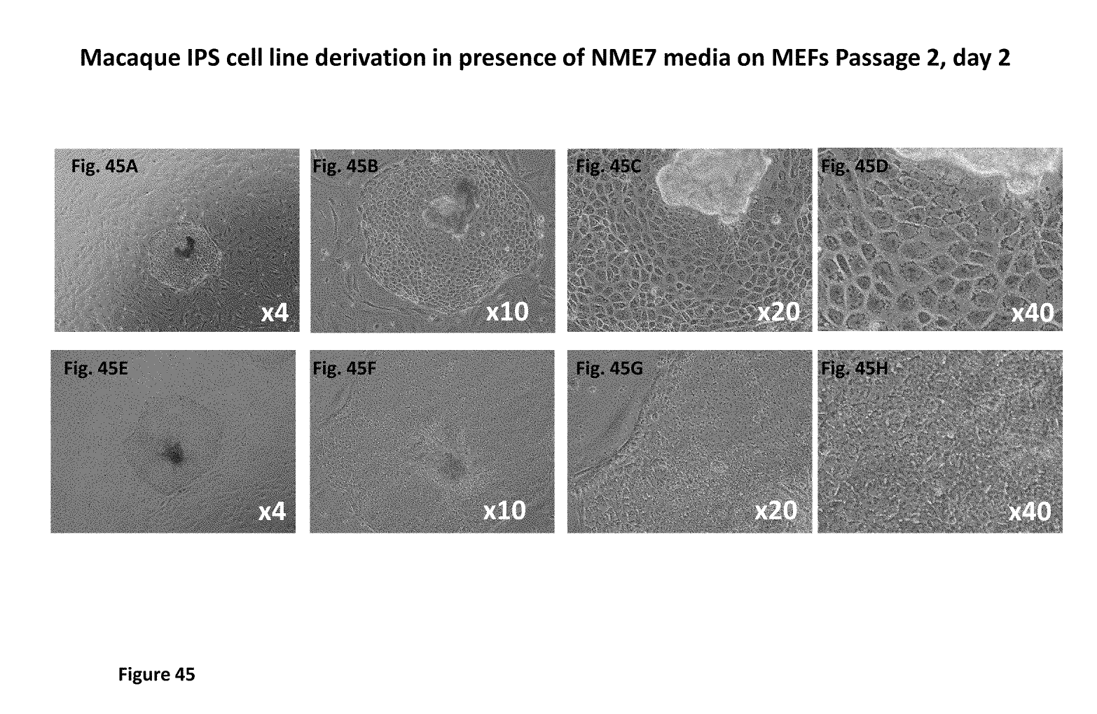

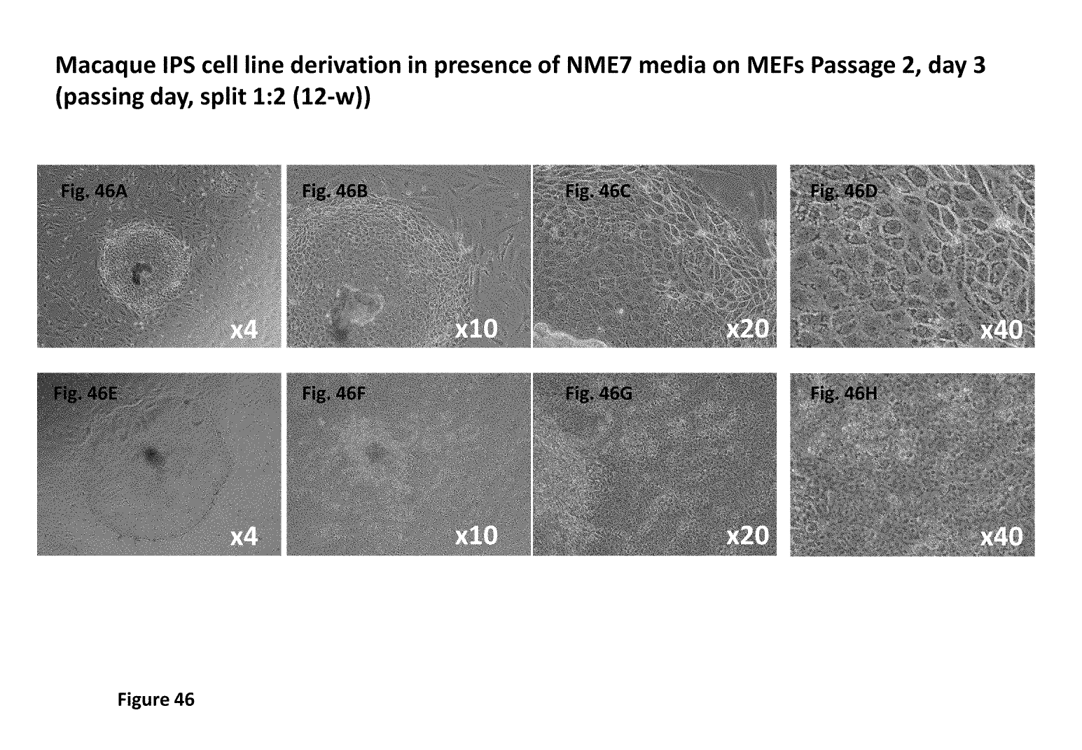

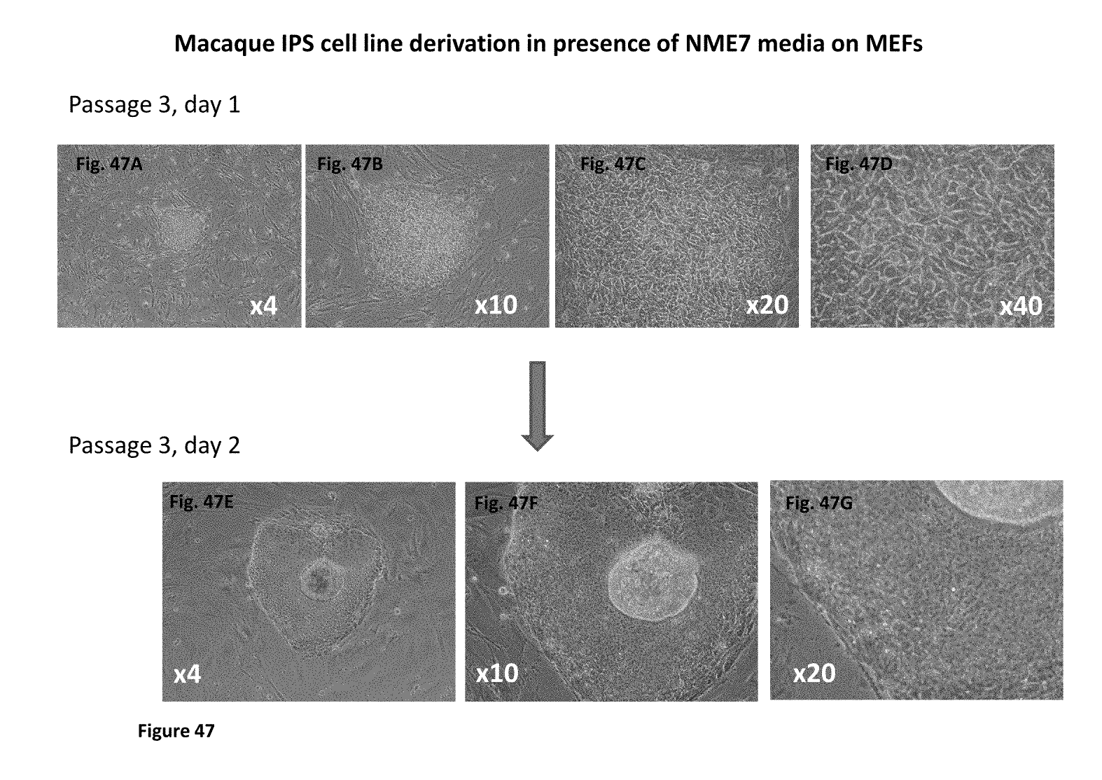

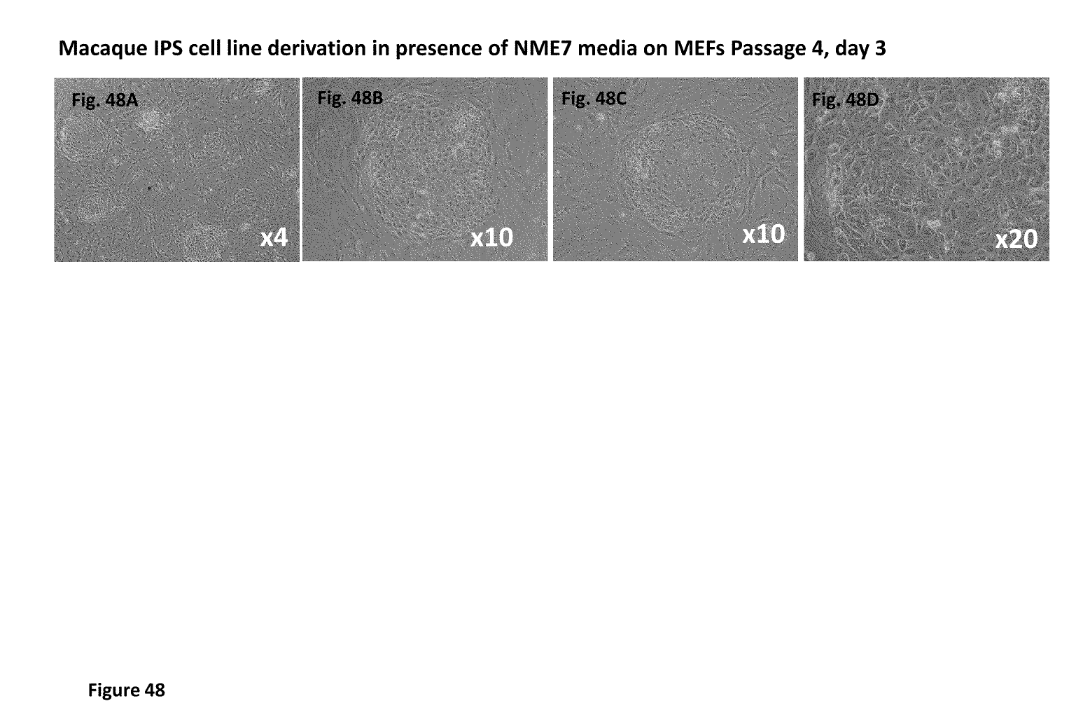

NME7 and truncated forms are universal stem cell and pluripotency growth factors across many species. For example, we have been able to proliferate rhesus macaque and crab-eating macaque stem cells, including embryonic and iPS stem cells using human NME7-AB. We have also succeeded in generating iPS cells in rhesus and crab-eating macaque using NME7-AB along with reprogramming factors of Yamanaka or Thomson, in the absence of feeder cells using an anti-MUC1* antibody, MN-C3, to facilitate surface attachment. The sequence of both macaque NME7 is 98% identical to human NME7 and its target growth factor receptor, MUC1* extracellular domain is 90% identical to human PSMGFR. FIGS. 31A-31F show photograph of control plates containing fibroblasts from crab-eating macaques in culture in NME7-AB for 6 days but without transduction of Yamanaka pluripotency factors OCT4, SOX2, NANOG and c-Myc. FIGS. 32A-32C, FIGS. 33A-33F, and FIGS. 34A-34F show fibroblasts from crab-eating macaques being reprogrammed by Yamanaka factors and NME7-AB. After Day6, emerging iPS colonies are transferred to an anti-MUC1* antibody surface, where they continue to expand until about Day 14-17 when individual clones are picked and expanded. To our knowledge, this is the first time scientists succeeded in generating non-human primate iPS cells in the absence of mouse or human feeder cells. Researchers experience extreme difficulty culturing non-human primate stem cells. They differentiate spontaneously and do not grow well in FGF-based media. We overcame all these problems when we moved non-human primate cells into NME7-AB media, shown in FIGS. 35A-35D, FIGS. 36A-36D, FIGS. 37A-37B, and FIGS. 38A-38H. Generation of rhesus macaque iPS cells by transduction of Yamanaka factors or Thomson factors in serum-free, FGF-free, NME7-AB media over MN-C3 antibody surface or over MEFs is shown at various stages in FIGS. 39A-39C, FIGS. 40A-40C, FIGS. 41A-41D, FIGS. 42A-42B, FIGS. 43A-43E, FIGS. 44A-44F, FIGS. 45A-45H, FIGS. 46A-46H, FIGS. 47A-47G, and FIGS. 48A-48D.

In another aspect of the invention, embryonic or iPS stem cells are proliferated, maintained or generated in other non-human species by culturing fertilized eggs or embryos or reprogramming cells using a media that contains an NME protein. The NME protein may be NME1 dimers or NME6 dimers, NME7, NME7-AB or NME7-X1. In some cases, the sequence of the NME protein may be the human sequence. In other cases the sequence of the NME protein is the sequence of the non-human species that the stem cells, embryo or cells for reprogramming came from. If the sequence of NME7 in the non-human species is too diverse from that of human NME7, then better results are obtained using an NME protein having a sequence that has higher sequence identity to the non-human target species or an NME protein having the sequence of the native NME protein of that species. In one aspect of the invention the NME protein is NME1 or NME6 dimers, NME7, NME7-AB or NME7-X1. A method for determining which species a particular NME protein will work in as a pluripotency growth factor is to test whether or not the NME protein binds to a PSMGFR peptide having the sequence of the target species. If the NME protein binds to the PSMGFR peptide of the target species, then the NME protein will work as a stem cell growth factor or pluripotency factor.

In one aspect of the invention, non-human stem cells are proliferated or maintained by culturing the cells in a media containing an NME protein of that non-human species. In another aspect of the invention, non-human stem cells are proliferated or maintained by culturing the cells in a media containing an NME protein having a sequence that is at least 40% homologous to that of the non-human species stem cells. In another aspect of the invention, non-human stem cells are proliferated or maintained by culturing the cells in a media containing an NME protein that is able to bind to a peptide having the sequence of that species' MUC1* extracellular domain also called the PSMGFR peptide. In another aspect of the invention, the non-human stem cells are proliferated or maintained by culturing the cells in a media containing a human sequence NME protein. The NME protein may be NME1 or NME6 dimers, NME7, NME7-AB or NME7-X1.

In another aspect of the invention, non-human stem cells are generated using reprogramming technology such as introducing combinations of the genes or gene products that include Oct4, Sox2, Klf4, c-Myc, Nanog, Lin28, in the presence of a media containing an NME protein of that non-human species. In another aspect of the invention, non-human stem cells are generated using reprogramming technology in a media containing an NME protein having a sequence that is at least 40% homologous to that of the non-human species stem cells. In another aspect of the invention, non-human stem cells are generated using reprogramming technology in a media containing an NME protein that is able to bind to a peptide having the sequence of that species' MUC1* extracellular domain also called the PSMGFR peptide. In another aspect of the invention, the non-human stem cells are generated using reprogramming technology in a media containing a human sequence NME protein. The NME protein may be NME1 or NME6 dimers, NME7, NME7-AB or NME7-X1.

In another aspect of the invention, non-human embryonic stem cells are generated by culturing fertilized eggs or embryos in a media containing an NME protein of that non-human species. In another aspect of the invention, non-human embryonic stem cells are generated by culturing the fertilized eggs or embryos in a media containing an NME protein having a sequence that is at least 40% homologous to that of the non-human species stem cells. In another aspect of the invention, non-human embryonic stem cells are generated by culturing fertilized eggs or embryos in a media containing an NME protein that is able to bind to a peptide having the sequence of that species' MUC1* extracellular domain also called the PSMGFR peptide. In another aspect of the invention, the non-human embryonic stem cells are generated by culturing fertilized eggs or embryos in a media containing a human sequence NME protein. The NME protein may be NME1 dimers, or NME6 dimers, NME7, NME7-AB or NME7-X1.

In another aspect of the invention, when it is desired to have human stem cells incorporate into the developing blastocyst or embryo of a non-human species whose NME protein or MUC1* protein have low sequence identity to human, the non-human species is humanized. The fertilized or unfertilized egg, or stem cells of the non-human species are transduced with vectors that enable the expression of human NME protein and or human MUC1* protein, wherein their expression may be inducible or repressible.

Testing for Potential Drug Agent in Chimeric Animal

The current practice for testing cancer drugs in mice and other animals is to inject human cancer cells into the animal and either immediately or after several days or weeks of engraftment, inject the animal with the test drug. However, this approach is fundamentally flawed because the host does not naturally produce the growth factors that the human cancer cells need to grow or engraft. Additionally, because the host does not produce the growth factor or the same levels of the growth factor or the human form of the growth factor, drugs being tested in the animals will not have the same effect as they would in humans. Mouse NME7 is only 84% homologous to human NME7 and is not expressed in the adult. Therefore, current xenograft methods for anti-cancer drug testing often fall short in predicting human response to those drugs. This problem could be solved by introducing NME1 dimers or NME7 into the mice so that the human tumor cells have their cognate growth factor to feed the tumor. NME1 dimers or NME7 can be introduced into an animal by a variety of methods. It can be mixed in with the tumor cells prior to implantation, or it can be injected into the animal bearing the tumor.

In a preferred embodiment, a transgenic animal is generated that expresses human NME7 or a fragment thereof. The NME7 may be carried on an inducible promoter so that the animal can develop naturally, but expression of the NME7 or the NME7 fragment can be turned on during implantation of human tumors or for the evaluation of drug efficacies or toxicities. In a preferred embodiment, the NME7 species that is introduced to the test animal is NME7-AB.

Alternatively, a transgenic animal can be made wherein the animal expresses human MUC1, MUC1*, NME7 and/or NME1 or NME2, a variant of NME1 or NME2 that prefers dimer formation, single chain constructs or other variants that form dimers. Because NME proteins and MUC1 are parts of a feedback loop in humans, wherein expression of one can cause upregulation of the other, it could be advantageous to generate transgenic animals that express human NME protein(s) and MUC1 or the cleaved form MUC1*. A natural or an engineered NME species can be introduced into animals, such as mice, by any of a variety of methods, including generating a transgenic animal, injecting the animal with natural or recombinant NME protein or a variant of an NME protein, wherein NME1, NME6 and NME7 proteins or variants are preferred and NME1, NME6 and NME7 proteins or variants that are able to dimerize MUC1*, specifically the PSMGFR peptide, are especially preferred. In a preferred embodiment, the NME species is a truncated form of NME7 having an approximate molecular weight of 33 kDa. In a more preferred embodiment, the NME7 species is devoid of the DM10 domain of its N-terminus. In a still more preferred embodiment, the NME7 species is human.

NME family proteins, especially NME1, NME6 and NME7 are expressed in human cancers where they function as growth factors that promote the growth and metastasis of human cancers. Therefore, human NME protein or active forms of NME protein should be present for proper growth, evolution and evaluation of human cancers and for determining their response to compounds, biologicals or drugs.

Humanized Animals

In some instances, it is desirable to be able to control the timing of the expression of the NME protein. In these cases, the protein expression may be linked to an inducible genetic element such as a regulatable promoter. In a preferred embodiment, the NME protein that is introduced into an animal to increase engraftment of human stem cells or cancer cells is human NME7. In a yet more preferred embodiment, the NME7 protein is a fragment that is ˜33 kDa. In a still more preferred embodiment, the NME protein is human NME7-AB.

Others have reported that inhibitors called ‘2i’ (Silva J et al 2008) and ‘5i’ (Theunissen T W et al 2014) are able to maintain stem cells in naïve-like state. Treatment with the ‘2i’ inhibitors or ‘5i’ inhibitors caused stem cells to revert to a more naïve state. 2i refers to inhibitors of the MAP kinase pathway and GSK3 inhibitors such as PD0325901 and CHIR99021. However, these and other methods that depend on the use of biochemical inhibitors have not satisfied the criteria for being naïve, such as being able to integrate into inner cell mass of other species and in addition they report that they cannot propagate the stem cells for 10 or more passages without either rampant spontaneous differentiation or abnormal karyotype or both.

Naïve State.

Other agents have been reported to maintain stem cells in the naïve state or revert primed stem cells to the naïve state. Chromatin re-arrangement factors MBD3 and CHD4 were recently reported to block the induction of pluripotency (Rais Y et al, 2013). For example, siRNA suppression of the chromatin re-arrangement factors MBD3 and CHD4 were shown to be key components of a method for reverting human primed stem cells to the naïve state. Transcription factors BRD4 and co-factor JMJD6 reportedly suppress NME7 and up-regulate NME1 (Lui W et al, 2013). We found that these factors were expressed at lower levels in naïve stem cells than they were in the later stage primed stem cells. We observed that these four (4) genes, MBD3, CHD4, BRD4 and JMJD6, are naturally suppressed in cancer cells that were cultured in NME1 dimers, NME7 or NME7-AB or NME7-X1 (FIG. 3).

We have demonstrated that human NME1 dimers, also called NM23-H1, bacterial NME1, NME6, NME7-X1 and NME7-AB promote the growth of embryonic stem cells and induced pluripotent stem cells, inhibit their differentiation and maintain them in a naïve state as evidenced by global genetic analysis, having both X chromosomes in the active state if stem cell donor is human and by having the ability to form teratomas in a host animal.

Several examples have been presented here that indicate that contacting cells with an agent or agents that are able to revert stem cells from the primed state to the less mature naïve state are also able to revert a wide variety of cell types to a less mature state: somatic cells to stem or progenitor cells and stem cells back to naïve state.

In a preferred embodiment, a transgenic animal that expresses human NME7 or NME7-AB is generated. Because NME1, human or bacterial, and NME7 inhibit differentiation of stem cells, it may be advantageous to use technology in which the timing of expression of the NME protein, preferably NME7 or NME7-antibody, in the transgenic animal can be controlled. It would be advantageous to have the human NME7 on an inducible promoter, for example to avoid potential problems of NME7 expression during development of the animal. Methods for making the expression of foreign genes inducible in the host animal are known to those skilled in the art. Expression of NME7 or NME7-AB can be inducible using any one of many methods for controlling expression of transgenes that are known in the art.

Alternatively, the expression or timing of expression, of NME7 may be controlled by the expression of another gene which may be naturally expressed by the mammal. For example, it may be desirable for the NME7 or NME7 variant to be expressed in a certain tissue, such as the heart. The gene for NME7 is then operably linked to the expression of a protein expressed in the heart such as MHC. In this instance, the expression of NME7 is turned off when and where the MHC gene product is expressed. Similarly, one may want to have the expression of human NME1, NME6 or NME7 turn on or off in the prostate such that the location and timing of its expression is controlled by the expression of for example, a prostate specific protein. Similarly, the expression of human NME6 or NME7 in a non-human mammal can be controlled by genes expressed in mammary tissues. For example, in a transgenic mouse, human NME6 or human NME7 is expressed from the prolactin promoter, or a similar gene. In this way, it would be possible to induce or repress expression of the human NME protein in a site specific manner

Animals xenografted with human tumors and also injected with human NME7 developed metastatic cancers. Therefore, an animal model for the development of cancer metastasis is generated by making a transgenic animal that expresses human NME7 or more preferably NME7-AB. Optimally the NME7 is on an inducible promoter to allow the animal to correctly develop. Alternatively, a metastatic animal model, preferably rodent, is made by making a transgenic animal that expresses human NME or human NME7 or NME7-AB. Alternatively, the animal is a transgenic animal in which the kinases inhibited by 2i or 5i are suppressed via inducible promoters or agents to suppress the kinases are administered to the test animal. Metastatic animal models are then used to study the basic science of the development or progression of cancers as well as to test the effects of compounds, biologicals, drugs and the like on the development of cancers.

Generation of Animals that Express Human Tissue

Other applications are envisioned wherein an animal transgenic for human NME1, bacterial NME1 or human NME7, preferable NME7-AB, is implanted or engrafted with human cells which may be stem cells or progenitor cells or incipient mesodermal cells. For example in some cases it is desirable to generate an animal, such as a mouse, pig, sheep, bovine animals and primates, that will grow human tissue in its heart, liver, skin or other organ.

One method for doing so is to generate a kind of chimeric animal by implanting human stem cells into an animal that has been made to express human NME7 or human NME7-AB. The human stem cells or progenitor cells can be implanted at various stages of the animal's development, including in vitro and in vivo, at the blastocyst, embryo or fetus stage of development. Because NME7 inhibits differentiation, the NME7 or NME7-AB transgene would be linked to a method by which the timing of its expression is controllable. Methods are known to those skilled in the art which could be used such that expression of the human NME7 or NME7-AB is turned off or decreased at times or locations where it is desirable to have differentiation or maturation occur. One method for making the transgene, preferably NME7, inducible or repressible is to link its expression or repression to the expression of a gene that is only expressed later in development. In such cases, one would make a transgenic animal in which expression of NME7 or NME7-AB is linked to the expression of a later gene expressed in heart or in heart progenitor cells. Thus, the expression or timing of expression, of NME7 is controlled by the expression of another gene which may be naturally expressed by the mammal. For example, it may be desirable for the NME7 or NME7 variant to be expressed in a certain tissue, such as the heart. The gene for the NME7 is then operably linked to the expression of a protein expressed in the heart such as MHC. In this instance, the expression of NME7 is decreased or turned off when and where the MHC gene product is expressed. Similarly, one may want to have the expression of human NME1, NME6 or NME7 turn on in the prostate such that the location and timing of its expression is controlled by the expression of for example, a prostate specific protein. Similarly, the expression of human NME1 or NME7 in a non-human mammal can be controlled by genes expressed in mammary tissues. For example, in a transgenic mouse, human NME1 or human NME7 can be expressed or repressed by the prolactin promoter, or a similar gene.

In this way, an animal transgenic for human NME7 or NME7-AB can be allowed to grow to a point, then implanted with human stem or progenitor cells, where they proliferate because of contact with human NME protein. The expression of the human NME is then turned off such that a specific organ or part of an organ in the animal would develop as a human tissue.

As an aside, it is also contemplated that primates or any animal which shares close global sequence identity to humans may not be a good host animal candidate as cross-species interaction may occur and thus ethical issues may arise.

The invention contemplates many applications of animals transgenic for human NME1, bacterial NME1 or human NME7, or NME7-AB. In one aspect of the invention, human stem or progenitor cells are implanted in the NME transgenic animal or germ cells of what will be a transgenic animal Expression of the NME may be inducible or repressible. Depending on the site and timing of the implantation of the stem or progenitor cells, the resulted animal can be made to express human heart, liver, neuronal cells or skin.

Thus human tissues can be generated in a transgenic non-human mammal, wherein the mammal expresses human MUC1 or MUC1* or NME protein in the germ cells or somatic cells, wherein the germ cells or somatic cells contain a recombinant human MUC1 or MUC1* or NME gene sequence introduced into said mammal, wherein the expression of the gene sequence can be induced or repressed either by introduction of an external composition or by linking its expression or repression to the expression or repression of a naturally occurring gene of the host animal Stem cells or progenitor cells that are xenogeneic in origin to the non-human mammal are transferred to the transgenic animal such that the gene is induced to be expressed so as to multiply the stem or progenitor cells and then repressing the gene expression so as to generate tissue from the xenografted stem cells. One method by which repression of the transgene is carried out is by contacting the stem cell or progenitor cells with a tissue differentiation factor. Transgene repression is also carried out naturally in the mammal in response to naturally produced host tissue differentiation factors.

These animals can be used for drug discovery. They can also be used for toxicity testing, to use an animal to determine the effects of a compound, biological or drug on human tissue or on the development of human tissue. Alternatively, the transgenic animal implanted with human stem or progenitor cells is used to grow human tissue for transplant into a human patient. In some cases, the stem or progenitor cells that are implanted are from a patient who will be the recipient of the human tissue harvested from the transgenic animal.

In one aspect, the MUC1, MUC1* or NME protein expression may be induced until the amount of transferred stem or progenitor cells are sufficiently large. The MUC1, MUC1* or NME protein expression may then be shut down by injecting the host mammal with a substance that represses the expression of MUC1, MUC1* or NME protein. The population of stem or progenitor cells may be induced to differentiate by either natural methods such as by the expression in the mouse of a differentiation inducing factor for a particular tissue or organ type, or chemical or protein substances may be injected into the host at the site of stem or progenitor cell transference to cause differentiation to desired tissue type.

Induction, differentiation/transformation agents for endoderm cell tissue may include without limitation the following agents: hepatocyte growth factor, oncostatin-M, epidermal growth factor, fibroblast growth factor-4, basic-fibroblast growth factor, insulin, transferrin, selenius acid, BSA, linoleic acid, ascorbate 2-phosphate, VEGF, and dexamethasone, for the following cell types: liver, lung, pancreas, thyroid, and intestine cells.

Induction, differentiation/transformation agents for mesoderm tissue include without limitation the following agents: insulin, transferrin, selenous acid, BSA, linoleic acid, TGF-β1, TGF-β3, ascorbate 2-phosphate, dexamethasone, β-glycerophosphate, ascorbate 2-phosphate, BMP, and indomethacin, for the following cell types: cartilage, bone, adipose, muscle, and blood cells.

Induction, differentiation/transformation agents for ectoderm tissue include without limitation the following agents: dibutyryl cyclin AMP, isobutyl methylxanthine, human epidermal growth factor, basic fibroblast growth factor, fibroblast growth factor-8, brain-derived neurotrophic factor, and/or other neurotrophic growth factor, for the following cell types: neural, skin, brain, and eye cells.

Regulators of NME Protein or Downstream Effectors of NME Protein can Substitute for the NME Protein

These studies have shown that one way in which NME proteins function to promote stem-like or cancer-like growth is by binding to a clipped form of the MUC1 transmembrane protein, herein referred to as MUC1*, which consists primarily of the PSMGFR sequence. Dimerization of the MUC1* extracellular domain stimulates growth and de-differentiation of somatic cells, stem cells and cancer cells, making them more metastatic.

Another way that NME proteins exert their effects is by being transported to the nucleus where they function directly or indirectly to stimulate or suppress other genes. It has been previously reported (Boyer et al, 2005) that OCT4 and SOX2 bind to the promoter sites of MUC1 and its cleavage enzyme MMP16. The same study reported that SOX2 and NANOG bind to the promoter site of NME7. We conclude, on the basis of our experiments that these ‘Yamanaka’ pluripotency factors (Takahashi and Yamanaka, 2006) up-regulate MUC1, its cleavage enzyme MMP16 and its activating ligand NME7. It has also been previously reported that BRD4 suppresses NME7, while its co-factor JMJD6 up-regulates NME1 (Thompson et al), which we have demonstrated is a self-regulating stem cell growth factor that is expressed later than NME7 in embryogenesis. Still others recently reported that siRNA suppression of Mbd3 or Chd4 greatly reduced resistance to iPS generation (Rais Y et al 2013 et al.) and was able to maintain stem cells in the naïve state. Evidence presented here shows that there is a reciprocal feedback loop wherein NME7 suppresses BRD4 and JMJD6, while also suppressing inhibitors of pluripotency Mbd3 and CHD4. We note that in naïve human stem cells, these four factors BRD4, JMJD6, Mbd3 and CHD4 are suppressed compared to their expression in later stage ‘primed’ stem cells. We also note that the 2i inhibitors (inhibitors of Gsk3β and MEK) that revert mouse primed stem cells to the naïve state, also down regulated the same four factors BRD4, JMJD6, Mbd3 and CHD4.

We have also discovered that NME7 up-regulates SOX2 (>150×), NANOG (˜10×), OCT4 (˜50×), KLF4 (4×) and MUC1 (10×). Importantly, we have shown that NME7 up-regulates cancer stem cell markers including CXCR4 (˜200×) and E-cadherin (CDH1). Taken together these multiple lines of evidence point to the conclusion that NME7 is the most primitive stem cell growth and pluripotency mediator and that it is a powerful factor in the transformation of somatic cells to a cancerous state as well as transforming cancer cells to the more metastatic cancer stem cells.

Therefore, the present invention contemplates substituting genes and gene products that increase expression of NME7 for NME7. Similarly, the invention contemplates substituting downstream effectors of NME7 for NME7. For example, alone or in combination, agents that suppress MBD3, CHD4, BRD4 or JMJD6 can be substituted in any of the methods described herein, for NME7, which we have shown suppresses MBD3, CHD4, BRD4 or JMJD6.

Stem Cell-Based Organ and Tissue Generation

The present invention discloses methods for generating, maintaining or proliferating human stem cells in a naïve state and using the resultant cells in a non-human host animal or a fertilized egg, blastocyst or embryo of a non-human animal in order to generate chimeric organisms or animals that are comprised of DNA of the non-human host and DNA of the human donor stem cells. Limbs, nerves, blood vessels, tissues, organs, or factors made in them, or secreted from them, of a chimeric species that contains some human DNA, can be harvested for several uses including transplant into humans, administration into humans for medicinal benefit, including anti-aging, and scientific experiments including drug testing and disease modeling.

In a first method, human naïve state stem cells are generated, maintained or proliferated by contacting human primed state stem cells with an NME family protein or an agent that dimerizes the MUC1* growth factor receptor.

In a second method, human naïve state stem cells are generated, maintained or proliferated by inducing somatic cells to revert to a less mature state, such as through the use of iPS technologies, wherein cells are reprogrammed in the presence of an NME family protein or an agent that dimerizes the MUC1* growth factor receptor.

In a third method, human naïve state stem cells are generated, maintained or proliferated by culturing cells obtained from a human embryo, blastocyst or fertilized egg in the presence of an NME family protein or an agent that dimerizes the MUC1* growth factor receptor.

Said NME proteins or agents that dimerize MUC1* convert human primed state stem cells to a naïve state. Said NME proteins or agents that dimerize MUC1* also support the derivation of naïve state embryonic stem cell lines from cells taken from a human embryo. Said NME proteins or agents that dimerize MUC1* support the generation of naïve state induced pluripotent stem cells lines, wherein differentiated cells are reprogrammed to a stem cell state.

In a preferred embodiment, the NME family protein is NME7. In a still more preferred embodiment the NME family member is NME7-AB or NME7-X1 or other isoform or truncation of NME7. In another embodiment the NME family member is dimeric NM23, aka NME1. In yet another embodiment, the NME family member is NME6. In a preferred embodiment the agent that dimerizes MUC1* is an antibody that binds to the PSMGFR peptide of the MUC1* extracellular domain.

In one aspect of the invention, naïve state human stem cells, generated by a method that includes contacting human cells with NME1 dimers, NME6 dimers, NME7, NME7-AB or NME7-X1, then inserting or injecting said cells into a morula, blastocyst, embryo or fetus of a non-human animal. Chimeric animals are generated that have some tissues, organs or other body parts that are at least in part of human origin and emanated from the human stem cells inserted into the blastocyst or embryo. The tissues, organs or other body parts are harvested from the host animal when completely developed or at any earlier stage of development. The tissues, organs, or body parts or factors generated by these human body parts are then transplanted into or administered to a human recipient in need of a new organ or in need of regenerative properties of factors secreted by the human tissues or organs of the host non-human.

In one aspect of the invention, the cells of the non-human animal have been genetically altered or treated for example with biochemical inhibitors such that the developing non-human animal is not able to generate certain tissues or organs. In this aspect, the chimeric animal would generate certain tissues or organs that emanate from, or have significant contribution from, the human donor stem cells and will be partially or entirely human.

In one aspect of the invention, the donor stem cells are from a donor in need of the tissue or organ that is generated in the non-human animal and at some stage of development or after the animal is mature, the tissue or organ is harvested and transplanted into the donor human. In another aspect of the invention, the donor stem cells are from a donor who is not the intended recipient of the tissues, organs or other material generated in the chimeric species. In one aspect the donor stem cells are iPS cells and in another aspect the stem cells are embryonic stem cells.

In one aspect of the invention, stem cells are cultured in a media containing an NME protein. The NME protein can be dimeric NME1, dimeric NME6, NME7, dimeric B domain of NME7, NME7-X1 or NME7-AB. In a preferred embodiment the NME protein is dimeric NME1.

In a more preferred embodiment the NME protein is NME7-X1. In a still more preferred embodiment, the NME protein is NME7-AB. In some cases, stem cell attachment to a surface is facilitated by coating said surface with an anti-MUC1* antibody wherein the antibody has the ability to bind to a peptide comprising at least 15 contiguous amino acids of the PSMGFR sequence. In another case, stem cell attachment to a surface is facilitated by coating said surface with an NME protein, which in some cases may be histidine-tagged and coated onto a surface presenting a metal-chelate-metal moiety such as nitrile-tri-acetic acid-Nickel, aka NTA-Ni++. In other instances, stem cell attachment to a surface is facilitated by coating said surface with an integrin or integrin fragment wherein the integrin is vitronectin, fibrinectin, collagen and the like. In other instances, stem cell attachment to a surface is facilitated by coating said surface with peptides, small molecules or polymers. In some cases a Rho I kinase inhibitor is added to the culture media to further enhance surface attachment.

The generation, induction or maintenance of stem cells is achieved according to parts or all of the methods described above. However, for the generation, induction or maintenance of non-human stem cells, it may be advantageous to contact the cells with an NME protein whose sequence is that of the non-human species. For example, for generating, inducing pluripotency or maintaining pig stem cells, it may be advantageous to use an NME protein whose sequence is that of native pig NME6, NME1, NME7, NME7-X1 or NME7-AB. Facilitate surface attachment, it may be advantageous to coat the surface with an antibody selected for its ability to bind to a peptide comprising at least 15 contiguous amino acids of the PSMGFR region of the MUC1* extracellular domain, wherein the sequence of the peptide is the native sequence of pig MUC1* extracellular domain.

### EXAMPLES

### Example 1

Minimal Media

Serum-free Minimal Media 500 mL includes the following components:

### 394 mL DMEM/F12, GlutaMAX; 100 mL Knockout™ Serum Replacement; 5.0 mL 100× MEM Non-essential Amino Acid Solution;

0.9 mL β-mercaptoethanol, 55 mM stock.

When a rho kinase inhibitor, “Ri” or “ROCi”, was added it was Y27632 from Stemgent (Cambridge, Mass.), added immediately before use to a final concentration of 10 uM.

### Example 2

Culturing Stem Cells in NME Media

To serum-free Minimal Media, described in Example 1, add one of the following NME proteins: 8 nM (final concentration) dimeric rhNME1 (aka NM23) preferably having the S120G mutation to ensure stable dimers, 8 nM dimeric NME6, 8 nM bacterial HSP593 recombinant NME1, 4 nM NME7_{AB} or 4 nM NME7-X1. Stem cells can be grown in suspension in an NME media or on cell culture plates. If stem cells are being cultured on cell culture plates coated with an anti-MUC1* antibody such as MN-C3, then a Rho Kinase inhibitor was added such as Y-26732 to a final concentration of 10 uM.

Cell culture plates were prepared at least 1 day before stem cell plating. Cell culture plates were coated with a solution containing ˜12.5 ug/mL of MN-C3 anti-MUC1* antibody. The coated plates were incubated overnight at 4 degrees before stem cells were plated onto to them, and without a pre-wash step. Stem cells were plated at densities broadly corresponding to the density obtained when 100,000 cells to 300,000 cells are plated per well of a 6-well plate. Cells are suspended in NME Media to which was added a Rho kinase inhibitor. Cells were incubated undisturbed in a 5% CO2/5%02 incubator for 48 hours. Thereafter, media was changed every 24 or 48 hours until cells reached ˜80% confluency (FIG. 20B). Cells were dissociated to single cells using Trypsin/EDTA or Tryp1E. Repeat process from beginning to expand.

### Example 3

iPS Generation in NME Media

Day −2 (48 hours before reprogramming): Fibroblasts were seeded onto standard tissue culture-treated 6-well plates in 2 mLs per well of Fibroblast Medium (DMEM High Glucose with glutamine, 10% FBS), at 25,000-100,000 cells per well. Culture for 48 hours in 5% CO₂.
Day 0: Fibroblast Media was changed to NME Media (Example Fibroblasts were transduced with master reprogramming factors, such as Yamanaka factors or Thomson factors, according to standard protocol. Any method of introducing nucleic acids that will cause expression of OCT4, SOX2, NANOG or KLF4 and c-Myc if desired will suffice. Common methods use non-integrating viral delivery systems such as lend virus, Sendai virus, gamma retrovirus or transposons such as Sleeping Beauty.
Day 1: Cells were washed with Minimal Media to remove virus and cell debris, then replaced with 2 mL-4 mL per well of NME Media, without Rho kinase inhibitor.
Day 3: Change media with 2 mL to 4 mL per well of NME Media, without Rho kinase inhibitor.
Day 5: Change media with 2 mL to 4 mL per well of NME Media, without Rho kinase inhibitor.
Day 6: Cell culture plates were prepared with an anti-MUC1* antibody such as MN-C3 coated onto cell culture plates at a concentration of 3.25 ug/mL to 24 ug/mL with about 12.5 ug/mL preferred and incubate the antibody coated plates at 4° C. overnight.
Day 7: Transduced cells, which were changing morphology to that of stem cells by this time, were dissociated with Trypsin/EDTA, passed through a cell strainer, and seeded onto MN-C3 coated plates in NME Media plus a Rho kinase inhibitor (such as 10 uM Y-26732). The reprogrammed cells were then plated at 5×10⁴-1×10⁵ per well. From this point onward, cells are cultured in an NME media with a Rho kinase inhibitor such as 10 uM Y-26732 added and incubated for first 48 hours undisturbed in 5% CO₂/5% O₂.
Day 9 and onward: Change media daily using the same NME media throughout plus Rho kinase inhibitor such as 10 uM Y-26732.
Day 16-Day 21: Colonies were picked and each clone was cultured on MN-C3 coated surfaces, first in 96-well plates, then 24-well, then 12-well, then 6-well and larger formats after stem cells were characterized and found to express all normal pluripotency genes, naïve genes, formed teratomas when implanted into animals and had normal karyotype.
iPS cells were also generated from blood using same process as Day 1 and onward.

In the cells shown in FIGS. 21A, 21B and 21C, neonatal male fibroblasts were used. In FIG. 21C the NME Media used was Minimal Media with 4 nM NME7-AB.

### Example 4

Reprogramming Capability of NME Proteins in the Absence of Master Pluripotency Regulators OCT4, SOX2, KLF4 or c-Myc

In this example, fibroblast cells were cultured in Minimal Media to which was added recombinant human NME1/NM23 dimers, bacterial HSP593 NME1 dimers or human recombinant NME7-AB. As a control, the fibroblasts were cultured in their normal media, which is for 500 mL, 445 mL DMEM high glucose base media, 5 mL GlutaMAX and 50 mL of fetal bovine serum (FBS). After 15-20 days in culture in Minimal Media with either NME1/NM23 dimers, bacterial HSP593 NME1 dimers or NME7-AB, RT-PCR showed that the resultant cells greatly increased expression of stem cell marker genes OCT4 and NANOG, see FIG. 1. Just as the cancer cells had, they also decreased expression of BRD4, JMJD6, MBD3 and CHD4. FIG. 2 shows a graph of RT-PCR measurements of the expression of genes that code for the chromatin rearrangement factors BRD4, JMJD6, MBD3 and CHD4. FIG. 3 shows a graph of RT-PCR measurements of the expression of pluripotency genes, genes that code for chromatin rearrangement factors BRD4, JMJD6, MBD3 and CHD4 and NME proteins. Here, ‘minus ROCi’ refers to cells that became non-adherent and floated off the surface. The morphology of the cells also completely changed so that they no longer were recognizable as fibroblasts and look like stem cells (FIGS. 8-11).

### Example 5

NME7-AB cultured human stem cells incorporate into inner cell mass of mouse morula. Mouse eggs were fertilized in vitro. At Day 2.5 post fertilization, 10 human stem cells that had been generated in and cultured in NME7-AB were separately injected into the fertilized eggs. Day 2.5 is before the inner cell mass has formed. 48 hours later, at Day 4.5, morulas were stained with a fluorescent antibody that stains human Tra 1-81. in some of the figures, arrows point to human naïve NME7-AB cells that have incorporated into the inner cell mass, which indicates the development of a chimeric animal See FIGS. 22A-22D, FIGS. 23A-23J and FIGS. 24A-24J which show confocal images of other mouse blastocysts into which human NME7-naïve cells were injected at Day 2.5 and also show integration into inner cell mass of blastocyst at Day 4.5. See also FIGS. 27A-27F, FIGS. 28A-26J, FIGS. 29A-29J, and FIGS. 30A-30J which also show human NME7-AB grown naïve stem cells injected at Day 2.5 into a mouse morula, are found concentrated in the inner cell mass of the blastocyst 48 hours later at Day 4.5. As a control, FGF-grown primed state human stem cells were injected into fertilized eggs at Day 2.5 and at Day 4.5 stained with anti-human Tra 1-81 and also with CDX2 which stains non-inner cell mass region called the Trophoectoderm. FIGS. 25A-25D and 26A-26H show that these primed state cells did not incorporate into the inner cell mass but are in the trophectoderm.

### Example 6

NME7-AB cultured human stem cells were transfected with a red fluorophore called tomato red or TDtomato. These fluorescent human naïve cells were then injected into Day 2.5 fertilized mouse eggs and imaged at Day 4.5. The morulas were also stained with DAPI and a fluorescent antibody that stains the trophectederm. See FIGS. 27A-27F, FIGS. 28A-26J, FIGS. 29A-29J, and FIGS. 30A-30J show human NME7-AB grown naïve stem cells injected at Day 2.5 into a mouse morula, are found concentrated in the inner cell mass of the blastocyst 48 hours later at Day 4.5. Arrows indicate where the human cells incorporated into the inner cell mass, indicating formation of a chimeric animal.

### Example 7

Non-human primate species stem cells were generated in NME7-AB media in the absence of bFGF. Rhesus macaque and crab-eating macaque fibroblasts were transfected with core pluripotency factors in a media containing NME7-AB and in the absence of bFGF or feeder cells. In this case the Yamanaka factors Oct 4, Sox2, Klf4, and c-Myc were used but could be substituted for other pluripotency factors, either genes or gene products. Between Day 5 and 7 post gene transfection, when cells began to detach from the surface, cells were re-plated onto a surface coated with an anti-MUC1* antibody, in this case MN-C3. Beginning on Day 6 for crab-eating macaques and Day 14 for rhesus macaques, colonies began to appear. FIGS. 31A-31F show photograph of control plates containing fibroblasts from crab-eating macaques in culture in NME7-AB for 6 days but without transduction of Yamanaka pluripotency factors OCT4, SOX2, NANOG and c-Myc. FIGS. 32A-32C, FIGS. 33A-33F, and FIGS. 34A-34F show fibroblasts from crab-eating macaques being reprogrammed by Yamanaka factors and NME7-AB. After Day6, emerging iPS colonies are transferred to an anti-MUC1* antibody surface, where they continue to expand until about Day 14-17 when individual clones are picked and expanded. To our knowledge, this is the first time scientists succeeded in generating non-human primate iPS cells in the absence of mouse or human feeder cells. Researchers experience extreme difficulty culturing non-human primate stem cells. They differentiate spontaneously and do not grow well in FGF-based media. We overcame all these problems when we moved non-human primate cells into NME7-AB media, shown in FIGS. 35A-35D, FIGS. 36A-36D, FIGS. 37A-37B, and FIGS. 38A-38H. Generation of rhesus macaque iPS cells by transduction of Yamanaka factors or Thomson factors in serum-free, FGF-free, NME7-AB media over MN-C3 antibody surface or over MEFs is shown at various stages in FIGS. 39A-39C, FIGS. 40A-40C, FIGS. 41A-41D, and FIGS. 42A-42B. Whether fibroblasts were reprogrammed on MN-C2 antibody surfaces or on MEFs was not critical but more colonies were generated when the surface was the anti-MUC1* antibody surface.

### Example 8

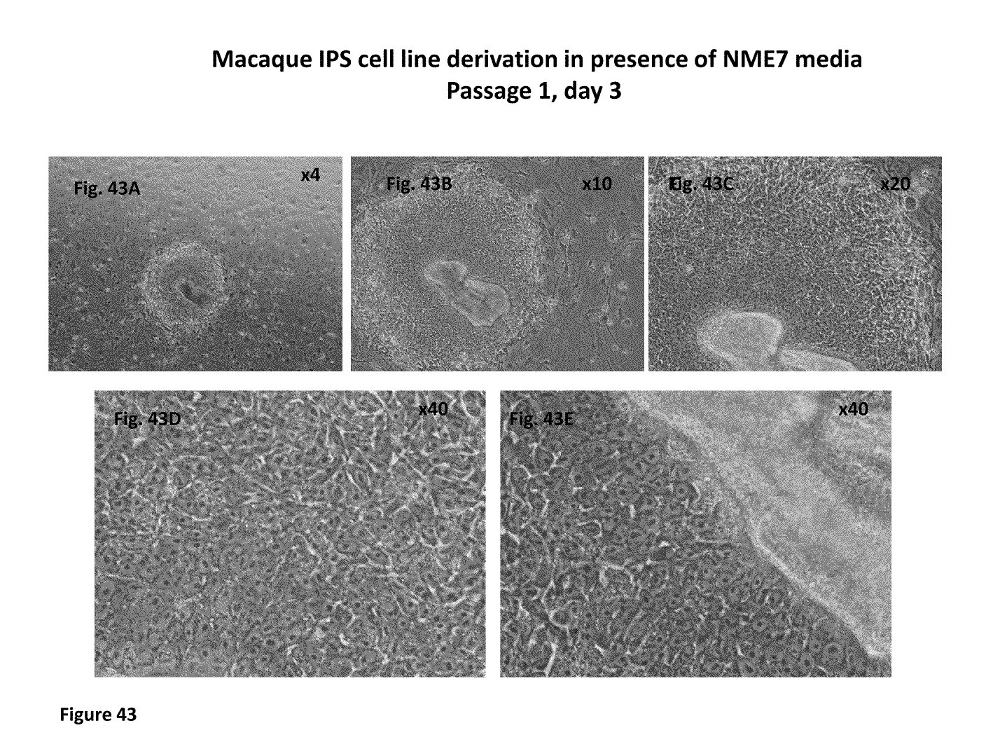

Primate species embryonic stem cells proliferate and are maintained in NME7-AB media. After colonies were picked, they were replated onto MN-C3 antibody coated surfaces or onto MEFs and could be serially passaged indefinitely in a serum-free media containing NME7-AB at a concentration between 2 nM and 32 nM wherein 4 nM worked best. See FIGS. 43A-43E, FIGS. 44A-44F, FIGS. 45A-45H, FIGS. 46A-46H, FIGS. 47A-47G, and FIGS. 48A-48D.

All of the references cited herein are incorporated by reference in their entirety.

### CITED REFERENCES LIST

Clarke M F, Dick J E, Dirks P B, Eaves C J, Jamieson C H, Jones D L, Visvader J, Weissman I L, Wahl G M. (2006) Cancer stem cells—perspectives on current status and future directions: AACR Workshop on cancer stem cell. Cancer Res. October 1; 66(19):9339-44. Epub 2006 Sep. 21.
Chen K, Huang Y H, Chen J L. (2013) Understanding and targeting cancer stem cells:
therapeutic implications and challenges. Acta Pharmacologica Sinica 34: 732-740; Review
Darash-Yahana M, Pikarsky E, Abramovitch R, Zeira E, Pal B, Karplus R, Beider K, Avniel S, Kasem S, Galun E, Peled A (2004) Role of high expression levels of CXCR4 in tumor growth, vascularization, and metastasis. FASEB J 18(11): 1240-1242
Mahanta S, Fessler S, Park J, Bamdad C. A Minimal Fragment of MUC1 Mediates Growth of Cancer Cells, 2008 PLoS ONE 3:e2054-2065.
Hikita S, Clegg O, Kosik K, Bamdad C. MUC1* Mediates the Growth of Human Pluripotent Stem Cells, 2008 PLoS ONE 3:e3312-3325.
Kumar S M, Liu S, Lu H, Zhang H, Zhang P J, Gimotty P A, Guerra M, Guo W, Xu X. (2012) Acquired cancer stem cell phenotypes through Oct4-mediated dedifferentiation. Oncogene. November 22; 31(47):4898-911.
Liu K, Lin B, Zhao M, Yang X, Chen M, Gao A, Liu F, Que J, Lan X. (2013) The multiple roles for Sox2 in stem cell maintenance and tumorigenesis. Cellular Signaling May; 25(5):1264-71. Review
Yeo J C, Jiang J, Tan Z Y, Yim G R, Ng J H, Göke J, Kraus P, Liang H, Gonzales K A, Chong H C, Tan C P, Lim Y S, Tan N S, Lufkin T, Ng H H. (2014) Klf2 is an essential factor that sustains ground state pluripotency. Cell Stem Cell. June 5; 14(6):864-72.
Oshima N, Yamada Y, Nagayama S, Kawada K, Hasegawa S, Okabe H, Sakai Y, Aoi T. (2014) Induction of cancer stem cell properties in colon cancer cells by defined factors. PLoS One. July 9; 9(7):e101735
Wang M L, Chiou S H, Wu C W. (2013) Targeting cancer stem cells: emerging role of Nanog transcription factor. Onco targets and Therapy. September 4; 6:1207-20. Review.
Xu C, Rosier E, Jiang J, Lebkowski J S, Gold J D, et al. (2005) Basic Fibroblast Growth Factor Supports Undifferentiated Human Embryonic Stem Cell Growth Without Conditioned Medium. STEM CELLS 23: 315-323.
Fessler S, Wotkowicz M, Mahanta S, Bamdad C (2009) MUC1* is a determinant of trastuzumab (Herceptin) resistance in breast cancer cells, Breast Cancer Res Treat 118:113-124 DOI 10.1007/s10549-009-0412-3
Lissa Nurrul Abdullah and Edward Kai-Hua Chow (2013) Mechanisms of chemoresistance in cancer stem cells. Clinical and Translational Medicine January 17; 2(1):3
Miki J, Furusato B, Li H, Gu Y, Takahashi H, Egawa S, Sesterhenn I A, McLeod D G, Srivastava S, Rhim J S. Identification of putative stem cell markers, CD133 and CXCR4, in hTERT-immortalized primary nonmalignant and malignant tumor-derived human prostate epithelial cell lines and in prostate cancer specimens. Cancer Res. 2007 Apr. 1; 67(7):3153-61.
Jeter C R, Liu B, Liu X, Chen X, Liu C, Calhoun-Davis T, Repass J, Zaehres H, Shen J J, Tang D G. NANOG promotes cancer stem cell characteristics and prostate cancer resistance to androgen deprivation. Oncogene. 2011 Sep. 8; 30(36):3833-45. PMCID:
Hong X, Chedid K, Kalkanis S N. Glioblastoma cell line-derived spheres in serum-containing medium versus serum-free medium: a comparison of cancer stem cell properties. Int. J. Oncol. 2012 November; 41(5):1693-700.
Faber A, Goessler U R, Hoermann K, Schultz J D, Umbreit C, Stern-Straeter J. SDF-1-CXCR4 axis: cell trafficking in the cancer stem cell niche of head and neck squamous cell carcinoma. Oncol. Rep. 2013 June; 29(6):2325-31.
Mukherjee D, Zhao J. The Role of chemokine receptor CXCR4 in breast cancer metastasis. Am J Cancer Res. 2013; 3(1):46-57. PMCID: PMC3555200
Herreros-Villanueva M, Zhang J-S, Koenig A, Abel E V, Smyrk T C, Bamlet W R, de Narvaj as A A-M, Gomez T S, Simeone D M, Bujanda L, Billadeau D D. SOX2 promotes dedifferentiation and imparts stem cell-like features to pancreatic cancer cells. Oncogenesis. 2013; 2:e61. PMCID: PMC3759123
Sefah K, Bae K-M, Phillips J A, Siemann D W, Su Z, McClellan S, Vieweg J, Tan W. Cell-based selection provides novel molecular probes for cancer stem cells. Int. J. Cancer. 2013 Jun. 1; 132(11):2578-88.
Su H-T, Weng C-C, Hsiao P-J, Chen L-H, Kuo T-L, Chen Y-W, Kuo K-K, Cheng K-H. Stem cell marker nestin is critical for TGF-β1-mediated tumor progression in pancreatic cancer. Mol. Cancer Res. 2013 July; 11(7):768-79.
Nichols J, Smith A (2009) Naive and primed pluripotent states. Cell Stem Cell 4: 487-492.
Hanna J, Cheng A W, Saha K, Kim J, Lengner C J, et al. (2010) Human embryonic stem cells with biological and epigenetic characteristics similar to those of mouse ESCs. Proc Natl Acad Sci USA 107: 9222-9227.
Smagghe, B. J. Stewart A. K., Carter M. G., Shelton L. S., Bernier K. J., Hartman E. J., Calhoun A. K., Hatziioannou V. M., Lillacci G., Kirk B. A., DiNardo B. A., Kosik K. S., Bamdad C. (2013) MUC1* Ligand, NM23-H1, Is a Novel Growth Factor That Maintains Human Stem Cells in a More Naïve State. PLoS ONE 8(3): e58601
Silva J, Barrandon O, Nichols J, Kawaguchi J, Theunissen T W, Smith A (2008). Promotion of reprogramming to ground state pluripotency by signal inhibition. PLoS Biol. 21; 6(10)
Theunissen T W, Powell B E, Wang H, Mitalipova M, Faddah D A, Reddy J, Fan Z P, Maetzel D, Ganz K, Shi L, Lungjangwa T, Imsoonthornruksa S, Stelzer Y, Rangarajan S, D'Alessio A, Zhang J, Gao Q, Dawlaty M M, Young R A, Gray N S, Jaenisch R. (2014) Systematic Identification of Culture Conditions for Induction and Maintenance of Naive Human Pluripotency. Cell Stem Cell. 2014 Jul. 24, S1934-5909(14)00298-7.
Hugo H J, Kokkinos M I, Blick T, et al. Defining the E-cadherin repressor interactome in epithelial-mesenchymal transition: the PMC42 model as a case study. (2011) Cells Tissues Organs; 193:23-40
Epstein R J (2004) The CXCL12-CXCR4 chemotactic pathway as a target of adjuvant breast cancer therapies. Nat Rev Cancer 4(11): 901-909
Müller, A.; Homey, B.; Soto, H.; Ge, N.; Catron, D.; Buchanan, M. E.; McClanahan, T.; Murphy, E.; Yuan, W.; Wagner, S. N.; Barrera, J. L.; Mohar, A.; Verástegui, E.; Zlotnik, A. (2001) Involvement of chemokine receptors in breast cancer metastasis. Nature, 410 (6824), 50-56.
Rais Y1, Zviran A, Geula S, Gafni O, Chomsky E, Viukov S, Mansour A A, Caspi I, Krupalnik V, Zerbib M, Maza I, Mor N, Baran D, Weinberger L, Jaitin D A, Lara-Astiaso D, Blecher-Gonen R, Shipony Z, Mukamel Z, Hagai T, Gilad S, Amann-Zalcenstein D, Tanay A, Amit I, Novershtern N, Hanna J H (2013). Deterministic direct reprogramming of somatic cells to pluripotency, 502(7469):65-70.
Liu W, Ma Q, Wong K, Li W, Ohgi K, Zhang J, Aggarwal A K, Rosenfeld M G. Brd4 and JMJD6-Associated Anti-Pause Enhancers in Regulation of Transcriptional Pause Release. Cell. 2013 Dec. 19; 155(7):1581-95. PMCID: PMC3886918.
Amit M, Carpenter M K, Inokuma M S, Chiu C-P, Harris C P, et al. (2000) Clonally Derived Human Embryonic Stem Cell Lines Maintain Pluripotency and Proliferative Potential for Prolonged Periods of Culture. Developmental Biology 227: 271-278.
Ludwig T E, Levenstein M E, Jones J M, Berggren W T, Mitchen E R, et al. (2006) Derivation of human embryonic stem cells in defined conditions. Nat Biotechnol 24: 185-187.
Xu R H, Peck R M, Li D S, Feng X, Ludwig T, et al. (2005) Basic FGF and suppression of BMP signaling sustain undifferentiated proliferation of human ES cells. Nat Methods 2: 185-190.
Liu W, Ma Q, Wong K, Li W, Ohgi K, Zhang J, Aggarwal A K, Rosenfeld M G. Brd4 and JMJD6-Associated Anti-Pause Enhancers in Regulation of Transcriptional Pause Release. Cell. 2013 Dec. 19; 155(7):1581-95. PMCID: PMC3886918.
Rais Y, Zviran A, Geula S, Gafni O, Chomsky E, Viukov S, Mansour A A, Caspi I, Krupalnik V, Zerbib M, Maza I, Mor N, Baran D, Weinberger L, Jaitin D A, Lara-Astiaso D, Blecher-Gonen R, Shipony Z, Mukamel Z, Hagai T, Gilad S, Amann-Zalcenstein D, Tanay A, Amit I, Novershtern N, Hanna J H. Deterministic direct reprogramming of somatic cells to pluripotency. 2013 Sep. 18, Nature 502, 65-70 DOI: 10.1038/nature12587
Herreros-Villanueva M, Zhang J-S, Koenig A, Abel E V, Smyrk T C, Bamlet W R, de Narvajas A A-M, Gomez T S, Simeone D M, Bujanda L, Billadeau D D. SOX2 promotes dedifferentiation and imparts stem cell-like features to pancreatic cancer cells. Oncogenesis. 2013; 2:e61. PMCID: PMC3759123
Hong X, Chedid K, Kalkanis S N. Glioblastoma cell line-derived spheres in serum-containing medium versus serum-free medium: a comparison of cancer stem cell properties. Int. J. Oncol. 2012 November; 41(5):1693-700.
Silva J, Barrandon O, Nichols J, Kawaguchi J, Theunissen T W, Smith A. Promotion of reprogramming to ground state pluripotency by signal inhibition. PLoS Biol. 2008 Oct. 21; 6(10):e253. PMCID: PMC2570424
Boyer et al, 2005, “Core Transcriptional Regulatory Circuitry in Human Embryonic Stem Cells”, Cell, Vol. 122, 947-956
Takahashi K and Yamanaka S (2006) Induction of pluripotent stem cells from mouse embryonic and adult fibroblast cultures by defined factors. Cell 126(4):663-676.
Porter D et al. (2011) Chimeric antigen receptor-modified T cells in chronic lymphoid leukemia. N Engl J Med 365:725-733 DOI: 10.1056/NEJMoa1103849
Tiller T et al. (2013) A fully synthetic human Fab antibody library based on fixed VH/VL framework pairings with favorable biophysical properties. MABs 9:5(3) PMID: 23571156
Webb P A, Perisic O, Mendola C E, Backer J M and Williams R L. The crystal structure of a human nucleoside diphosphate kinase, NM23-H2. J Mol Biol. 1995, 251:574-587.
Min K, Song H K, Chang C, Kim S Y, Lee K J and Suh S W. Crystal structure of human nucleoside diphosphate kinase A, a metastasis suppressor. Proteins. 2002, 46:340-342.

Those skilled in the art will recognize, or be able to ascertain using no more than routine experimentation, many equivalents to the specific embodiments of the invention specifically described herein. Such equivalents are intended to be encompassed in the scope of the claims.

1. A method for generating human tissues or organ in a non-human animal host comprising:
(i) generating human naïve state stem cells and injecting them into a fertilized egg, morula, blastocyst, embryo or developing fetus of the non-human animal host such that a chimeric animal is generated;
(ii) harvesting human tissues, organs, cells or factors secreted by or made in the human tissues or cells from the chimeric animal;
(iii) transplanting or administering the harvested material into a human resulting in generation of human tissues.

2. The method as in claim 1, wherein the naïve state stem cells are generated using NME7, NME7-AB, NME7-X1, NME6 or dimeric NME1.

3. The method as in claim 2, wherein the naïve stem cells are iPS cells that have been reprogrammed in a medium containing NME7, NME7-AB, NME6, NME7-X1 or dimeric NME1.

4. The method as in claim 2, wherein the naïve stem cells are embryonic stem cells that have been cultured in a medium containing NME7, NME7-AB, NME6, NME7-X1 or dimeric NME1.

5. The method as in claim 1, wherein the non-human cells of the blastocyst or embryo have been genetically altered.

6. The method as in claim 5, wherein the genetic alteration results in the host animal being unable to generate a certain tissue or organ.

7. The method according to claim 1, wherein the agent that maintains stem cells in the naïve state or reverts primed stem cells to the naïve state is an NME protein, 2i, 5i, chemical, or nucleic acid.

8. The method according to claim 7, wherein the NME protein is NME1 dimer, NME7 monomer, NME7-AB, NME6 dimer, or bacterial NME.

9. The method according to claim 1, wherein non-human animal is a rodent, pig bovine, sheep or primate.

10. The method according to claim 9, wherein the rodent is a mouse or rat.

11. The method according to claim 3, wherein the NME protein is present in serum free media as the single growth factor.

12. The method of claim 1, wherein the non-human animal host expresses NME protein having a sequence that is homologous to the native sequence of the species of the stem cells to be generated.

13. The method according to claim 12, wherein the NME protein is NME7, NME7-AB, NME7-X1, or dimeric NME1 or NME6.

14. The method according to claim 13, wherein the NME protein is NME7.

15. The method as in claim 12, wherein the NME protein is at least 45%, 50%, 55%, 60%, 65%, 70%, 75%, 80%, 85%, 90%, or 95% homologous to the native NME protein sequence of the species of the stem cells to be generated.

16. The method as in claim 14, wherein the NME protein is at least 60% homologous to the native sequence of the species of the stem cells to be generated.

17. The method as in claim 14, wherein the NME protein is at least 70% homologous to the native sequence of the species of the stem cells to be generated.

18.-35. (canceled)

36. A method for generating stem cells, inducing pluripotency in somatic cells or culturing stem cells comprising the steps of contacting cells with an NME protein and/or an anti-MUC1* antibody wherein the NME protein is at least 75% homologous to the sequence of the donor cells and the anti-MUC1* antibody binds to a peptide comprising the sequence of a MUC1* extracellular domain wherein the sequence is at least 75% homologous to the native sequence of the species that donated the cells.

37. A method of treating a person in need of generated tissue or organ, comprising carrying out the steps according to claim 1.

38. A method of generating a first non-human mammal that comprises DNA, molecules, cells, tissue or organ specifically originating from a second mammal that does or does not belong to the same species or genus as the first non-human mammal, comprising introducing cells from the second mammal into the first non-human mammal.

39. The method according to claim 38, wherein the cells from the second mammal are progenitor cells, stem cells or naïve state stem cells.

40. The method according to claim 39, wherein the naïve state stem cells are generated by culturing cells in a media that contains NME.

41. The method according to claim 40, wherein the NME is dimeric NME1, dimeric NME6, NME7-X1 or NME7-AB.

42. The method according to claim 41, wherein the NME has sequence endogenous to the second mammal.

43. The method according to claim 38, wherein the second mammal is human.

44. The method according to claim 38, wherein the first non-human mammal is a rodent, a domesticated mammal, pig, bovine, or a non-human primate.

45. The method according to claim 39, wherein the progenitor cells, stem cells or naïve stem cells are introduced into the fertilized egg, morula, blastocyst, embryo or developing fetus of the first non-human mammal.

46. The method according to claim 38, further comprising:
allowing the first non-human mammal to develop and harvesting from the first non-human mammal molecules, cells, tissues or organs that have incorporated some second mammalian DNA; and
administering to the second mammal in need thereof the molecules, cells, tissues or organs for the treatment or prevention of a disease or condition.

47. The method according to claim 46, wherein the progenitor cells, stem cells or naïve stem cells are iPS cells.

48. The method according to claim 47, wherein somatic cells from which the iPS cells are generated are from the second mammal to which the obtained molecules, cells, tissues or organs for the treatment or prevention of a disease or condition is administered.

49. The method according to claim 46, comprising
determining an organ developmental time period and endogenous genes involved in the development of the organ; and
knocking out or knocking down the endogenous gene during the developmental time period of the organ in the first non-human mammal, wherein the organ is caused to be produced from the cells from the second mammal.

50. The method according to claim 38, wherein the first non-human mammal is close to the second mammal with global sequence identity that is greater than 70%, 75%, 80%, 85%, 90%, or 95% or NME sequence identity that is greater than 45%, 50%, 55%, 60%, 65%, 70%, 75%, 80%, 85%, 90%, or 95%.

51. The method according to claim 46, comprising
determining an organ developmental time period and endogenous genes involved in the development of the organ; and
genetically altering the fertilized egg, cells of morula, cells of the blastocyst, or cells of the embryo or developing fetus of the first non-human mammal such that second mammalian NME7-AB or NME1 is expressed from an inducible or repressable promoter such that the second mammalian cells are timely expanded in response to the non-mammalian NME7-AB or NME1.

52. The method according to claim 39, comprising injecting the second mammalian stem cells into embryo at a later stage of development at the location where the desired organ or tissue would normally develop.

53. The method according to claim 52, further comprising expanding the mammalian stem cells by inducing expression of either first non-human mammalian or second mammalian NME7 or NME1 at that location.

54. The method according to claim 53, comprising expanding the mammalian stem cells by inducing expression of either first non-human mammalian or second mammalian NME1 at that location.

55. The method according to claim 53, wherein a second mammalian promoter is linked to an endogenous first non-human mammalian protein and is expressed at a desired time and location, then introducing an agent that directs the development of the desired tissue.

56. The method according to claim 55, wherein the endogenous first non-human mammalian protein is a protein that induces expression of NME1 or NME7.

57. The method according to claim 56, wherein the endogenous first non-human mammalian protein is a protein that induces expression of NME1.

58. A method of testing for efficacy or toxicity of a potential drug agent in a chimeric animal that expresses some second mammalian DNA or some second mammalian tissue, comprising:
(i) generating a first non-human mammal that comprises DNA, molecules, cells, tissue or organ specifically originating from a second mammal that does or does not belong to the same species or genus as the first non-human mammal, comprising introducing cells from the second mammal into the first non-human mammal; and
(ii) administering a test drug to the first non-human mammal for the effect on the tissue or organ originating from the second mammal.

59. The method according to claim 58, wherein NME is expressed in the first non-human mammal that enhances proliferation of the cells originating from the second mammal.

60. A method discovering a potential drug agent in a chimeric animal that expresses some second mammal DNA or some second mammal tissues, comprising:
(i) generating a first non-human mammal that comprises DNA, molecules, cells, tissue or organ specifically originating from a second mammal that does or does not belong to the same species or genus as the first non-human mammal, comprising introducing cells from the second mammal into the first non-human mammal; and
(ii) administering a compound to the first non-human mammal for the effect on the tissue or organ originating from the second mammal, wherein efficacious effects indicate that the present of a potential drug.

61. The method according to claim 60, wherein NME is expressed in the first non-human mammal that enhances proliferation of the cells originating from the second mammal.
